SEQUENCE LISTING (20 sequences)

SEQ ID NO: 1 - DNA, 10990 bp, synthetic construct
  source 1..10990: mol_type = other DNA
atggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgttggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataagcactcgagtgtagaaataattttgtttaactttaataaggagatataccatggctaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcacgaacgccagcaacttctggaagattttgtattgaccacaaacaaaaatgcgtaaggaaacctttctatgtctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcgtgaacatgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtgttgggccagctaaatcgatgggatgtggcttgctatctttggctccactgtgagcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaggagacacaagcttaatttagtacatgtaggtctagataggaggtgactttaggaggatactttaggagactgttacagatagggacacagagagacactcaaggtaacacccaaagacaaacccaagggtcatccccaacccttagggaaccctcaaggtcaaccgaaggttgactttaggtgattggctgggctttaggtaccccctatgggggaactttaggtccgtacactgtgagatagtcactcagatttttatggtatattttaaaggaccctataggaactccataggtcctctctaagaccctctttaggtgacactgataggatggcctatctttaggtatagaccttaggatagaccattagaggctctttatgggtataccttaggacttgactctatagggacgaagtggtgtgatgcaatcataccgctgaatccctcggtgagccatcagtcggtcaggaagaccctaatcgctacaagtgagtatagagcaaacgacagactccagtgaactgaggtctccagtaagagatgcgtacccaagggcagcacaaagtaccacaagaaatcggtaggtgactctatgccgcagtaggttcaacaataagcggaacattagtctcctccctttatgttggtccttaagttaaggtgatactaatacaatataatcaccattcttagaggtcactctaggtacaccattatgcatactttatgcactctataagtattcattcagtatctatatagtaacctttcgtccctcctctccctatagtgagtcgtattaatttatcgtgtcttttcagtaacttaaagaactgaccatccgtggtctatagtgcatccctatgcagtcctaatgcataaatcaccactcaatgaaagacgtaccgtaaccctcatcgtcctcagagtacacatcaactcctcccacagacatctcaatgatatgcgtagcagcaaccgtaggacgcatcatgtgttcctcaaggaagtcagcaagtacttcaccctcgacctacgcgtcattatcctgtatcacatattctgctgacgcaccggtgcagccttttttctcctgccacatgaagcacttcactgacaccctcatcagtgccaacatagtaaggaattctactacttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacgactagtctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaataccagagctctaagtgaaatcgcgcgtccatccggattgacagctagctcagtcctaggtataatgctagcgtcgacagacgtacaggtcggacgtcgagttccccgcgccagcggggataaaccgctgagcaccgcattagccgctgcattgatgctgagttccccgcgccagcggggataaaccgcaggagatcaacctgccggtcgcgctggcggtgagttccccgcgccagcggggataaaccgctgattgctcacgttggcggcccggctagcgtgagttccccgcgccagcggggataaaccgtgtgccgctgtatgcgcaaacggcggacgtacgagttccccgcgccagcggggataaaccggatttaccaataatcttaaacgggcgaaccaatgagttccccgcgccagcggggataaaccgctgtatcacaataagttacacgtatcggagcggagttccccgcgccagcggggataaaccgttaccgcgctgaggagcgcttcccactgtgcagagttccccgcgccagcggggataaaccgcttacaaaaactgacactgggctctgcactgggagttccccgcgccagcggggataaaccggcgccgagtcattgtgtgccagatctccggaactccgctagcgctgaggtcgactgcggccgctgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttatactagttggtgcccttaatttcagataaaaaaaatccttagctttcgctaaggatgatttcaacgccctcggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgttctgcagtggtgctaggctaggtgtacgtagaagttccccttgacggctagctcagtcctaggtacagtgctagctttctccatacccgtttttttgggctagcaataaggagatatacc

SEQ ID NO: 2 - DNA, 591 bp, Escherichia coli
  source 1..591: mol_type = unassigned DNA
atgatggataacatgcagactgaagcacaaccgacacggacccggatcctcaatgctgccagagagattttttcagaaaatggatttcacagtgcctcgatgaaagccatctgtaaatcttgcgccattagtcccgggacgctctatcaccatttcatctccaaagaagccttgattcaggcgattatcttacaggaccaggagagggcgctggcccgtttccgggaaccgattgaagggattcatttcgttgactatatggtcgagtccattgtctctctcacccatgaagcctttggacaacgggcgctggtggttgaaattatggcggaagggatgcgtaacccacaggtcgccgccatgcttaaaaataagcatatgacgatcacggaatttgttgcccagcggatgcgtgatgcccagcaaaaaggcgagataagcccagacatcaacacggcaatgacttcacgtttactgctggatctgacctacggtgtactggccgatatcgaagcggaagacctggcgcgtgaagcgtcgtttgctcagggattacgcgcgatgattggcggtatcttaaccgcatcctga

SEQ ID NO: 3 - protein, 196 aa, Escherichia coli
MMDNMQTEAQPTRTRILNAAREIFSENGFHSASMKAICKSCAISPGTLYHHFISKEALIQAIILQDQERALARFREPIEGIHFVDYMVESIVSLTHEAFGQRALVVEIMAEGMRNPQVAAMLKNKHMTITEFVAQRMRDAQQKGEISPDINTAMTSRLLLDLTYGVLADIEAEDLAREASFAQGLRAMIGGILTAS

SEQ ID NO: 4 - DNA, 7159 bp, synthetic construct
  source 1..7159: mol_type = other DNA
gccggcgcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatcgcggaacccctatttgtttatttttctaaatacattcaaatatgtatccgctcatgagacaataaccctgataaatgcttcaataatattgaaaaaggaagagtcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaagatgcccagctgtctttcgctgctgagggtgacgatcccgcggatcccgggcccgtcgactgcagaggcctgcatgcaagcttggcgtaatcggcgaacgtggtccggataatacgactcactatagggcgaattgggcccgacgtcgcatgctcccggccgccatggccgcgggatataaggtcgacaagaaggagatatacatatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaagtcgacactagttaatacgactcactatagggagattgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaactcgaggatatcgctagc

SEQ ID NO: 5 - DNA, 7172 bp, synthetic construct
  source 1..7172: mol_type = other DNA
gccggcgcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaagatgcccagctgtctttcgctgctgagggtgacgatcccgcggatcccgggcccgtcgactgcagaggcctgcatgcaagcttggcgtaatcggcgaacgtggtccggtaatacgactcactatagggcgaattgggcccgacgtcgcatgctcccggccgccatggccgcgggattaaggtcgacaagaaggagatatacatatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcagttaaagacaatgaaccagaatagctggcaagcccgcaatgaagcactgcagttccgcaacgaagccgagacatttcgcaaccagaccgaagtgtttaaaaacgagagcggcaccaacgccaccaatacaaaacagtggcgcgatgaagccaacggcagtcgtgatgaagccgaacaattcaagaacaccgccggccagtatgccaccagtgccggtaatagcgcaaccgcagcccatcagagcgaagttaacgccgagaatagcgccaccgcagccgccaatagtgcaaatctggcagagcagcatgccgatcgtgccgagcgcgaagccgataaactgggcaactttaacggtctggccggcgcaattgatcgcgtggatggcaccaacgtgtactggaaaggtggcatccatgcaaatggtcgtttatatctgaccagcgatggctttgattgcggccagtaccagcagttctttggtggcagtgccggtcgttatagcgttatggaatggggcgatgaaaacggctggttaatgcatgtgcagcgccgtgaatggaccacagccattggcgacaacatccagctggtggtgaatggccacatcattgcccaaggtggcgatatgaccggtcctctgaaactgcagaacggccacgctttatatttagaaagcgccagcgataaggcccagtatatcttaagcaaagatggtaatcgcaataactggtatatcggtcgcggcagcgataacaacaatgattgcaccttccacagttacgtgtacggcaccaatttaacactgaagccggattatgccgtggtgaacaaacgctttcatgtgggtcaagctgtggtggccacagatggcaacatccaaggtaccaaatggggcggtaagtggctggacgcctatctgaacgatacctatgttaagaaaactatggcttggacacaagtttgggcagcagcaagcggtagctatatgggtggtggtagccagaccgacactttaccgcaagatttacgctttcgcaatatctggattaaaacccgtaataactactggaatttctttcgcactggtccggatggcatttactatttaagcgcagagggcggttggctgaaattccagatccacagcaacggccgtgtgttcaagaatattgccgaccgtgacgcaccgccgacagcaattgcagttgaagatgtttaagtcgacactagttaatacgactcactatagggagattgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaactcgaggatatcgctagc

SEQ ID NO: 6 - DNA, 4325 bp, synthetic construct
  source 1..4325: mol_type = other DNA
aacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcgaggatccagtgccaacatagtaagccagtagagctcgccggcaagctgagcaccgcattagccgctgcattgatgctgggccgagcgattaagcgtgacaaagtgtgccgctgtatgcgcaaacggcggacgtacagggccgcgcagagttagttcataagaaccattcgctaaactcgaacaggactttggcggggactttaggtcgagcacagtaaggtctatgatacgaccatgctccaaaatgccctaggccatggtaagtacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgatagtacatgtaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggcgggatcgtcaccctcagcagcgaaagacagctgtcggtcagagcgtcattgcgaagctgagtgtgatcgatgccatcagcgaagggcccaaactccgagcgattaagcgtttgctggctgtcacgcctgcctgttgcttgcttggacttgcgatgtacgtgctcagctgtctttcgctgctgagggtgacgatcccgcgagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgacatgtactacgatcaaaggatcttcttgagatcctttttttctgcgcgtaatcttttgccctgtaaacgaaaaaaccacctggggaggtggtttgatcgaaggttaagtcagttggggaactgcttaaccgtggtaactggctttcgcagagcacagcaaccaaatctgtccttccagtgtagccggactttggcgcacacttcaagagcaaccgcgtgtttagctaaacaaatcctctgcgaactcccagttaccaatggctgctgccagtggcgttttaccgtgcttttccgggttggactcaagtgaacagttaccggataaggcgcagcagtcgggctgaacggggagttcttgcttacagcccagcttggagcgaacgacctacaccgagccgagataccagtgtgtgagctatgagaaagcgccacacttcccgtaagggagaaaggcggaacaggtatccggtaaacggcagggtcggaacaggagagcgcaagagggagcgacccgccggaaacggtggggatctttaagtcctgtcgggtttcgcccgtactgtcagattcatggttgagcctcacggctcccacagatgcaccggaaaagcgtctgtttatgtgaactctggcaggagggcggagcctatggaaaaacgccaccggcgcggccctgctgttttgcctcacatgttagtcccctgcttatccacggaatctgtgggtaactttgtatgtgtccgcagcgcaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtattgacagctagctcagtcctagggactatgctagctttctccatacccgtttttttgggctagcgaattcgagctcggtacccggggatcctctagagaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcacgtcgacctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgactgcaggcatgcaagcttggctgttttggcggatgagagaagattttcagcctgatacagattaaatcag

SEQ ID NO: 7 - DNA, 35422 bp, synthetic construct
  source 1..35422: mol_type = other DNA
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgaaaaacaggtattgacaacatgaagtaacatgcagtaagatacaaatcgctaggtaacactagcagcgtcaaccgggcgcacagtgccttctaggtgacttaagcgcaccacggcacataaggtgaaacaaaacggttgacaacatgaagtaaacacggtacgatgtaccacatgaaacgacagtgagtcaccacactgaaaggtgatgcggtctaacgaaacctgacctaagacgctctttaacaatctggtaaatagctcttgagtgcatgactagcggataactcaagggtatcgcaaggtgccctttatgatattcactaataactgcacgaggtaacacaagatggctatgtctaacatgacttacaacaacgttttcgaccacgcttacgaaatgctgaaagaaaacatccgttatgatgacatccgtgacactgatgacctgcacgatgctattcacatggctgccgataatgcagttccgcactactacgctgacatctttagcgtaatggcaagtgagggcattgaccttgagttcgaagactctggtctgatgcctgacaccaaggacgtaatccgcatcctgcaagcgcgtatctatgagcaattaacgattgacctctgggaagacgcagaagacttgctcaatgaatacttggaggaagtcgaggagtacgaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcactttacttatgagggagtaatgtatatgcttactatcggtctactcaccgctctaggtctagctgtaggtgcatcctttgggaaggctttaggtgtagctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgatggcttaaagaaaggttatatgcccaatggcacactatacgctgcaaatcggcgaatagtgagaacttggcgagagaacaacctcgaacgccgcaaggacaagagagggcggcgtggcatagacgaaaggaaaaggttaaagccaagaaactcgccgcacttgaacaggcactagccaacacactgaacgctatctcataacgaacataaaggacacaatgcaatgaacattaccgacatcatgaacgctatcgacgcaatcaaagcactgccaatctgtgaacttgacaagcgtcaaggtatgcttatcgacttactggtcgagatggtcaacagcgagacgtgtgatggcgagctaaccgaactaaatcaggcacttgagcatcaagattggtggactaccttgaagtgtctcacggctgacgcagggttcaagatgctcggtaatggtcacttctcggctgcttatagtcacccgctgctacctaacagagtgattaaggtgggctttaagaaagaggattcaggcgcagcctataccgcattctgccgcatgtatcagggtcgtcctggtatccctaacgtctacgatgtacagcgccacgctggatgctatacggtggtacttgacgcacttaaggattgcgagcgtttcaacaatgatgcccattataaatacgctgagattgcaagcgacatcattgattgcaattcggatgagcatgatgagttaactggatgggatggtgagtttgttgaaacttgtaaactaatccgcaagttctttgagggcatcgcctcattcgacatgcatagcgggaacatcatgttctcaaatggagacgtaccatacatcaccgacccggtatcattctcgcagaagaaagacggtggcgcattcagcatcgaccctgaggaactcatcaaggaagtcgaggaagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatgatccgtcagcctgcagttcgaagttcctatactttctagagaataggaacttcgaagcagctccagcctacattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaagatccgtcagcctgcagttcgaagttcctatactttctagagaataggaacttcgaagcagctccagcctacattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 8 - DNA, 591 bp, synthetic construct
  source 1..591: mol_type = other DNA
atgatggataacatgcagactgaagcacaaccgacacggacccggatcctcaatgctgccagagagattttttcagaaaatggatttcacagtgcctcgatgaaagccatctgtaaatcttgcgccattagtcccgggacgctctatcaccatttcatctccaaagaagccttgattcaggcgattatcttacaggaccaggagagggcgctggcccgtttccgggaaccgattgaagggattcatttcgttgactatatggtcgagtccattgtctctctcacccatgaagcctttggacaacgggcgctggtggttgaaattatggcggaagggatgcgtaacccacaggtcgccgccatgcttaaaaatgcgcatatgacgatcacggaatttgttgcccagcggatgcgtgatgcccagcaaaaaggcgagataagcccagacatcaacacggcaatgacttcacgtttactgctggatctgacctacggtgtactggccgatatcgaagcggaagacctggcgcgtgaagcgtcgtttgctcagggattacgcgcgatgattggcggtatcttaaccgcatcctga

SEQ ID NO: 9 - protein, 196 aa, synthetic construct
MMDNMQTEAQPTRTRILNAAREIFSENGFHSASMKAICKSCAISPGTLYHHFISKEALIQAIILQDQERALARFREPIEGIHFVDYMVESIVSLTHEAFGQRALVVEIMAEGMRNPQVAAMLKNAHMTITEFVAQRMRDAQQKGEISPDINTAMTSRLLLDLTYGVLADIEAEDLAREASFAQGLRAMIGGILTAS

SEQ ID NO: 10 - DNA, 591 bp, synthetic construct
  source 1..591: mol_type = other DNA
atgatggataacatgcagactgaagcacaaccgacacggacccggatcctcaatgctgccagagagattttttcagaaaatggatttcacagtgcctcgatgaaagccatctgtaaatcttgcgccattagtcccgggacgctctatcaccatttcatctccaaagaagccttgattcaggcgattatcttacaggaccaggagagggcgctggcccgtttccgggaaccgattgaagggattcatttcgttgactatatggtcgagtccattgtctctctcacccatgaagcctttggacaacgggcgctggtggttgaaattatggcggaagggatgcgtaacccacaggtcgccgccatgcttaaaaataagcatatgacgatcacggaatttgttgcccagcggatgcgtgatgcccagcaaaaaggcgagataagcccagacatcaacacggcaatgacttcacgtttactgctggatctgacctttggtgtactggccgatatcgaagcggaagacctggcgcgtgaagcgtcgtttgctcagggattacgcgcgatgattggcggtatcttaaccgcatcctga

SEQ ID NO: 11 - protein, 196 aa, synthetic construct
MMDNMQTEAQPTRTRILNAAREIFSENGFHSASMKAICKSCAISPGTLYHHFISKEALIQAIILQDQERALARFREPIEGIHFVDYMVESIVSLTHEAFGQRALVVEIMAEGMRNPQVAAMLKNKHMTITEFVAQRMRDAQQKGEISPDINTAMTSRLLLDLTFGVLADIEAEDLAREASFAQGLRAMIGGILTAS

SEQ ID NO: 12 - DNA, 591 bp, synthetic construct
  source 1..591: mol_type = other DNA
atgatggataacatgcagactgaagcacaaccgacacggacccggatcctcaatgctgccagagagattttttcagaaaatggatttcacagtgcctcgatgaaagccatctgtaaatcttgcgccattagtcccgggacgctctatcaccatttcatctccaaagaagccttgattcaggcgattatcttacaggaccaggagagggcgctggccgccttccgggaaccgattgaagggattcatttcgttgactatatggtcgagtccattgtctctctcacccatgaagcctttggacaacgggcgctggtggttgaaattatggcggaagggatgcgtaacccacaggtcgccgccatgcttaaaaataagcatatgacgatcacggaatttgttgcccagcggatgcgtgatgcccagcaaaaaggcgagataagcccagacatcaacacggcaatgacttcacgtttactgctggatctgacctacggtgtactggccgatatcgaagcggaagacctggcgcgtgaagcgtcgtttgctcagggattacgcgcgatgattggcggtatcttaaccgcatcctga

SEQ ID NO: 13 - protein, 196 aa, synthetic construct
MMDNMQTEAQPTRTRILNAAREIFSENGFHSASMKAICKSCAISPGTLYHHFISKEALIQAIILQDQERALAAFREPIEGIHFVDYMVESIVSLTHEAFGQRALVVEIMAEGMRNPQVAAMLKNKHMTITEFVAQRMRDAQQKGEISPDINTAMTSRLLLDLTYGVLADIEAEDLAREASFAQGLRAMIGGILTAS

SEQ ID NO: 14 - DNA, 1812 bp, Escherichia coli
  source 1..1812: mol_type = unassigned DNA
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 15 - protein, 603 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 16 - DNA, 1374 bp, Escherichia coli
  source 1..1374: mol_type = unassigned DNA
atgaatcaacaactctcctggcgcaccatcgtcggctacagcctcggtgacgtcgccaataacttcgccttcgcaatgggggcgctcttcctgttgagttactacaccgacgtcgctggcgtcggtgccgctgcggcgggcaccatgctgttactggtgcgggtattcgatgccttcgccgacgtctttgccggacgagtggtggacagtgtgaatacccgctggggaaaattccgcccgtttttactcttcggtactgcgccgttaatgatcttcagcgtgctggtattctgggtgctgaccgactggagccatggtagcaaagtggtgtatgcatatttgacctacatgggcctcgggctttgctacagcctggtgaatattccttatggttcacttgctaccgcgatgacccaacaaccacaatcccgcgcccgtctgggcgcggctcgtgggattgccgcttcattgacctttgtctgcctggcatttctgataggaccgagcattaagaactccagcccggaagagatggtgtcggtataccatttctggacaattgtgctggcgattgccggaatggtgctttacttcatctgcttcaaatcgacgcgtgagaatgtggtacgtatcgttgcgcagccgtcattgaatatcagtctgcaaaccctgaaacggaatcgcccgctgtttatgttgtgcatcggtgcgctgtgtgtgctgatttcgacctttgcggtcagcgcctcgtcgttgttctacgtgcgctatgtgttaaatgataccgggctgttcactgtgctggtactggtgcaaaacctggttggtactgtggcatcggcaccgctggtgccggggatggtcgcgaggatcggtaaaaagaataccttcctgattggcgctttgctgggaacctgcggttatctgctgttcttctgggtttccgtctggtcactgccggtggcgttggttgcgttggccatcgcttcaattggtcagggcgttaccatgaccgtgatgtgggcgctggaagctgataccgtagaatacggtgaatacctgaccggcgtgcgaattgaagggctcacctattcactattctcatttacccgtaaatgcggtcaggcaatcggaggttcaattcctgcctttattttggggttaagcggatatatcgccaatcaggtgcaaacgccggaagttattatgggcatccgcacatcaattgccttagtaccttgcggatttatgctactggcattcgttattatctggttttatccgctcacggataaaaaattcaaagaaatcgtggttgaaattgataatcgtaaaaaagtgcagcagcaattaatcagcgatatcactaattaa

SEQ ID NO: 17 - protein, 457 aa, Escherichia coli
MNQQLSWRTIVGYSLGDVANNFAFAMGALFLLSYYTDVAGVGAAAAGTMLLLVRVFDAFADVFAGRVVDSVNTRWGKFRPFLLFGTAPLMIFSVLVFWVLTDWSHGSKVVYAYLTYMGLGLCYSLVNIPYGSLATAMTQQPQSRARLGAARGIAASLTFVCLAFLIGPSIKNSSPEEMVSVYHFWTIVLAIAGMVLYFICFKSTRENVVRIVAQPSLNISLQTLKRNRPLFMLCIGALCVLISTFAVSASSLFYVRYVLNDTGLFTVLVLVQNLVGTVASAPLVPGMVARIGKKNTFLIGALLGTCGYLLFFWVSVWSLPVALVALAIASIGQGVTMTVMWALEADTVEYGEYLTGVRIEGLTYSLFSFTRKCGQAIGGSIPAFILGLSGYIANQVQTPEVIMGIRTSIALVPCGFMLLAFVIIWFYPLTDKKFKEIVVEIDNRKKVQQQLISDITN

SEQ ID NO: 18 - DNA, 1266 bp, Escherichia coli
  source 1..1266: mol_type = unassigned DNA
atgagaaaaatagtggccatggccgttatttgcctgacggctgcctctggccttacctctgcttatgcggcgcaactggctgacgatgaagcgggactacgcatcagactgaaaaacgaattgcgcagggcggataagcccagtgctggcgcgggaagagatatttacgcatgggtacagggaggattgctcgatttcaatagtggttattattccaatattattggcgttgaaggcggggcgtattatgtttataaattaggtgctcgtgctgatatgagtacccggtggtatcttgatggtgataaaagttttggctttgccctgggggcagtaaaaataaaacccagtgaaaatagcctgcttaaattaggtcgcttcgggacggattatagttatggtagcttaccttatcgtattccgttaatggctggcagttcgcaacgtacattaccgacagtttctgaaggagcattaggttattgggctttaacaccaaatattgatctgtggggaatgtggcgttcacgagtatttttatggactgattcaacaaccggtattcgtgatgaaggggtgtataacagccagacgggaaaatacgataaacatcgcgcacgttcttttttagccgccagttggcatgatgataccagtcgctattctctgggggcatcggtacagaaagatgtttccaatcagatacaaagtattctcgagaaaagcataccgctcgacccgaattatacgttgaaaggggagttgctcggcttttacgcgcagctcgaaggtttaagtcgtaataccagccagcccaatgaaacggcgttggttagtggacaattgacctggaatgcgccgtggggaagtgtatttggcagtggtggttatttgcgccatgcaatgaatggtgccgtggtggataccgacattggctatcccttttcattaagtcttgatcgtaaccgtgaaggaatgcagtcctggcaattgggcgtcaactatcgtttaacgccgcaatttacgctgacatttgcaccgattgtgactcgcggctatgaatccagtaaacgagatgtgcggattgaaggcacgggtatcttaggtggtatgaactatcgggtcagcgaagggccgttacaagggatgaatttctttcttgctgccgataaagggcgggaaaagcgcgatggcagtacgctgggcgatcgcctgaattactgggatgtgaaaatgagtattcagtatgactttatgctgaagtaa

SEQ ID NO: 19 - protein, 421 aa, Escherichia coli
MRKIVAMAVICLTAASGLTSAYAAQLADDEAGLRIRLKNELRRADKPSAGAGRDIYAWVQGGLLDFNSGYYSNIIGVEGGAYYVYKLGARADMSTRWYLDGDKSFGFALGAVKIKPSENSLLKLGRFGTDYSYGSLPYRIPLMAGSSQRTLPTVSEGALGYWALTPNIDLWGMWRSRVFLWTDSTTGIRDEGVYNSQTGKYDKHRARSFLAASWHDDTSRYSLGASVQKDVSNQIQSILEKSIPLDPNYTLKGELLGFYAQLEGLSRNTSQPNETALVSGQLTWNAPWGSVFGSGGYLRHAMNGAVVDTDIGYPFSLSLDRNREGMQSWQLGVNYRLTPQFTLTFAPIVTRGYESSKRDVRIEGTGILGGMNYRVSEGPLQGMNFFLAADKGREKRDGSTLGDRLNYWDVKMSIQYDFMLK

SEQ ID NO: 20 - DNA, 389 bp, Escherichia coli
  source 1..389: mol_type = unassigned DNA
ttctctctctttttcggcgggctggtgataactgtgcccgcgtttcatatcgtaatttctctgtgcaaaaattatccttcccggcttcggagaattccccccaaaatattcactgtagccatatgtcatgagagtttatcgttcccaatacgctcgaacgaacgttcggttgcttattttatggcttctgtcaacgctgttttaaagattaatgcgatctatatcacgctgtgggtattgcagtttttggttttttgatcgcggtgtcagttctttttatttccatttctcttccatgggtttctcacagataactgtgtgcaacacagaattggttaactaatcagattaaaggttgaccagtattattatcttaatgaggagtccct

(19) United States
(12) Patent Application Publication
(10) Pub. No.: US 2025/0084398 A1
(43) Pub. Date: Mar. 13, 2025

(54) SYSTEMS FOR MODULATING DRUG METABOLISM BY CELLS, METHODS AND COMPOSITIONS THEREOF

(71) Applicant: TROBIX BIO LTD, Netanya (IL)

(72) Inventors: Adi ELKELES, Tel Mond (IL); Ziv LIFSHITZ, Kfar Yona (IL); Lirit DUCHOVANI, Ramat Gan (IL)

(21) Appl. No.: 18/294,978

(22) PCT Filed: Aug. 1, 2022
(86) PCT No.: PCT/IL2022/050826

§ 371 Date: Feb. 2, 2024

Related U.S. Application Data
(60) Provisional application No. 63/203,853, filed on Aug. 2, 2021.

(51) Int. Cl. C12N 15/10; A61K 31/519; A61K 38/00; C12N 7/00; C12N 9/22; C12N 9/24; C12N 15/11

(52) U.S. Cl. CPC C12N 15/102; A61K 31/519; C12N 7/00; C12N 9/22; C12N 9/2402; C12N 15/111; A61K 38/00; C12N 2310/20; C12N 2320/31; C12N 2795/10042; C12Y 302/01031

(57) ABSTRACT

The present disclosure provides systems for modulating drug metabolism, specifically in the gut microbiome, by modifying target cell/s and/or population of cells comprising the target cell/s. The disclosed systems comprise the following components: (a) at least one drug metabolism modulating component comprising at least one nucleic acid sequence encoding and/or modulating at least one element participating in metabolism of at least one drug, at least one cas gene and at least one Clustered. Regularly Interspaced Short Palindromic Repeat (CRISPR) array; and (b) at least one selective component comprising at least one protospacer. The at least one protospacer is targeted by at least one spacer of the CRISPR array of (a), thereby inactivating said selective component. The present disclosure further provides, compositions and methods using the disclosed systems and any components thereof in modulation of drug metabolism and in therapeutic applications.

### FIELD OF THE INVENTION

The present disclosure relates to the field of manipulation of the microbiome. More specifically, the present disclosure relates to modulation of drug metabolism by specific and targeted modulation of elements that participate in drug metabolism in microbiome cells, specifically, cells of the gut microbiome, e.g., bacterial cells.

### BACKGROUND ART

References considered to be relevant as background to the presently disclosed subject matter are listed below:
WO 2016/084088
WO 2018/002940
Ahmad, S., Hughes, M. A., Lane, K. T., Redinbo, M. R., Yeh, L.-A., & Scott, a. J. (2011). A High Throughput Assay for Discovery of Bacterial β-Glucuronidase Inhibitors. Curr Chem Genomics.
Boelsterli, U. A., Redinbo, M. R., & Saitta, K. S. (2013). Multiple NSAID-induced hits injure the small intestine: underlying mechanisms and novel strategies. Toxicol Sci.
Coyle, V. M., Lungulescu, D., Toganel, C., Niculescu, A., Pop, S., Ciuleanu, T., Wilson, R. H. (2013). A randomised double-blind placebo-controlled phase II study of AGI004 for control of chemotherapy-induced diarrhoea. British Journal of Cancer volume.
Fittkau, M., Voigt, W., Holzhausen, H.-J., & Schmoll, H. J. (2004). Saccharic acid 1.4-lactone protects against CPT-11-induced mucosa damage in rats. J Cancer Res Clin Oncol.
Higuchi, K., Yoda, Y., Amagase, K., Kato, S., Tokioka, S., Murano, M., Umegaki, E. (2009). Prevention of NSAID-Induced Small Intestinal Mucosal Injury: Prophylactic Potential of Lansoprazole. J Clin Biochem Nutr.
Kodawara, T., Masuda, S., Yano, Y., Matsubara, K., Nakamura, T., & Masada, M. (2014). Inhibitory effect of ciprofloxacin on β-glucuronidase-mediated deconjugation of mycophenolic acid glucuronide. Biopharm Drug Dispos.
Koppel, N., Rekdal, V. M., & Balskus, E. P. (2017). Chemical transformation of xenobiotics by the human gut microbiota. Science.
Peekhaus, N., & Conway, T. (1998). What's for dinner?: Entner-Doudoroff metabolism in Escherichia coli. J Bacteriol.
Pellock, S. J., Creekmore, B. C., Walton, W. G., Mehta, N., Biernat, K. A., Cesmat, A. P. Redinbo, M. R. (2018). Gut Microbial β-Glucuronidase Inhibition via Catalytic Cycle Interception. ACS Cent Sci.
Sakamoto, H., Yokota, H., Kibe, R., Sayama, Y., & Yuasa, A. (2002). Excretion of bisphenol A-glucuronide into the small intestine and deconjugation in the cecum of the rat. Biochim. Biophys. Acta.
Tobin, P. J., Dodds, H. M., Clarke, S., Schnitzler, M., & Rivory, L. P. (2003). The relative contributions of carboxylesterase and beta-glucuronidase in the formation of SN-38 in human colorectal tumours. Oncol Rep.
Wallace, B. D., Roberts, A. B., Pollet, R. M., Ingle, J. D., Biernat, K. A., Pellock, S. J., Redinbo, M. R. (2016). Structure and Inhibition of Microbiome β-Glucuronidases Essential to the Alleviation of Cancer Drug Toxicity. Chem Biol.
Weisburger, J. H., Barnes, W. S., Lovelette, C. A., Tong, C., Tanaka, T., & Williams, G. M. (1986). Genotoxicity, carcinogenicity, and mode of action of the fried food mutagen 2-amino-3-methylimidazo[4,5-f]quinoline (IQ). Environmental Health Perspectives.

Acknowledgement of the above references herein is not to be inferred as meaning that these are in any way relevant to the patentability of the presently disclosed subject matter.

### BACKGROUND OF THE INVENTION

The gut microbiome, a highly complex and dynamic ecosystem, comprises up to 1 Kg of bacteria in adult humans. The gut microbiome within an individual is established early in life and remains relatively stable during life-time, but its composition and/or function may be influenced by a range of factors such as diet, probiotics, and drugs, especially antibiotics.

This aggregate of trillions of microorganisms is now known to play a critical role in human health and predisposition to disease. It also performs various essential biological functions such as synthesis of vitamins, development and modulation of the immune system, bacterial defense, the intestinal response to epithelial cell injury and nutrient metabolism. In addition, the gut microbiota can produce a number of neurotransmitters, including serotonin, dopamine and noradrenaline.

Researchers are now referring to the gut microbiota as the “second brain” or “second genome” due to its profound physiological role.

As a by-product of their symbiotic relationship with the host, the gut microbiome directly and indirectly affects the pharmacological and/or toxicological characteristics of numerous drugs, ability that was first recognized over 40 years back. To date, more than 30 drugs have been identified as substrates for intestinal bacteria and these numbers are continuously and swiftly rising. Understanding the role of the gut microbiome in drug response may enable the development of microbiome-targeting approaches that enhance drug efficacy. Thus, a research field, termed pharmacomicrobiomics, has been emerged to explore the gut microbiome impact on drugs absorption, distribution, metabolism and excretion (ADME).

The ability of the gut microbiome to modify drugs and compounds is owed to its capability of preforming a wide range of metabolic reactions. The most important reactions involve reductive metabolism and hydrolytic reactions (particularly on conjugates). Demethylation, deamination, dehydroxylation, deacylation, decarboxylation and oxidation have also been described and are no less important than reductive metabolism.

Direct microbial effects on drug response are the chemical transformations of drug compounds by gut microbiome that influence a drug's bioavailability or bioactivity and its toxicity (Koppel, Rekdal, & Balskus, 2017). Toxicity occurs when the bacterial transformation of a drug leads to the generation of metabolites that have harmful effects on the host. The human body uses glucuronidation, which is one of the most important pathway in the phase II metabolism of xeno- and endobiotics, to detoxify hundreds of drugs and endogenous compounds, including bilirubin, bile acids and thyroxine. This is done by increasing their solubility in water, and, in this way, allows for their subsequent elimination from the body through urine or the GI tract (via bile from the liver) at a significantly increased rate. In glucuronidation, a glucuronic acid (GlcA) is conjugated to a substrate via glycosidic bonds. This process is mediated by uridine 5′-diphosphate glucuronyltransferases (UGTs) enzymes, group of enzymes that have been found in all major body organs, although glucuronidation take place mainly in the liver. The resulting end products of this process are named β-D-glucuronides (or glucuronides).

Many of gut microbiome members can metabolite glucuronides, which are abundant in the gut, to liberate GlcA from conjugated compounds to be used as an energy source. The GlcA enter the Entner-Doudoroff pathway, a bacterial alternative to glycolysis that catabolizes sugar acids and shunts the resulting pyruvate into the TCA cycle (Peekhaus & Conway, 1998).

The bacterial-mediated removal of the GlcA from glucuronides in the gut reactivates and effectively reverse the actions of the UGT-mediated mammalian inactivation of these compounds. The hydrolysis of the glucuronides glycosidic bonds is catalyzed by a group of bacterial enzymes termed b-glucuronidases, or GUS. Perhaps the most extensively studied example of gut microbiome-mediated drug toxicity is the case of CPT-11 (Irinotecan). Irinotecan is one of the most commonly used chemotherapeutic agents for colon cancer. CPT-11 is a prodrug that is activated in the liver to its toxic form, SN38, an antineoplastic topoisomerase I poison. Liver SN-38 is inactivated to SN38G by UGT-mediated glucuronidation. Once in the intestines, SN38G serves as a substrate for GUS enzymes that reactivate SN38 in situ (Tobin, Dodds, Clarke, Schnitzler, & Rivory, 2003). The free intestinal SN38, which represents the dose-limiting toxicity of irinotecan, is responsible for Irinotecan-induced diarrhea, which was reported overall incidence from 40% to 80% (Coyle, et al., 2013).

Several attempts to overcome Irinotecan-induced diarrhea have been made; oral alkalinization, addition of phenobarbital and cyclosporine, activated charcoal etc. The feasibility of using antibiotics prior to CPT-11 was explored showing promising clinical data, in which combining cholestyramine/levofloxacin lowered Irinotecan-related delayed diarrhea severity. Moreover, (Kodawara, et al., 2014) demonstrated the inhibitory effect of ciprofloxacin on the activity of β-glucuronidase. Nevertheless, this approach has several drawbacks: dysbiosis of the gut microbiota is not recommended for immunocompromised patients and elimination of symbiotic gut members of the microbiome increases the chances of infections by pathogenic.

The gut microbiome GUS activity was shown to be the cause of elevated concentrations of intestinal toxic metabolites, and specifically SN38, and thus the cause of Irinotecan-induced diarrhea. Therefore, a potent and specific GUS inhibitor, which will not hamper the treatment efficacy of Irinotecan nor disrupt the native microbiota, would be of great clinical value and several approaches to limit or abolish this microbial activity were investigated.

Fittkau et al., (Fittkau, Voigt, Holzhausen, & Schmoll, 2004) and then Wallace, et al., (Wallace, et al., 2016) screened a library of compounds and investigated their effect on GUS activity. They identified several potent inhibitors that reduced the GI toxicity associated with irinotecan in rats. They also showed that these inhibitors were selective for the bacterial GUS by more than 1,000-fold over the human enzyme ortholog. This is important since the E. coli GUS shares 50% amino acid sequence identity with highly conserved active sites to the human GUS, which is an important lysosomal enzyme for the degradation of glycosaminoglycan.

Pellock, et al., (Pellock, et al., 2018) discovered a group of piperazine-containing GUS inhibitors that are selective for microbial GUS enzymes and inhibit GUS by forming covalent inhibitor-GlcA complexes in the GUS active site.

Ahmad, et al., (Ahmad, et al., 2011) screened a library of FDA-approved drugs to explore whether any of these known drugs have GUS inhibitory activity. This screen identified nialamide, isocarboxazid, and amoxapine to have potential to be repurposed as therapeutics to reduce diarrhea associated with irinotecan chemotherapy.

Irinotecan was not the only case of GUS-mediated reactivation of compounds results in toxicity. Via the same mechanism as in Irinotecan, bacterial GUS can also induce toxicity of non-steroidal anti-inflammatory drugs (NSAIDs), which can cause gastroduodenal mucosal lesions in up to 50% of users (Higuchi, et al., 2009). When glucuronidated NSAIDs secreted via the hepatobiliary pathway and reach the distal small intestinal lumen, bacterial GUS metabolize these compounds increase concentrations of non-glucuronidated NSAIDs in the lumen. These molecules are further metabolized by intestinal cytochrome P450 to potentially reactive intermediates that induce severe endoplasmic reticulum stress or mitochondrial stress leading to cell death (Boelsterli, Redinbo, & Saitta, 2013).

In addition, Weisburger, et al., (Weisburger, et al., 1986) showed that the carcinogenicity of hetero-aromatic compounds that is produced during meat processing was found to be associated with gut bacterial GUS. Sakamot ET AL., (Sakamoto, Yokota, Kibe, Sayama, & Yuasa, 2002) demonstrated that bacterial GUS activity in the cecum delays elimination of the endocrine-disrupting agent, bisphenol A. Thus, bacterial GUS enzymes, which demonstrated a wide range of activity against many compounds, appear to play an important role in health and disease by metabolizing glucuronides in the gut. It can significantly affect the pharmacokinetics of many exogenous and endogenous compounds by directly regulating local and systemic levels of these compounds.

WO 2016/084088 relates to kits, systems and methods for interfering with horizontal transfer of a pathogenic gene between bacteria and for preventing a pathologic condition in a mammalian subject caused by a bacterial infection.

WO 2018/002940 relates to a platform for the preparation of improved nucleic acid delivery vehicles, specifically, vehicles having an extended host recognition ability, compositions and uses thereof.

There is therefore a need for efficient manipulation of the microbiota in order to limit dose toxicity of drugs thereby improving drug metabolism and efficacy. This need is addressed by the present disclosure.

### SUMMARY OF THE INVENTION

A first aspect of the present disclosure relates to a system for modulating drug metabolism by modifying target cell/s and/or target cell population. The cell or cell population modified by the system of the present disclosure exhibits modified metabolism of at least one drug. More specifically, the system of the present disclosure comprises at least two elements.

One component or part (a), comprises at least one drug metabolism modulating component. This component comprises at least one nucleic acid sequence encoding and/or modulating at least one element participating in metabolism of at least one drug, at least one cas gene and at least one Clustered, Regularly Interspaced Short Palindromic Repeat (CRISPR) array. The second component or part of the system disclosed herein (b), comprises at least one selective component comprising at least one protospacer, wherein said at least one protospacer is targeted by at least one spacer of the CRISPR array of (a), thereby inactivating said selective component. In some embodiments, the target cells are bacterial cells.

A further aspect of the present disclosure relates to a method for modulating drug metabolism of a target cell/s and/or cell population (e.g., bacterial cells). In some embodiments, the method comprising at least one of the following steps:

One step (a), that in some embodiment may be a first step, that involves contacting the cell/s or any cell population comprising the cell/s with at least one drug metabolism modulating component comprising at least one nucleic acid sequence encoding or modulating at least one element participating in metabolism of at least one drug, at least one cas gene and at least one CRISPR array. In another step (b), that may be in some embodiments a second step that involves contacting the cells or any cell population comprising the cell/s with at least one selective component comprising at least one protospacer. It should be understood that the at least one protospacer is targeted by at least one spacer of the CRISPR array of (a) to inactivate the selective component. Exposing and/or contacting the target cell and/or cell population to the drug metabolism modulating component and/or to the selective component, and/or to any system or composition comprising at least one of (a) and (b) components, leads to modulation of the drug metabolism of the target bacterial cell, and moreover, selecting and/or enriching a population of cells exhibiting modified metabolism of at least one drug.

A further aspect of the present disclosure relates to at least one cell and/or a population of cells, or any composition or product thereof, exhibiting modified metabolism of at least one drug. In some specific embodiments, the cells are bacterial cells and the disclosure thus provides bacterial cells and populations thereof exhibiting modified metabolism of at least one drug In some embodiments, the cell and/or cell population is prepared by a method comprising at least one of the following steps. In one step (a), that may be in some embodiments a first step, the method involves contacting the cell/s or any cell population comprising the target cell/s with at least one drug metabolism modulating component comprising at least one nucleic acid sequence encoding or modulating at least one element participating in metabolism of at least one drug, at least one cas gene and at least one CRISPR array. In another step (b), that may be in some embodiments the next step or the second step (b), contacting the cells or any cell population comprising the cell/s with at least one selective component comprising at least one protospacer. It should be understood that at least one protospacer is targeted by at least one spacer of the CRISPR array of (a), to inactivate said selective component.

A further aspect of the present disclosure relates to a combined composition, specifically, a pharmaceutical composition comprising at least one drug and at least one of the systems of the invention that lead to modulated metabolism of the drug of the disclosed composition, or any cell or cell population that display modulated metabolism of the drug. In more specific embodiments, the composition disclosed herein may comprise:
(I) at least one drug metabolism modulatory system comprising at least one of: (a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and (b) at least one selective component comprising at least one protospacer. It should be noted that wherein said at least one protospacer is targeted by at least one spacer of the CRISPR array of (a), to inactivate said selective component. In some alternative or additional embodiments, the composition may further comprise, or alternatively comprise in addition to the drug, also (II), at least one bacterial cell and/or a population of the bacterial cells, or any composition or product thereof, exhibiting modified metabolism of the at least one drug. Still further, in some embodiments the composition optionally further comprises at least one of pharmaceutically acceptable carrier/s, diluent/s, excipient/s and additive/s.

A further aspect of the present disclosure relates to a method for modulating the metabolism of at least one drug in a subject in need thereof. More specifically, the method comprising the step of administering to the subject an effective amount of at least one of, the system disclosed herein, the modified bacterial cell/s or cell populations, any compositions or kits thereof, or any combinations thereof with at least one drug. More specifically, in some embodiments, the treated subject is administered with at least one of: (I), at least one drug metabolism modulatory system comprising at least one of: (a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and (b) at least one selective component comprising at least one protospacer, wherein said at least one protospacer is targeted by at least one spacer of the CRISPR array of (a), to inactivate said selective component. Alternatively, or additionally, the treated subject is administered with (II), at least one bacterial cell and/or a population of the bacterial cells, or any composition or product thereof, exhibiting modified metabolism of the at least one drug. Alternatively, or additionally, the treated subject is administered with (III), at least one composition, kit or system comprising at least one of (I) and (II); and (IV) any combination of said at least one of (I), (II) and (III) with said at least one drug.

Still further, in a further aspect, the invention provides a method for treating, preventing, ameliorating, reducing or delaying the onset of at least one pathologic disorder in a subject in need thereof. More specifically, the method comprising the step of administering to the subject an effective amount of at least one of, the system disclosed herein, the modified bacterial cell/s or cell populations, any compositions or kits thereof, or any combinations thereof with at least one drug. More specifically, in some embodiments, the treated subject is administered with at least one of: (I), at least one drug metabolism modulatory system comprising at least one of: (a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and (b) at least one selective component comprising at least one protospacer, wherein said at least one protospacer is targeted by at least one spacer of the CRISPR array of (a), to inactivate said selective component. Alternatively, or additionally, the treated subject is administered with (II), at least one bacterial cell and/or a population of the bacterial cells, or any composition or product thereof, exhibiting modified metabolism of the at least one drug. Alternatively, or additionally, the treated subject is administered with (III), at least one composition, kit or system comprising at least one of (I) and (II); and (IV) any combination of said at least one of (I), (II) and (III) with said at least one drug.

These and other aspects of the present disclosure will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. The Drug metabolism modulatINg (DOIN) component

Figure shows an embodiment for the drug metabolism modulating, GusR K125A vector introduce the K125A mutant GusR repressor into the targeted bacterial cell. Ori-origin of replication, gusR K125A—the gene encodes the GusR mutant enzyme, Cas-Cas genes, CRISPR array-comprise alternating conserved repeats and spacers; in red-spacer that target the selective vector.

FIG. 2. The selective vector introduces bacterial toxin into the targeted bacterial cell

Figure shows an embodiment for the selective component. Ori-origin of replication, Toxin-the gene encodes toxin that kills bacteria, Proto-spacer-the region on the selective vector that is targeted by the cognate spacer resides in the DOIN vector.

FIG. 3A-3C. Modulation of the GUS operon by the GusR mutant K125A

Schematic illustration of the E. coli GUS operon. The GUS operon is comprised of three genes: gusA, gusB and gusC. The gusR gene encodes the operon repressor GusR.

FIG. 3A: In the absence of a ligand, the GusR binds its upstream regulatory region (operator) and inhibits the expression of the operon genes.

FIG. 3B: In its presence, a ligand (circles) binds to the GusR repressor, which results in conformational change and the release of the repressor from the operator. This leads to the expression of the GUS operon.

FIG. 3C: In the presence of the GusR_K125A mutant (also denoted by the amino acids sequence of SEQ ID NO: 9, dark arrow with a star, also referred to herein as TBX201), the GusR remains attached to the regulatory region, even in the presence of a ligand, and no GUS enzyme is produced.

Circles-GUS-inducing ligand; star-mutation K125A in the GusR protein.

FIG. 4. Inhibition of the GUS activity using the GusR K125A mutant in bacterial strains

The histogram presents GUS specific activity (arbitrary units) measured for the mutant repressor K125A in two bacterial hosts, BW25113 and MG1655. The activity of the GUS operon was determined in the absence (−) or presence (+) of the GUS operon inducer PNPG. Data represent at least two independent experiments.

FIG. 5. GusR K125A mutant inhibits the GUS enzymes activity in additional E. coli strains

The specific activity of the GUS enzyme (arbitrary units) was determined according to the GUS assay. For each strain, specifically, the MG1655 (grey bars) and Nissle1917 (black bars), two cultures were used: one harboring an empty vector (vector) and second harboring the DOIN vector (the modulatory component of the present disclosure) expressing the GusR mutant K125A (K125A). The cultures were incubated in the presence or absence of PNPG, which was used as substrate for the GUS enzyme (GUS substrate). Data represent at least two independent experiments.

FIG. 6. Transduction of the modulatory component into different microbiome isolates Transductions (TFU/ml) of the DOIN vector into microbiome isolates was determined using the TFU assay, as described previously. Test bacteria culture (MG1655 (grey bars), BW25113 (white bars)) was grown to reach mid-exponential growth phase and incubated for 1 hr with a lysate of transducing particles (modified bacteriophages) containing the DOIN vector expressing GusR K125A_CRISPR, used as the modulatory component. Ten-fold serial dilutions were plated in duplicates on selective plates, which only bacteria that were transduced were able to grow on. Dashed grey horizontal line indicates the assay limit of detection. Data represent at least two independent experiments.

FIG. 7. GusR R73 and GusR Y164F mutants affect the GUS enzyme activity

The specific activity of the GUS enzyme (arbitrary units) was determined according to the GUS assay, in E. coli strains BW25113 (white bars) and MG1655 (grey bars), that were transduced with the DOIN vector encoding the GusR R73 and GusR Y164F mutants. For each strain, three cultures were used, one harboring an empty vector (vector) and the second and third harboring the DOIN vector, expressing either the GusR R73A mutant or the Y164F mutant, respectively. The cultures were incubated in the presence or absence of PNPG, which was used as substrate for the GUS enzyme (GUS substrate). Data represent at least two independent experiments.

FIG. 8A-8C. Conversion of bacterial cell population in-vitro culture

FIG. 8A. A schematic Illustration of an embodiments of the platform of the present disclosure that comprises two different components, the modulatory component, and the selective component, each in a delivery vehicle that is modified bacteriophage. Both phages have an identical structure and target the same bacteria but differ in their payloads. Left-the modulating component (marked as “CRISPR”) comprise nucleic acid sequence encoding the GusR mutant K125A and a CRISPR-Cas3 system (DOIN vector); Right-(marked as “Selective”), is phage having a modified genome of non-replicating T7 lytic phage. These phages act sequentially. The CRISPR array of the modulatory component specifically targets the “Selective” phages and rescues any bacteria that contains this dual GusR mutant-CRISPR modulating component from the selective component.

FIG. 8B. Bacterial culture of E. coli BW25113 isolate was grown until reached mid-exponential growth phase (10⁷ CFU/ml). At time point 0 hr. “CRISPR” phages containing the DOIN vector were used to infect the culture at an MOI of ˜0.1. One hour later, at time point 1 hr, and again at time point 24 hr, the “Selective” component phages were used to infect the culture at an MOI of ˜10. The proportion of GUS-expressing (light gray) and GUS-inhibited bacteria (dark gray) from the total bacterial population (% bacteria) was determined. The illustrated phages with arrows indicate the timepoint of infection. Data are representative of two independent experiments. FIG. 8C. Samples from the bacterial culture were taken at timepoint 0 hr (before the addition of the “CRISPR” phages) and at timepoint 48 hr and the GUS activity was measured.

FIG. 9. GusR mutant K125A reduces the cytotoxicity of SN38

The figure shows that GusR mutant K125A reduces the cytotoxicity of SN38 by inhibiting the metabolic conversion of the deactivated SN38G into SN38 by bacterial GUS, in a substrate concentration-dependent manner. Two BW25113 bacterial cultures were used: Vector bacteria expressing empty vector, and K125A bacteria containing the DOIN vector expressing GusR mutant repressor K125A. The cultures were incubated with PNPG to induce the GUS operon and were then incubated with the indicated SN-38G (SantaCruz, 212931A) concentrations, followed by lysis of the bacteria. HL29 (ATCC® HTB-38™) cells were maintained according to the manufacture instructions and incubated for 48 hr with the different bacterial extractions. The viability of the cells, incubated with the bacterial lysates, were determined using the XTT viability assay, in three independent biological repeats using four replicates.

FIG. 10. Cytotoxicity of SN38 is reduced due to reduction in SN38G metabolism by GUS

The histogram shows cell viability in various treatment groups. GusR mutant K125A reduces the cytotoxicity of SN38 by inhibiting the metabolism of SN38G by the bacterial GUS, in an enzyme concentration-dependent manner. The study was performed as described in FIGURE, with the following modifications: The cultures were incubated with PNPG to induce the GUS operon, diluted as indicated, followed by incubation with 1 μM of SN-38G, and subsequently, lysis of the bacteria. The viability of the cells was determined as above.

### DETAILED DESCRIPTION OF THE INVENTION

The present disclosure relates to manipulation of the microbiome to modulate metabolism of drugs and other substances. Such modulation results in various effects, for example, reduced toxicity of a drug, improved drug adsorption, modulated stability, and increased drug availability and uptake and activation of pro-drugs. For example, phage-based transducing particles as disclosed herein may be synthetically engineered and the host range may be pre-designed and defined. This feature enables the determination of the targeted bacterial population, e.g., enteric bacterial population, in a precise and distinct form. The systems disclosed herein provide at least two components, as exemplified herein as a non-limiting embodiment, these two components may be two types of phage-based transducing particles, one type comprises transducing particles that comprise and deliver drug metabolism modulating component (also referred to herein as Drug metabolism modulatINg (DOIN)) vector, or the modulating component of the present disclosure, that further comprises the CRISPR-Cas system, or module, and transducing particles that pack and/or deliver the selective vector to the target cell/s. The selective vector comprises nucleic acid sequence which encode toxic products that kill, attenuates, disrupts or inhibit bacterial growth, and/or function; and at least one proto-spacer that its sequence matches at least one spacer, and is therefore targeted by at least one spacer in the CRISPR array comprised in the modulatory component (DOIN vector). This system enables modulation of drug metabolism and sustained enrichment of the bacterial population holding the modulatory component (DOIN vector or delivery vehicle) and the CRISPR in it. The DOIN vector comprises nucleic acid sequence encoding at least one component that modulate drug metabolism. This component is delivered to improve the activity of specific drug consumed by an individual or to lower the toxic side effects accompanied the use of this drug. The CRISPR-Cas module of the DOIN vector/s comprises at least one spacer that targets the matching proto-spacer in the selective component vector (delivery vehicle). The DOIN vector is delivered into the targeted bacterial population, e.g., in the gut. Once delivered, the transduced nucleic acid sequence of the delivery vehicle is expressed and produces the active component involved in drug metabolism, and thus provides a modulatory means. The introduction of this component modifies the capabilities of the existing bacterial population in the gut, thereby manipulating the activity of such microbiome cell population (e.g., bacterial population). Once expressed in the targeted microbiome-microorganisms, specifically, targeted bacteria, the abovementioned element is active and functional. In some embodiments, following the introduction of the DOIN vector into microbiome hosts (bacterial host cells), the selective component is then introduced. The introduction of the selective component results in selective killing of the targeted bacterial hosts. However, cells that hold the DOIN vector are protected from killing of the selective vector, since the CRISPR on the DOIN vector targets and eliminate the selective vector. The end result of the active selection of DOIN vector-harboring bacteria described above is the creation of selective pressure that favors bacterial host cells that hold and produce the DOIN vector.

Thus, a first aspect of the present disclosure relates to a system for modulating drug metabolism by modifying microorganisms of the microbiome, for example, bacterial cell/s and/or bacterial cell population. The target cells, for example, bacterial cell or cell population modified by the system of the present disclosure exhibits modified metabolism of at least one drug. More specifically, the system of the present disclosure comprises at least two elements, or components.

One component, element or part (a) of the disclosed system, comprises at least one drug metabolism modulating component. This component comprises at least one nucleic acid sequence encoding and/or modulating at least one element participating in metabolism of at least one drug, at least one cas gene and at least one Clustered, Regularly Interspaced Short Palindromic Repeat (CRISPR) array. The second component, element or part of the system disclosed herein (b), comprises at least one selective component comprising at least one protospacer. It should be noted that the at least one protospacer of the selective component is targeted by at least one spacer of the CRISPR array of the modulatory component (a), thereby inactivating the selective component. It should be appreciated that the disclosed system may be also referred to herein as a kit, comprising the disclosed at least two components.

The systems disclosed herein are effective in modulating metabolism of drugs. More specifically, the metabolism of a drug is modulated by the drug metabolism modulatory component of the disclosed systems. Drug metabolism as used herein, refers to the metabolic breakdown of drugs by living organisms, usually through specialized enzymatic systems. More generally, xenobiotic metabolism is the set of metabolic pathways that modify the chemical structure of xenobiotics, which are compounds foreign to an organism's normal biochemistry, such as any drug or poison. These reactions often act to detoxify poisonous compounds although in some cases the intermediates in xenobiotic metabolism can themselves cause toxic effects. The study of drug metabolism is called pharmacokinetics. The metabolism of pharmaceutical drugs is an important aspect of pharmacology and medicine. For example, the rate of metabolism determines the duration and intensity of a drug's pharmacologic action. Although metabolism typically inactivates drugs, some drug metabolites are pharmacologically active, sometimes even more so than the parent compound. An inactive or weakly active substance that has an active metabolite is called a prodrug, especially if designed to deliver the active moiety more effectively.

Drugs can be metabolized by oxidation, reduction, hydrolysis, hydration, conjugation, condensation, or isomerization; whatever the process, the goal is to make the drug easier to excrete. The enzymes involved in metabolism are present in many tissues but generally are more concentrated in the liver. For many drugs, metabolism occurs in 2 phases. Phase I reactions involve formation of a new or modified functional group or cleavage (oxidation, reduction, hydrolysis); these reactions are non-synthetic. Phase II reactions involve conjugation with an endogenous substance (e.g., glucuronic acid, sulfate, glycine); these reactions are synthetic. Metabolites formed in synthetic reactions are more polar and thus more readily excreted by the kidneys (in urine) and the liver (in bile) than those formed in non-synthetic reactions. Some drugs undergo only phase I or phase II reactions; thus, phase numbers reflect functional rather than sequential classification. The most important enzyme system of phase I metabolism is cytochrome P-450 (CYP450), a microsomal superfamily of isoenzymes that catalyzes the oxidation of many drugs. The electrons are supplied by NADPH-CYP450 reductase, a flavoprotein that transfers electrons from NADPH (the reduced form of nicotinamide adenine dinucleotide phosphate) to CYP450. CYP450 enzymes can be induced or inhibited by many drugs and substances resulting in drug interactions in which one drug enhances the toxicity or reduces the therapeutic effect of another drug. For examples of drugs that interact with specific enzymes. Glucuronidation, the most common phase II reaction, is the only one that occurs in the liver microsomal enzyme system. Glucuronides are secreted in bile and eliminated in urine. Thus, conjugation makes most drugs more soluble and easily excreted by the kidneys. Amino acid conjugation with glutamine or glycine produces conjugates that are readily excreted in urine but not extensively secreted in bile.

Still further, in some modulation of drug metabolism as used herein refers to either increase or decrease of the action of various metabolizing enzymes that may either activate the drug by increasing the activity, affinity, stability, bioavailability, efficacy, solubility, of the drug, or alternatively, deactivate the drug by decreasing the activity, affinity, stability, bioavailability, efficacy, solubility, of the drug.

Still further, modulation of the metabolism of drugs by the disclosed systems or kits as used herein, encompasses any change, induction, increase and elevation, or alternatively, decrease, reduction and attenuation of at least one of the activity, affinity, stability, bioavailability, efficacy, solubility, of the drug, or alternatively, deactivate the drug by decreasing the activity, affinity, stability, bioavailability, efficacy, solubility, of the drug. More specifically, “Modulation” as used herein means a perturbation of function and/or activity, stability and/or structure, affinity, stability, bioavailability, efficacy, solubility, of the drug. In certain embodiments, modulation may involve an increase or decrease in drug metabolism, thereby leading to the desired modulation of the function and/or activity, stability and/or structure, affinity, stability, bioavailability, efficacy, solubility, of the drug. More specifically, as used herein “increasing”, “increased”, “increase”, “enhance” or “activate” are all used herein to generally mean an increase by a statistically significant amount; for the avoidance of any doubt, the terms “increased”, “increase”, “enhance” or “activate” means an increase of at least 10% as compared to a reference level of the function and/or activity, stability and/or structure, affinity, stability, bioavailability, efficacy, solubility, of the drug. For example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level, of the function and/or activity, stability and/or structure, affinity, stability, bioavailability, efficacy, solubility, of the drug. Alternatively, as used herein “inhibition”, “moderation”, “reduction”, “decrease” or “attenuation”, “prevention”, “suppression”, “repression”, “elimination” are all used herein to generally mean a decrease by a statistically significant amount; for the avoidance of any doubt, the terms “inhibition”, “moderation”, “reduction”, “decrease” or “attenuation”, “prevention”, “suppression”, “repression”, “elimination” means a decrease of at least 10% as compared to a reference level of the function and/or activity, stability and/or structure, affinity, stability, bioavailability, efficacy, solubility, of the drug. For example a decrease of at least about 20%, or at least about 30%, or at least about 40%, or at least about 50%, or at least about 60%, or at least about 70%, or at least about 80%, or at least about 90% or up to and including a 100% increase or any decrease between 10-100% as compared to a reference level, or at least about a 2-fold, or at least about a 3-fold, or at least about a 4-fold, or at least about a 5-fold or at least about a 10-fold decrease, or any decrease between 2-fold and 10-fold or greater as compared to a reference level, of the function and/or activity, stability and/or structure, affinity, stability, bioavailability, efficacy, solubility, of the drug.

Thus, in some embodiments, the drug metabolism component of the present disclosure is composed of any element that modulates the metabolism of the drug in a manner that either increase and/or decrease at least one of the function and/or activity, stability and/or structure, affinity, stability, bioavailability, efficacy, solubility, of the drug.

It should be appreciated that throughout the present specification the terms “drug metabolism modulating component”, “Drug metabolism modulatINg (DOIN)”, “modulatory component”, “modulating component” are used interchangeably by the present disclosure, and all refer to a component, that in some embodiments comprises nucleic acid molecule that encodes and/or modulates at least one element participating in metabolism of at least one drug, at least one cas gene and at least one CRISPR array.

It should be understood that specifically in cases where the nucleic acid sequence of the drug metabolism modulating component of the systems disclosed herein modulates at least one target element participating in metabolism of the target drug, such nucleic acid sequence may be any modulatory nucleic acid sequence. Examples for modulating nucleic acid sequences may include but are not limited to siRNAs, miRNA, dsRNA, antisense oligo, gRNA that recruits nucleases (e.g., Cas/CRISPR system) and the like, that may enhance, reduce, or modify directly or indirectly the target element participating in metabolism of the target drug. Alternatively, the at least one nucleic acid sequence provided with the modulatory component may encode any element that enhances, repressed, activates and/or inactivates the target element hat participates in the metabolism of the target drug. In some embodiments, the CRISPR system of the modulating component is further used not only to provide an absolute linkage between the selective component and the modulating component, but also to directly modulate the target element participating in metabolism of the target drug, e.g., either to eliminate (knockdown, e.g., by cutting and/or destructing the target nucleic acid sequence, or by recruiting inhibitory elements, such as transcription repressors) the target element, or to enhance and elevate this component (e.g., where the CRISPR system recruits activators, for example transcription activators). Thus, in such particular and no-limiting embodiments, the at least one nucleic acid sequence encoding or modulating at least one element participating in metabolism of the target drug is comprised within the CRISPR array (e.g., spacers that specifically target the target element participating in metabolism of the target drug).

Still further, in some embodiments of the system disclosed herein, the modulating component is comprised within at least one delivery vehicle that specifically targets the cell/s and/or the cells in a target cell population, for example, bacterial cell population. Such delivery vehicle is therefore compatible to the desired target cells. It should be understood that the delivery vehicles or vectors, specifically, the bacteriophage-based transducing particles that comprise the components of the present disclosure, specifically, the modulatory component and/or the selective component as disclosed herein, are compatible to the target cell, for example, the bacterial cell. Thus by the term “compatible” in the context of the present disclosure it is meant that a particular delivery vehicle may comprise a specific suitable host recognition element that enables recognition with the specific target cell. “Recognition” as used herein also encompasses binding, attachment, absorption, penetration of the delivery vehicle into the target cell.

In yet some further embodiments, the selective component of the system of the present disclosure is comprised within at least one delivery vehicle, that specifically targets the target cell/s and/or the target cells in a cell population. Still further, in some embodiments, the selective component may comprise at least one agent that affects cell viability and/or activity.

“Selective component” as used herein, refers to an element or component of the system of the invention that enables, facilitates, leads to, and acts on, selecting, choosing, electing or enriching a specific population of the target host cells, for example, the target bacterial cells, specifically, a population of cells that carry the cas-CRISPR system of the present disclosure. More specifically, a population of bacterial cells that carry the drug metabolism modulating component of the invention, that also contain spacers in the CRISPR array that are directed against the protospacer in the selective component. Thus, the selective component provides selective advantage to the desired population, for example by imposing conditions that enable and allow only the survival of the selected desired population (in specific embodiments, any population or cells that carry the drug metabolism modulating component of the invention). As indicated herein, the selective component of the present disclosure comprises a toxic element that kill, inhibit, or reduces the target cells (e.g., bacterial cells) growth, viability and/or function. It should be appreciated that the terms “inhibition”, “moderation”, “reduction”, “decrease” or “attenuation”, “prevention”, “suppression”, “repression”, “elimination” as referred to herein, relate to the retardation, restraining or reduction of a process (e.g., growth, viability and/or function) by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

Moreover, at least one spacer of the CRISPR array of the invention may encode a sequence sufficiently complementary to a nucleic acid sequence (or a proto-spacer) comprised as the nucleic acid sequence of interest within the selective component of the invention or any systems thereof, so as to target and inactivate the selective component, for example, by cutting the target nucleic acid sequence that comprise the protospacer using the Cas nuclease. In some embodiments, “inactivate” means delay, decrease, inhibit, eliminate, attenuate or stop the activity of the selective component. It should be noted that such inactivation renders a bacterium comprising the drug metabolism modulating component insensitive and resistant to the selective component of the invention or systems thereof. It should be appreciated that sufficient complementarity as used herein reflects any complementarity of between about 10% to 100%, more specifically, complementarity of about 10%, 15%, 20%, 25%, 30%, 35%, 40%, 45%, 50%, 65%, 70%, 75%, 80%, 85%, 90%, 95%, 96%, 97%, 98%, 99% and 100%. In certain embodiments, “Complementarity” refers to a relationship between two structures each following the lock-and-key principle. In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G).

In some embodiments, the at least one delivery vehicle used for the modulating component, as well as for the selective component is, or comprises at least one genetic element. It should be understood that the delivery vehicle used for these at least two components may be either identical, or different. In yet some further embodiments, both delivery vehicles must target and transduce the modulatory component or the selective component to the same target cell/s. In some non-limiting embodiments, such genetic element may be at least one of: at least one transducing particle, at least one bacteriophage, bacteriophage-based, bacteriophage-like transducing particles and/or at least one modified bacteriophage, or any fragments or parts thereof, or at least one vector, plasmid and/or construct that comprises and/or encodes either the modulating component and/or the selective component, and any combinations or cocktails thereof.

In yet some further embodiments, the delivery vehicle used for the modulatory component and/or the selective component/s of the system of the is at least one bacteriophage or any bacteriophage-like transducing particle.

“Vehicles” or “delivery Vehicles” as used herein encompass vectors or any transducing particles such as bacteriophage, bacteriophage-like particles, plasmids, phagemides, viruses, integratable DNA fragments, and other vehicles, which enable the transfer of nucleic acid molecules into a desired target cell, and in some further embodiments, leads to expression of the transduced nucleic acid molecule in the target cell. A transducing particle or element as used herein refers to any vector or vehicle capable of transducing and inserting nucleic acid molecule or any cargo into a target cell, or an artificial cellular system.

Vectors are typically self-replicating DNA or RNA constructs containing the desired nucleic acid sequences, and operably linked genetic control elements that are recognized in a suitable cell and effect the translation of the desired gene. Generally, the genetic control elements can include a prokaryotic promoter system. Such system typically includes a transcriptional promoter, transcription enhancers to elevate the level of RNA expression. Vectors usually contain an origin of replication that allows the vector to replicate independently of the cell.

Accordingly, the term control and regulatory elements includes promoters, terminators and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operatively linked to it may be used in these vectors to express DNA sequences encoding any desired protein using the method of this invention.

A vector or delivery vehicle may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988). It is to be understood that this definition of delivery vehicle/s is relevant to any system, method or composition as described in the present disclosure.

Still further, in some embodiments, when plasmid/s or other construct/s cassettes or other genetic elements are used as a delivery vehicle, they comprise nucleic acid sequences encoding the modulating component, or alternatively, the toxic component that inhibits and/or reduces cell viability, and/or function.

In yet some further embodiments, the delivery vehicle of the present disclosure may be particularly suitable for specific target cells, for example, for a specific bacterial cells, such that the modulatory component and/or the selective component of the disclosed systems will be introduced or transduced predominantly to the target cells (e.g., specific bacterial cell type in the microbiome). Still further in some embodiments, of the selective components of the disclosed systems, the delivery vehicle may be at least one bacteriophage or any bacteriophage-like, or bacteriophage-based transducing particle, that comprises and/or encodes at least one toxic element that kill bacterial cells or disrupt, attenuate, and/or inhibit bacterial growth and/or function. This toxic element may be provided by, comprised within and/or transduced by any genetic element, for example, a plasmid, a construct, transposon, bacteriophage, modified bacteriophage, phage-based. In some embodiments, the selective element may be provided as a lytic phage.

Under the term “bacteriophage” it is meant a virus that infects, replicates and assembles within prokaryotes, such as bacteria. It should be noted that the term “bacteriophage” is synonymous with the term “phage”. Phages are composed of proteins that encapsulate a DNA or RNA genome, which may encode only a few or hundreds of genes thereby producing virions with relatively simple or elaborate structures. Phages are classified according to the International Committee on Taxonomy of Viruses (ICTV) considering morphology and the type of nucleic acid (DNA or RNA, single- or double-stranded, linear or circular). About 19 phage families have been recognized so far that infect bacteria and/or archaea (a prokaryotic domain previously classified as archaebacteria). Many bacteriophages are specific to a particular genus or species or strain of cell.

It should be appreciated that any suitable phage may be used as the delivery vehicle by the methods, systems and compositions of the present disclosure.

In some non-limiting embodiments the bacteriophage of the presently disclosed subject matter belongs to the order Caudovirales (for example to the family of Podoviridae, Myoviridae or Siphoviridae) or to the order of Ligamenvirales (for example to the family of Lipothrixviridae or Rudivirus). Phages from other families are also encompassed by the present disclosure, for example Ampullaviridae, Bicaudaviridae, and Clavaviridae to name but few.

In other embodiments the bacteriophage according to the present disclosure is one of (but not limited to) the bacteriophage family Podoviridae, Myoviridae or Siphoviridae, Lipothrixviridae or Rudivirus.

In certain specific embodiments, the bacteriophage according to the present disclosure is at least one of T7 like-virus or T4 like-virus.

In further specific embodiments, the phage used as the delivery vehicle by the methods of the invention, systems and compositions of the disclosure described herein after, may be a T7-like-virus, specifically, Enterobacteria phage T7. Bacteriophage T7 are DNA viruses having a lytic life cycle.

More specifically, the phage according to the present disclosure may be Escherichia coli phage T7 (a member of the Podoviridae family of the Caudovirales (tailed phages) order, as detailed above). T7 is composed of an icosahedral capsid with a 20-nm short tail at one of the vertices. The capsid is formed by the shell protein gene product (gp) 10 and encloses a DNA of 40 kb. A cylindrical structure composed of gp14, gp15, and gp16 is present inside the capsid, attached to the special vertex formed by the connector, a circular dodecamer of gp8 (8, 10). The proteins gp11 and gp12 form the tail; gp13, gp6.7, and gp7.3 have also been shown to be part of the virion and to be necessary for infection, although their location has not been established. The main portion of the tail is composed of gp12, a large protein of which six copies are present; the small gp11 protein is also located in the tail. Attached to the tail are six fibers, each containing three copies of the gp17 protein.

Phages used as the delivery vehicle by the methods, systems and compositions of the present disclosure may include other groups members of the family Podoviridae, for example but not limited to T3 phages, Φ29, P22, P-SPP7, N4, ε15, K1E, K1-5 and P37.

In some specific embodiments, phages used as the delivery vehicle by the methods, systems and compositions of the present disclosure may include, but are not limited to Enterobacteria phage T7, Enterobacteria phage 13a, Yersinia phage YpsP-G, Enterobacteria phage T3, Yersinia phage YpP-R, Salmonella phage phiSG-JL2, Salmonella phage Vi06, Pseudomonas phage gh-1, Klebsiella phage K11, Enterobacter phage phiEap-1, Enterobacter phage E-2, Klebsiella phage KP32, Klebsiella phage KP34, Klebsiella phage vB_KpnP_KpV289 and Pseudomonas phage phiKMV.

By way of another example, the bacteriophage/s include, but are not limited to, those bacteriophage capable of infecting a bacterium including but not limited to any one of the proteobacteria, Firmicutes and Bacterioidetes phyla.

By way of further examples, the bacteriophage that may be useful as a delivery vehicle for the disclosed components, or alternatively, as a source for heterologous host recognition elements for a delivery vehicle suitable to the target cells (e.g., bacterial cells), include but are not limited to, those bacteriophage capable of infecting bacteria belonging to the following genera: Escherichia coli, Pseudomonas, Streptococcus, Staphylococcus, Salmonella, Shigella, Clostidium, Enterococcus, Klebsiella Acinetobacter and Enterobacter.

To name but few, these bacteriophages, may include but are not limited to bacteriophages specific for Staphylococcus aureus, specifically, at least one of vB_Sau. My D1, vB_Sau My 1140, vB_SauM 142, Sb-1, vB_SauM 232, vB_SauS 175, vB_SauM 50, vB_Sau 51/18, vB_Sau.M. 1, vB_Sau.M. 2, vB_Sau.S. 3, vB_Sau.M. 4, vB_Sau.S. 5, vB_Sau.S. 6, vB_Sau.M.7, vB_Sau.S.8, vB_Sau.S.9, vB_Sau.M.10, vB_Sau.M.11. In yet some further embodiments, bacteriophages specific for Klebsiella pneumoniae, may be also applicable for the present invention. In more specific embodiments, these phages may include vB_Klp 1, vB_Klp 2, vB_Klp. M.1, vB_Klp. M.2, vB_Klp. P.3, vB_Klp. M.4, vB_Klp. M.5, vB_Klp. M.6, vB_Klp. 7, vB_Klp. M.8, vB_Klp. M.9, vB_Klp. M.10, vB_Klp. P.11, vB_Klp. P.12, vB_Klp. 13, vB_Klp. P.14, vB_Klp. 15, vB_Klp. M.16. Still further, in certain embodiments, bacteriophages specific for Pseudomonas aeruginosa, may be applicable as the delivery vehicles of the invention or alternatively, as a source for heterologous host recognition elements for a suitable delivery vehicle. Non-limiting examples for such bacteriophages include but are not limited to vB_Psa.Shis 1, vB_PsaM PAT5, vB_PsaP PAT14, vB_PsaM PAT13, vB_PsaM ST-1, vB_Psa CT 27, vB_Psa CT 44 K, vB_Psa CT 44 M, vB_Psa 16, vB_Psa Ps-1, vB_Psa 8-40, vB_Psa 35 K, vB_Psa 44, vB_Psa 1, vB_Psa 9, vB_Psa 6-131 M, vB_Psa cr 37, vB_Psa CT 45 S, vB_Psa CT 45 M, vB_Psa CT 16 MU, vB_Psa CT 41, vB_Psa CT 44 MU, vB_Psa CT 43, vB_Psa CT 11 K, vB_Psa 1638, vB_Psa Ps-2, vB_Psa 35 CT, vB_Psa 35 M, vB_Psa S.Ch.L, vB_Psa R1, vB_Psa SAN, vB_Psa L24, vB_Psa F8, vB_Psa BT-4, vB_Psa BT-2 (8), vB_Psa BT-1 (10), vB_Psa BT-4-16, vB_Psa BT-5, vB_Psa F-2, vB_Psa B-CF, vB_Psa Ph7/32, vB_Psa Ph7/63, vB_Psa Ph5/32, vB_Psa Ph8/16, vB_Psa Ph11/1, vB_Psa, vB_Psa 3, vB_Psa 4, vB_Psa 5, vB_Psa 6, vB_Psa 7, vB_Psa.P. 15, vB_Psa. 17, vB_Psa.M. 18, vB_Psa. 28, vB_Psa.M.2, vB_Psa.M 3, vB_Psa.23, vB_Psa.P. 8, vB_Psa.M. PST7, vB_Psa.M. C5, vB_Psa.M D1038. In further embodiments, bacteriophages specific for Acinetobacter baumanii, may be applicable for the present invention. Such lytuic or temperate phages may include any one of vB_Aba B37, vB_Aba G865, vB_Aba G866, vB_Aba U7, vB_Aba U8, vB_Acb 1, vB_Acb 2. In yet some further embodiments, bacteriophages specific for Enterobacter, may be applicable as the delivery vehicles of the invention or alternatively, as a source for heterologous host recognition elements for a suitable delivery vehicle, specifically, any one of vB_Eb 1, vB_Eb 2, vB_Eb 3, vB_Eb 4 bacteriophages. In yet some further embodiments, Enterococcus faecalis specific bacteriophages may be used. Several non-limiting examples include any one of, vB_Ec 1, vB_Ec 2, vB_Enf.S.4, vB_Enf.S.5 bacteriophages.

In yet some further embodiments, bacteriophages that specifically infect Bacillus anthracis, for example, vB_Bak1, vB_Bak2, vB_Bak6, vB_Bak7, vB_Bak9, vB_BaK10, vB_BaK11, vB_BaK12, vB_BaGa4, vB_BaGa5, vB_BaGa6, may be also applicable for the present invention. Still further, bacteriophages specific for Brucella abortus for example, Tb, vB_BraP IV, vB_BraP V, vB_BraP VI, vB_BraP VII, vB_BraP VIII, vB_BraP IX, vB_BraP X, vB_BraP XII, vB_BraP 12 (b), vB_BraP BA, vB_BraP 544, vB_BraP 141s, vB_BraP 141m, vB_BraP 19s, vB_BraP 19m, vB_BraP 9, bacteriophages specific for Brucella canis, specifically, vB_BrcP 1066, bacteriophages specific for Clostridium perfigenes A.B.C.D.E, for example, vB_CpPI, vB_CpII, vB_CpIII, vB_CpIV, bacteriophages specific for Desulfovibrio vulgaris, specifically, vB_DvRCH1/M1, vB_DvH/P15, vB_DvH/M15, those specific for Enterococcus faecalis, specifically, vB_Ec 1, vB_Ec 2, vB_Enf.S.4, vB_Enf.S.5, bacteriophages specific for Escherichia coli, specifically, vB_Eschc.pod 9, vB_Eschc.Pod 4, vB_Eschc.Shis 7, vB_Eschc.Shis 14, vB_Eschc.Shis 5, vB_Eschc.My 2, PhI-1, PhI-2, PhI3, PhI4, PhI5, T2, T4, T5, DDII, DDVI, DDVII, vB_Eschc.Shis 7/20, vB_Eschc.Shis 1161, vB_Eschc.Shis 8963, vB_Eschc 4, vB_Eschc 11/24, vB_Eschc.Shis 18, vB_Shis 3/14, vB_Sau A, vB_Shis G, vB_Eschc.Shis W, vB_Shis GE25, vB_Eschc.Shis 8962, vB_Eschc 90/25, vB_Eschc 5/25, vB_Eschc 12/25, vB_Eschc H, T3, T6, T7, vB_Eschc 4, vB_Eschc 121, vB_Eschc BaK2, vB_Eschc L7-2, vB_Eschc L7-3, vB_Eschc L7-7, vB_Eschc L7-8, vB_Eschc L7-9, vB_Eschc L7-10, vB_Eschc @8, vB_Eschc.Shis 20, vB_Eschc.Shis 25, vB_Eschc.Shis 27, vB_Eschc.Shis MY, vB_Eschc 11, vB_Eschc 12, vB_Eschc 13, vB_Eschc 17, vB_Eschc 18, vB_Eschc 19, vB_Eschc 20, vB_Eschc 21, vB_Eschc 22, vB_Eschc 23, vB_Eschc 24, vB_Eschc 25, vB_Eschc 26, vB_Eschc 27, vB_Eschc 28, vB_Eschc 29, vB_Eschc 30, vB_Eschc 31, vB_Eschc 32, vB_Eschc 34, vB_Eschc 35, vB_Eschc 37, vB_Eschc 38, vB_Eschc 39, vB_Eschc 44, vB_Eschc 45, vB_Eschc 46, vB_E. coli.M. 1, vB_E. coli.M. 2, vB_E. coli. P.3, vB_E. coli. P.4, vB_E. coli. P.5, vB_E. coli. P.6, vB_E. coli. P.7, vB_E. coli. P.8, phages specific for Salmonella paratyphi, specifically, vB_SPB Diag 1, vB_SPB Diag 2, vB_SPB Diag 3, vB_SPB Diag 3b, vB_SPB Diag Jersey, vB_SPB Diag Beecles, vB_SPB Diag Taunton, vB_SPB DiagB.A.O.R. vB_SPB Diag Dundee, vB_SPBDiagWorksop, vB_SPB Diag E, vB_SPB Diag D. vB_SPB Diag F. vB_SPB Diag H, specific for Salmonella typhi abdominalis vB_Sta Diag A, vB_Sta Diag B1, vB_Sta Diag B2, vB_Sta Diag C1, vB_Sta Diag C2, vB_Sta Diag C3, vB_Sta Diag C4, vB_Sta Diag C5, vB_Sta Diag C6, vB_Sta Diag C7, vB_Sta Diag D1, vB_Sta Diag D2, vB_Sta Diag D4, vB_Sta Diag D5, vB_Sta Diag D6, vB_Sta Diag D7, vB_Sta Diag D8, vB_Sta Diag E1, vB_Sta Diag E2, vB_Sta Diag E5, vB_Sta Diag E10, vB_Sta Diag F1, vB_Sta Diag F2, vB_Sta Diag F5, vB_Sta Diag G, vB_Sta Diag H. vB_Sta Diag J1, vB_Sta Diag J2, vB_Sta Diag K, vB_Sta Diag L1, vB_Sta Diag L2, vB_Sta Diag M1, vB_Sta Diag M2, vB_Sta Diag N, vB_Sta Diag O. vB_Sta Diag T. vB_Sta Diag Vil, vB_Sta Diag27, vB_Sta Diag 28, vB_Sta Diag 38, vB_Sta Diag 39, vB_Sta Diag 40, vB_Sta Diag 42, vB_Sta Diag 46, Salmonella typhimurium, specifically, vB_Stm.My 11, vB_Stm.My 28, vB_Stm.Shis 13, vB_Stm.My 760, vB_Stm.Shis 1, IRA, vB_Stm 16, vB_Stm 17, vB_Stm 18, vB_Stm 19, vB_Stm 20, vB_Stm 21, vB_Stm 29, vB_Stm 512, vB_Stm Diag I, vB_Stm Diag II, vB_Stm Diag III, vB_Stm Diag IV, vB_Stm Diag V. vB_Stm Diag VI, vB_Stm Diag VII, vB_Stm Diag VIII, vB_Stm Diag IX, vB_Stm Diag X. vB_Stm Diag XI, vB_Stm Diag XII, vB_Stm Diag XIII, vB_Stm Diag XIV, vB_Stm Diag XV, vB_Stm Diag XVI, vB_Stm Diag XVII, vB_Stm Diag XVIII, vB_Stm Diag XIX, vB_Stm Diag XX. vB_Stm Diag XXI, vB_Stm Diag 1. vB_Stm Diag 2, vB_Stm Diag 3, vB_Stm Diag 4, vB_Stm Diag 5, vB_Stm Diag 6, vB_Stm Diag 7, vB_Stm Diag 8, vB_Stm Diag 9, vB_Stm Diag 10, vB_Stm Diag 11, vB_Stm Diag 12, vB_Stm Diag 13, vB_Stm Diag 14, vB_Stm Diag 15, vB_Stm Diag 16, vB_Stm Diag 17, vB_Stm Diag 18, vB_Stm Diag 19, vB_Stm Diag 20, vB_Stm Diag 21, vB_Stm Diag 22, vB_Stm Diag 23, vB_Stm Diag 24, vB_Stm Diag 25, vB_Stm Diag 26, vB_Stm Diag 27, vB_Stm Diag 28, vB_Stm Diag 29, vB_Stm Diag 30, vB_Stm Diag 31, vB_Stm Diag 32, vB_Stm Diag 33, vB_Stm Diag 34, vB_Stm Diag 35, vB_Stm Diag 36, vB_Stm Diag 37, vB_Stm Diag 38, vB_Stm Diag 39, vB_Stm Diag 40, vB_Stm Diag 41, vB_Stm Diag 42, vB_Stm Diag 43, vB_Stm Diag 44, vB_Stm Diag 45, vB_Stm Diag 46, vB_Stm Diag 47, vB_Stm Diag 48, vB_Stm Diag 49, vB_Stm Diag 50, vB_Stm Diag 51, vB_Stm Diag 52, vB_Stm Diag 53, vB_Stm Diag 54, vB_Stm Diag 55, vB_Stm Diag 56, vB_Stm Diag 57, vB_Stm Diag 58, vB_Stm Diag 59, vB_Stm Diag 60, vB_Stm Diag 61, vB_Stm Diag 62, vB_Stm Diag 63, vB_Stm Diag 64, vB_Stm Diag 65, vB_Stm. P. 1, vB_Stm. P. 2, vB_Stm. P. 3, vB_Stm. P. 4, Shigella sonnei, specifically, vB_Shs.Pod 3, vB_Eschc.Shis 7/20, vB_Eschc.Shis 1161, vB_Eschc.Shis 8963, vB_Eschc.Shis 8962, vB_Shis GE25, vB_Eschc.Shis W, vB_Shis G, vB_Shis 3/14, vB_Eschc.Shis 18, vB_Shis 1188, vB_Shis 1188 T, vB_Shis 1188 Y, vB_Shis 1188 X, vB_Shis 5514, vB_Shis L7-2, vB_Shis L7-4, vB_Shis L7-5, vB_Shis L7-11, vB_Shis K3, vB_Shis Tul A, vB_Shis Ox2, vB_Shis SCL, vB_Shis Bak C2, vB_Shis 4/1188, vB_Shis 8962, vB_Shis 8963, vB_Shis XIV, vB_Shis 116, vB_Shis 106/8, vB_Shis 20, vB_Shis 90/25, vB_Shis 87/25, vB_Shis 16/25, vB_Shs 7, vB_Shs 38, vB_Shs 92, vB_Shs 1391, vB_Shs. P. 1, vB_Shs. P. 2, vB_Shs. P. 3.

It should be appreciated that in some embodiments, the invention encompasses the use of any of the bacteriophages listed herein and disclosed herein, as well as any variants or hybrids thereof, as delivery vehicles to the components of the present disclosure, specifically, the modulating component and/or the selective component.

In some embodiments, the bacteriophage or bacteriophage-like transducing particle is of the T7-like viruses, the T7-like viruses, or any phage-like particles or transducing particles thereof. In some embodiments, the bacteriophage or bacteriophage-like transducing particle is of the T4-like viruses, the T4-like viruses, or any phage-like particles or transducing particles thereof. In yet some further embodiments, the bacteriophage used as a delivery vehicle may be the Escherichia virus Lambda. It should be however noted that in some embodiments, any bacteriophage, bacteriophage particles or any hybrid bacteriophage or bacteriophage-based article, composed of at least part of sequence/s, either native or modified or mutated sequences, of at least one bacteriophage, and optionally, of two or more different bacteriophages may be used herein as a suitable delivery vehicle for the at least one drug metabolism modulating component of the system of the invention and/or the at least one selective component of the system of the invention.

Still further, it should be appreciated that any delivery vehicles may be used, specifically and phage-based delivery vehicle an any variants thereof. More specifically, “delivery vehicle variants” as herein, may differ one from the other by the content of their nucleic acid sequences and/or by the amino acid sequences of their proteins. Delivery vehicle variants may comprise at least one host recognition element that may be homologous or heterologous, hybrid, native, mutated or any combinations thereof. Specifically, these variants may carry a desirable host recognition element that is compatible with a target cell of interest.

As indicated herein, the delivery vehicle applicable for transducing the modulatory component and/or the selective component as disclosed herein of the disclosed system, are compatible with a target cell, for example, a specific microbiome microorganism, more specifically, gut microbiome bacterial cells. It should be therefore understood that in case phage-based delivery vehicles are used, any native, variant, hybrid, mutated or modified bacteriophage may be used. Such bacteriophage may carry a modified, hybrid or native host recognition element that is compatible with the host cell. In some embodiments the host recognition element may comprise at least one protein, at least two proteins, at least three proteins or more, specifically, structural bacteriophage protein/s, either native or modified, that interact with the host receptor. In some specific embodiments, such structural bacteriophage protein may be a protein/s residing in the tail region of a bacteriophage. As known in the art, in bacteriophages the tail is a protein complex present in the majority of the phages and is involved in host recognition and genome delivery. Two main features are shared by tail structures: tails have a central tubular structure that forms the channel for DNA ejection, which is surrounded by fibers or spikes that are essential in the initial steps of host recognition. For example, the tail of T7 phage is assembled from a dodecamer (i.e. 12 copies) of gp11 (the adaptor) and a hexamer (i.e. 6 copies) of gp12 (the nozzle), onto which six trimers of gp17 attach. T7's six tail fibers attach at the interface between the adaptor and nozzle, thus making contacts with both proteins. The adaptor ring is responsible for the attachment of the preformed tail to the prohead via interactions with the portal composed of 12 subunits of gp8. Bacteriophage components localized at the tail-end of the bacteriophage may be classified as “tail proteins” or “tail-tube proteins” (e.g. referring to gp11 and gp12) and tail fiber (e.g. referring to gp17). As noted above, the host recognition element of the bacteriophage-based delivery vehicle may comprise at least one of these proteins, derived from any of the bacteriophages disclosed by the present disclosure.

Thus bacteriophage components localized at the tail-end of the bacteriophage may be classified as tail proteins (e.g. referring to gp11 and gp12) and tail fiber (e.g. referring to gp17). In specific embodiments the host-recognition element of the phage-based delivery vehicle used in the present disclosure may comprise at least one tail fiber or at least one tail protein.

In some embodiments the at least one protein residing in the tail region of such bacteriophage may be at least one of a tail protein and a fiber protein.

In specific embodiments, the host-recognition element herein described may comprise at least one of gp11, gp12 and gp17, or any combinations thereof. In some specific and non-limiting embodiments, these proteins may be, but not limited to, T7 gp17, gp11 or gp12, any mutant thereof as described herein of or any native or mutated heterologous variants as explained below, or any combination thereof.

Any protein residing in the tail region of any naturally occurring bacteriophage that infects target cells as herein defined is encompassed by the present disclosure, specifically, any host recognition elements compatible with the desired target cell, as well as any combinations thereof.

It should be understood that the delivery vehicle that comprises, or used as, the modulatory or the selective component of the present disclosure may be modified bacteriophages, that may be also referred to herein as “modified particles”, “transducing particles”, “programed transducing particles”, “transducing vehicles”, “vehicles of the invention”, “bacteriophage or phage particles”, “delivery vehicles” and the like.

It should be understood that in some embodiments, where a bacteriophage-based transducing particles (e.g., bacteriophages, or hybrid phages or modified phages), such component may further comprise at least one packaging signal and any other suitable element that facilitates packaging in the delivery vehicle. As noted above, the nucleic acid sequence provided by the invention comprises a packaging signal. The term “packaging signal” as herein defined refers to a nucleotide sequence in e.g. a viral or bacteriophage genome that directs the packaging the of viral or bacteriophage genome into preformed capsids (envelops) during the infectious cycle.

In some embodiments, the systems of the present disclosure target microbiome organism/s in the microbiome cell population. For example, bacterial cells residing within the microbiome. In some embodiments, the cells targeted by the disclosed system/s may be bacterial cell/s and/or cell population. The target bacterial cells are according to some embodiments modified and targeted by the system of the present disclosure, and are, or comprised within gut microbiome cell population. It should be further noted that the cell population comprises at least one bacterium of at least one of, the Proteobacteria, Firmicutes, Bacteroidetes, Actinobacteria, and phyla, or any mutant, variant of isolate or any combination thereof.

In some specific and no-limiting embodiments, the target bacteria or bacterial populations modified by the system disclosed herein are any bacteria of the class Gammaproteobacteria. In yet some further embodiments, the target bacteria or bacterial populations modified by the system disclosed herein are any bacteria of the order Enterobacterales. In yet some further embodiments, the target bacteria or bacterial populations modified by the system/s disclosed herein are any bacteria of the family Enterobacteriaceae.

It should be noted that the Enterobacteriaceae is a large family of Gram-negative bacteria that includes over 30 genera and more than 100 species. Enterobacteriaceae includes, along with many harmless symbionts, many of the more familiar pathogens, such as Salmonella, Escherichia coli, Klebsiella, and Shigella.

Prokaryotic cells according to the present disclosure encompass bacteria cells. The term “bacteria” (in singular a “bacterium”) in this context refers to any type of a single celled microbe. Herein the terms “bacterium” and “microbe” are interchangeable. This term encompasses herein bacteria belonging to general classes according to their basic shapes, namely spherical (cocci), rod (bacilli), spiral (spirilla), comma (vibrios) or corkscrew (spirochaetes), as well as bacteria that exist as single cells, in pairs, chains or clusters.

It should be noted that the term “bacteria” as used herein refers to any of the prokaryotic microorganisms that exist as a single cell or in a cluster or aggregate of single cells. In more specific embodiments, the term “bacteria” specifically refers to Gram positive, Gram negative or Acid-fast organisms. The Gram-positive bacteria can be recognized as retaining the crystal violet stain used in the Gram staining method of bacterial differentiation, and therefore appear to be purple-colored under a microscope. The Gram-negative bacteria do not retain the crystal violet, making positive identification possible. In other words, the term ‘bacteria’ applies herein to bacteria with a thicker peptidoglycan layer in the cell wall outside the cell membrane (Gram-positive), and to bacteria with a thin peptidoglycan layer of their cell wall that is sandwiched between an inner cytoplasmic cell membrane and a bacterial outer membrane (Gram-negative). This term further applies to some bacteria, such as Deinococcus, which stain Gram-positive due to the presence of a thick peptidoglycan layer, but also possess an outer cell membrane, and thus suggested as intermediates in the transition between monoderm (Gram-positive) and diderm (Gram-negative) bacteria. Acid fast organisms like Mycobacterium contain large amounts of lipid substances within their cell walls called mycolic acids that resist staining by conventional methods such as a Gram stain. It should be further appreciated that the present disclosure further encompasses a “cell” any microorganism, specifically, unicellular microorganism that resides in the microbiome or a subject. This term thus refers to most part anaerobic Gram positive and Gram negative strains, as well as to any strain of fungi, protozoa, and archaea. It should be appreciated that the modulating component and/or the selective component of the present disclosure are applicable in modulating drug metabolism of any of the organisms in the microbiome, specifically, the gut microbiome.

It should be however understood that when referring to “cells”, the invention further encompasses in addition to any of the prokaryotic cells exemplified and disclosed by the invention, in some specific embodiments other systems that imitate or mimic cells, artificial cells, vesicles and the like.

In some specific embodiments, the methods of the invention may be used for manipulating, editing and changing the microbiome of a subject in need. More specifically, modulation of the microbiome such that the metabolism of a drug is modulated, as specified herein.

The term “microbiome”, as used herein, refers to the ecological community of commensal, symbiotic, or pathogenic microorganisms in a sample. Examples of microbiomes that can be used with the present disclosure include but are not limited to gut microbiome and oral microbiome, gastrointestinal tract microbiome, skin microbiome, umbilical microbiome, vaginal microbiome, conjunctival microbiome, intestinal microbiome, stomach microbiome, nasal microbiome and urogenital tract microbiome.

In some embodiments, the systems, compositions, kits and methods of the disclosure may be applicable in manipulating the gut microbiome in a subject, for example, a mammalian subject, specifically, a human subject. The term ‘gut microbiome’ (in the colloquial ‘gut flora’) encompasses a complex community of microorganism species that live in the digestive tracts of animals (in this case mammals). In this context gut is synonymous with intestinal and flora with microbiota and microflora. The gut microbiome refers to the genomes of the gut microbiota. Although the mammalian host can most probably survive without the gut flora, the relationship between the two is not merely commensal (a non-harmful coexistence), but rather mutualistic. The mammalian gut microflora fulfill a variety of useful functions, including digestion of unutilized energy substrates, stimulating cell growth, repressing the growth of harmful microorganisms, training the immune system to respond only to pathogens and defending against some diseases. In certain conditions, however, some species are capable of causing disease by producing infection or increasing risk for cancer. Thus, by targeting specific subpopulation of the gut microbiome, the invention provides a therapeutic tailor-made tool for modulating conditions caused by certain microorganisms that are part of the gut microbiome. In some embodiments, such conditions are specifically conditions associate with metabolism of drugs by microorganisms of the gut microbiome.

Composition of the mammalian gut microbiome consists predominantly of bacteria, for the most part anaerobic Gram positive and Gram negative strains, as well as to any one of fungi, protozoa (a phylum or group of phyla that comprises the single-celled microscopic animals, which include amoebas, flagellates, ciliates, sporozoans, and many other forms, belonging to the kingdom Protista), and archaea (constitute a domain of single-celled organisms that lack cell nuclei and are therefore prokaryotes. Archaea were initially classified as bacteria, receiving the name archaebacteria (in the Archaebacteria kingdom). Archaeal cells have unique properties separating them from the other two domains, Bacteria and Eukaryota. Archaea are further divided into multiple recognized phyla. It should be appreciated that the modulating component and/or the selective component of the present disclosure are applicable in modulating drug metabolism of any of the organisms in the microbiome, specifically, the gut microbiome. Thus, in some embodiments, the systems, compositions, kits and methods of the present disclosure target any microorganism of the microbiome, for example, any microorganism of the gut microbiome, in a manner that affects and modulates metabolism of a given drug. Specifically, any of the drugs disclosed by the present disclosure. Non-limiting drugs applicable herein are any of the drugs disclosed by Table 1. Populations of bacterial species vary widely among different individuals, but are relatively constant within an individual over time, some alterations, however, may occur with changes in lifestyle, diet and age. Common evolutionary patterns in the composition of gut microbiome have been observed during life-time of human individuals. Gut microbiome composition and content can change following a long-term diet, and is it also depends on a geographic origin.

More specifically, when referring to composition or content of the human microbiome, or microbiota, is meant a composition with respect to the four predominant phyla of bacteria, namely Firmicutes, Bacteroidetes, Actinobacteria and Proteobacteria, or alternatively with respect to the predominant bacterial genera, namely Bacteroides, Clostridium, Fusobacterium, Eubacterium, Ruminococcus, Peptococcus, Peptostreptococcus and Bifidobacterium. Particularly the Bacteroides, which are the most predominant, may be important for host functioning. Other genera, such as Escherichia and Lactobacillus, although present to a lesser extent, were shown to contribute to host functioning.

Further, of particular relevance to the human gut microbiome is the enterotype classification basing on bacteriological ecosystem, which is independent of age, gender, body weight, or national divisions. There are three human enterotypes: Type 1 is characterized by high levels of Bacteroides (Gram negative); Type 2 has few Bacteroides, but Prevotella (Gram negative) are common; and Type 3 has high levels of Ruminococcus (Gram positive). Enterotypes, however, can be influenced by a long-term diet, for example, people having a high protein and fat diet are predominantly enterotype Type 1 and if changing their dietary patterns to a high carbohydrates diet—in the long-term become enterotype Type 2.

Thus, methods, as well as systems, compositions and kits of the present disclosure pertain to the entire range of bacterial species constituting the mammalian gut microbiome, including qualitative as well as quantitative aspects thereof. They further pertain to less ubiquitous microbiome components, such as of fungi, the known genera include Candida, Saccharomyces, Aspergillus and Penicillium, as well as microorganisms belonging to the domain of Archaea (also Archaebacteria), and further yet unclassified species that cannot be cultured.

It should be understood however that although specifically applicable for human subject in some embodiments, the instant invention also pertains to animal health, particularly mammalian health conditions, as covered by veterinary sciences, and thus provides means to modulate any microbiome population, specifically, gut microbiome organisms to modulate metabolism of a drug administered to any relevant subject as disclosed by the present disclosure.

Still further, in some embodiments, the system of the present invention is designed for modulating drug metabolism by targeted modification of bacterial cell/s and/or bacterial cell population that modulates at least one drug metabolism modulating component. A component that affects at least one of the activity, bioavailability, clearance, stability, toxicity, and absorption of the drug.

In some embodiments, the system disclosed herein is specifically designed to modulate components that directly or indirectly affect drug metabolism. As indicated herein above, the modification of the at least one component may affect at least one of the activity, bioavailability, clearance, stability, toxicity, and absorption of the drug.

In some particular and non-limiting embodiments, the modulation may be the provision of a modulator (e.g., repressor or enhancer) that may either repress metabolism or alternatively, enhance metabolism, and thereby activation or inactivation of a drug by a target enzyme.

In some embodiments, such enzyme is at least one enzyme involved in bacterial energy metabolism. In yet some further embodiments, the enzyme may be specifically an enzyme that participates in carbohydrates metabolism.

In yet some further embodiments, the element that participates in the metabolism of the at least one drug is at least one glucuronide enzyme. Thus, according to these embodiments, the drug metabolism modulating component of the system provided herein, targets and modulates at least one element participating in metabolism of at least one drug. Such element modulated by the modulating component of the present invention may be at least one glucuronide enzyme.

Glucuronide enzymes are produced by mammalian uridine diphosphate-glucuronosyltransferase (UGT) enzymes that append glucuronic acid, to hydroxyl, carboxylate, and other nucleophilic functional groups of aglycones in a process called Glucuronidation. Glycosyl hydrolases are a widespread group of enzymes hydrolyzing the glycosidic bond in carbohydrates or its derivatives. β-glucuronidase is a glycosyl hydrolase which hydrolyses β-glucuronic acid residues from a variety of compounds.

In more specific embodiments, the drug metabolism modulating component of the system disclosed herein, may target, and therefore modulate at least one glucuronide enzyme, that may be at least one β-glucuronidase (GUS) enzyme.

β-Glucuronidase (GUS) enzymes, expressed by the GI microbiota, catalyze the hydrolysis of glycosidic bonds between the glucuronic acid and the glucuronides. Glucuronic acid then enters the Entner-Doudoroff pathway, a bacterial alternative to glycolysis, that catabolizes sugar acids and shunts the resulting pyruvate into the TCA cycle. As a by-product of glucuronide hydrolysis, bacteria regenerate the original molecule that was eliminated by the host, facilitating reuptake by the gut epithelia and recirculation in the bloodstream.

The gene encoding β-glucuronidase, gusA (formerly uidA), which is now widely used as a reporter gene in plants and other organisms, was originally isolated from Escherichia coli. In E. coli, gusA forms part of the GUS operon. There are two genes downstream of gusA, one of which, gusB, encodes a glucuronide-specific permease; the third gene, gusC, function as nonspecific outer-membrane channel. Upstream of gusA, and separately transcribed, is a gene, gusR, encoding a specific repressor of the gus operon. The E. coli GUS is 603 amino acid residues long and it shares about 50% sequence identity with the human GUS with same substrate specificity.

Still further, in some embodiments, GusA as used herein is encoded by the nucleic acid sequence as denoted by SEQ ID NO: 14. In yet some further embodiments, the GusA protein comprises the amino acid sequence as denoted by SEQ ID NO: 15. Still further, in some embodiments, GusB as used herein is encoded by the nucleic acid sequence as denoted by SEQ ID NO: 16. In yet some further embodiments, the GusB protein comprises the amino acid sequence as denoted by SEQ ID NO: 17. Still further, in some embodiments, GusC as used herein is encoded by the nucleic acid sequence as denoted by SEQ ID NO: 18. In yet some further embodiments, the GusC protein comprises the amino acid sequence as denoted by SEQ ID NO: 19. The Non-coding regulatory region of the Gus operon (GusR operator) is denoted by SEQ ID NO: 20.

The asymmetric unit of the E. coli β-glucuronidase structure contains two monomers of 597 ordered residues, and crystallographic symmetry generates the functionally relevant enzyme tetramer. The N-terminal 180 residues resemble the sugar-binding domain of family 2 glycosyl hydrolases, whereas the C-terminal domain (residues 274 to 603) forms an aß barrel and contains the active-site residues Glu413 and Glu504. The region between the N- and C-terminal domains exhibits an immunoglobulin-like β-sandwich domain consistent with other family glycosyl hydrolases.

Still further, in such embodiments, suitable modulating component of the system provided herein may comprise nucleic acid sequence that encodes at least one element that inhibits and/or reduces the expression and/or activity and/or stability of at least one BGUS enzyme.

In some embodiments of the disclosed system/s, the element encoded by the nucleic acid sequence of the modulating component represses the transcription of at least one BGUS enzyme. In yet some further embodiments, the element is a Gus Repressor (GusR), and any variants and mutants thereof.

In yet some specific embodiments, the GusR as used herein is the bacterial GusR.

The crystal structure of the E. coli and S. enterica (members of the Enterobacteriaceae family) GusR protein determined recently, defines a TetR-like ligand-regulated transcriptional repressor. It's a α-helical homodimers, with each monomer composed of DNA-(α1 to α3-) and ligand-binding domains (α4 to α10-54 to 193) (residues 11 to 193 and 54 to 193 respectively in E. coli, see residues in the GusR sequence as denoted by SEQ ID NO: 3).

The E. coli GusR DNA binding domain (DBD) exhibit a helix-turn-helix (HTH) DNA-binding motif that binds to two operator elements, sites 1 and 2. Site 1 is 30 base pairs (bp) in length and is located 200 bp upstream from the GUS operon's ribosome binding site (rbs), while site 2 (40 bp) is only 50 bp from the same rbs. The GusR binding to the DBD is dependent on a conserved TetR family residue (Y49) and specific sequence of amino acids (SCAI) in the HTH motif of the DBD.

The GusR ligand-binding domain in the structures of both E. coli and S. enterica proteins is composed of α-helices (4 to 10). The ligand pocket domain uses hydrogen bond with conserved residues in recognizing the GlcA moiety of a bound ligand. It appears that GusR exhibits species-specific preferences for particular ligands. The affinity for a particular ligand can be adjusted by single-residue changes within the GusR effector-binding pocket.

The active site-adjacent loop 1 (L1) and loop 2 (L2) of the GusR exhibit marked variability in length and amino acid composition and these regions were employed to categorize the GusR proteins, each category contains functionally and cellular specialized GusR. Importantly, the Enterobacteriaceae only encode L1 GUS enzymes.

In yet some further embodiments, the GusR disclosed herein is the Enterobacterial GusR. In more specific embodiments, the GusR referred to herein is the E. coli GusR, of strain BW25113 (DSMZ #27469).

In some embodiments the GusR as used herein is encoded by a nucleic acid sequence comprising the nucleic acid sequence as denoted by SEQ ID NO: 2, or any variants or homologs thereof. Still Further, in some embodiments, the GusR may comprise the amino acid sequence as denoted by SEQ ID NO: 3, or any variants or derivatives thereof.

In yet some further embodiments of the disclosed system, the GusR used in the modulating component of the disclosed system display reduced or abolished affinity to at least one GUS ligand and/or substrate. More specifically, a GusR mutant or variant that display reduced or abolished affinity to any substrate of Gus and thereby, display enhanced repression of Gus, thereby modulating drug metabolism by Gus, may relate to a GusR variant that displays an affinity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced affinity of about 98% to about 100%, as compared to the wild type GusR. More specifically, an enzyme that displays an affinity reduced in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, as compared to the wild type GusR.

Still further, in some embodiments, GusR used in the modulating component of the disclosed system, comprise at least one substitution and/or other modification in residues that form the ligand-binding pocket of the GusR. Such variant is characterized as displaying reduced or abolished affinity to at least one GUS ligand and/or substrate. In yet some further embodiments, any residue/s that form and/or participate either directly or indirectly in ligand binding, for example, the ligand-binding pocket of the GusR can be manipulated to obtain a GusR that display reduced or abolished affinity to at least one GUS ligand and/or substrate, that can be used by the modulatory component of the system of the present disclosure. Still further, specifically in E. coli, the sugar moiety of the bound glucuronide makes six contacts with a total of five GusR residues (H126, K125, R69, E97 and R73). Additional conserved residues in the E. coli GusR, include, but are not limited to Y164, L160, T163 and M87. Thus, in some specific embodiments, a GusR variant and/or mutant that display reduced or abolished affinity to at least one GUS ligand and/or substrate may be any GusR variant having at least one substitution and/or replacement of at least one of: residues H126, K125, R69, E97 and R73, Y164, L160, T163 and M87 of the GusR amino acid sequence as denoted by SEQ ID NO: 3, or any derivatives and variants thereof, or any combinations thereof.

In some particular embodiments of the disclosed systems, GusR comprised within and provided by the modulatory component, carry at least one mutation and/or substitution in at least one of the following residues. In some embodiments, a mutation in the lysine (K) residue in position 125 of the wild type GusR, as denoted by SEQ ID NO: 3, or any derivatives and variants thereof. In yet some further embodiments, the mutation in residue 125 substitutes the lysine (K) residue 125 to Alanine (A). Such mutated GusR is designated herein as K125A GusR. Still further, in some embodiments, the K125A GusR may be encoded by a nucleic acid sequence comprising the nucleic acid sequence as denoted by SEQ ID NO: 8, or any derivatives and variants thereof. In yet some further embodiments, the K125A GusR, may comprise the amino acid sequence as denoted by SEQ ID NO: 9, or any derivatives and variants thereof.

In some embodiments, a mutation in the Tyrosine (Y) residue in position 164 of the wild type GusR, as denoted by SEQ ID NO: 3, or any derivatives and variants thereof. In yet some further embodiments, the mutation in residue 164 substitutes the Tyrosine (Y) residue 164 to Phenylalanine (F). Such mutated GusR is designated herein as Y164F GusR. Still further, in some embodiments, the Y164F GusR may be encoded by a nucleic acid sequence comprising the nucleic acid sequence as denoted by SEQ ID NO: 10, or any derivatives and variants thereof. In yet some further embodiments, the Y164F GusR, may comprise the amino acid sequence as denoted by SEQ ID NO: 11, or any derivatives and variants thereof.

In some embodiments, a mutation in the Arginine (R) residue in position 73 of the wild type GusR, as denoted by SEQ ID NO: 3. In yet some further embodiments, the mutation in residue 73 substitutes the Arginine (R) residue 73 to Alanine (A). Such mutated GusR is designated herein as R73A GusR. Still further, in some embodiments, the R73A GusR may be encoded by a nucleic acid sequence comprising the nucleic acid sequence as denoted by SEQ ID NO: 12. In yet some further embodiments, the R73A GusR, may comprise the amino acid sequence as denoted by SEQ ID NO: 13.

It should be understood that the modulatory component of the disclosed systems may comprise in some embodiments, any combinations of the disclosed GusR mutants, for example, K125A GusR and R73A, K125A GusR and Y164F GusR, R73A GusR and Y164F GusR, and K125A GusR, Y164F GusR, and R73A GusR.

In yet some further embodiments, the at least one drug modulated by the system disclosed herein is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, cns depressant, anticholinergic agent, parasympatholytic agent, immunosuppressant, opioid antidote, anti-asthmatic agent, anti-benign prostatic hyperplasia or anti-gout agent.

In yet some further embodiments, the drug may be at least one antineoplastic agent.

The term “Anti-proliferative therapy” or “anti-neoplastic drug” as used herein refers to any treatment intended for eliminating or destructing (killing) cancer cells or cells of any other proliferative disorder. This includes in some embodiments, “chemotherapeutic drugs or agents” (chemotherapy).

More specifically, “chemotherapeutic drugs or agents” are drugs used to treat cancer and some proliferative diseases. The treatment that uses these drugs intend to stop the growth of cancer cells or other proliferating cells (malignancy and proliferative disease), either by killing the cells or by stopping them from dividing. Chemotherapy may be given by mouth, injection, or infusion, or on the skin, depending on the type and stage of the cancer being treated. It may be given alone or with other treatments, such as surgery, radiation therapy, or biologic therapy. The mechanism underlying the activity of some chemotherapeutic drugs is based on destructing rapidly dividing cells, as many cancer cells grow and multiply more rapidly than normal cells. As a result of their mode of activity, chemotherapeutic agents also harm cells that rapidly divide under normal circumstances, for example bone marrow cells, digestive tract cells, and hair follicles. Insulting or damaging normal cells result in the common side-effects of chemotherapy: myelosuppression (decreased production of blood cells, hence also immuno-suppression), mucositis (inflammation of the lining of the digestive tract), and alopecia (hair loss).

Certain chemotherapy agents have also been used in the treatment of conditions other than cancer and therefore considered as “anti-proliferative”, including ankylosing spondylitis, multiple sclerosis, hemangiomas, Crohn's disease, psoriasis, psoriatic arthritis, rheumatoid arthritis, lupus and scleroderma.

Chemotherapeutic drugs affect cell division or DNA synthesis and function and can be generally classified into groups, based on their structure or biological function. Some chemotherapeutic agents are classified as alkylating agents, anti-metabolites, anthracyclines, plant alkaloids, topoisomerase inhibitors, and other anti-tumor agents such as DNA-alkylating agents, anti-tumor antibiotic agents, tubulin stabilizing agents, tubulin destabilizing agents, hormone antagonist agents, protein kinase inhibitors, HMG-COA inhibitors, CDK inhibitors, cyclin inhibitors, caspase inhibitors, metalloproteinase inhibitors, antisense nucleic acids, triple-helix DNAs, nucleic acids aptamers, and molecularly-modified viral, bacterial or exotoxic agents.

In some optional embodiment, the target drug may be at least one topoisomerase inhibitor. In yet some further embodiments, the target drug may be at least one topoisomerase I inhibitor (TopI). Topoisomerase inhibitors, as used herein, refer compounds that block the action of topoisomerases. DNA topoisomerases (or topoisomerases) are enzymes that catalyze changes in the topological state of DNA, interconverting relaxed and supercoiled forms, linked (catenated) and unlinked species, and knotted and unknotted DNA. Topoisomerase inhibitors may be classified in two broad subtypes: type I topoisomerases (TopI), and type II topoisomerases (TopII). Topoisomerase plays important roles in cellular reproduction and DNA organization, as they mediate the cleavage of single and double stranded DNA to relax supercoils, untangle catenanes, and condense chromosomes in eukaryotic cells. Topoisomerase inhibitors affect these essential cellular processes. Some topoisomerase inhibitors prevent topoisomerases from performing DNA strand breaks while others, deemed topoisomerase poisons, associate with topoisomerase-DNA complexes and prevent the re-ligation step of the topoisomerase mechanism. These topoisomerase-DNA-inhibitor complexes are cytotoxic agents, as the un-repaired single- and double stranded DNA breaks they cause can lead to apoptosis and cell death. As such, Topoisomerase inhibitors are used for treating infectious and cancerous disorders.

As indicated herein, the target drug may be in some embodiments an inhibitor of TopI. TopI as used herein, is a protein that relaxes DNA supercoiling during replication and transcription. Under normal circumstances, TopI attacks the backbone of DNA, forming a transient TopI-DNA intermediate that allows for the rotation of the cleaved strand around the helical axis. TopI then re-ligates the cleaved strand to reestablish duplex DNA. Treatment with TopI inhibitors stabilizes the intermediate cleavable complex, preventing DNA re-ligation, and inducing lethal DNA strand breaks. Camptothecin-derived TopI inhibitors are derived from the tree Camptotheca acuminata, and function by forming a ternary complex with TopI-DNA and are able to stack between the base pairs that flank the cleavage site due to their planar structure. Thus, in some embodiments, the drug targeted by the disclosed systems may be any Camptothecin-derived TopI inhibitors, for example, Camptothecin-11 (CPT-11, Irinotecan).

As shown by the present example, that should be considered as non-limiting embodiments disclosed herein as a proof of concept, the system of the invention is designed to modulate the metabolism of the drug Camptothecin-11 (CPT-11, Irinotecan), or any derivatives thereof. Still further, in some embodiments, non-camptothecins, such as indenoisoquinolines and indolocarbazoles, may be also targeted by the disclosed system. These drugs associate with TopI itself, forming hydrogen bonds with residues that typically confer resistance to camptothecin. Non-CPT inhibitors applicable in the present disclosure may be any one of indenoisoquinoline, phenanthridines, and indolocarbazoles.

It should be however noted that the particular system of the invention may be suitable for modulating the metabolism of any drug that is a substrate of any BGUS enzyme. Particular and non-limiting embodiments of drugs that are inactivated by liver enzymes, for example, by enzymes that perform glucuronidation e.g., UGTs enzymes. Reactivation of these drugs by microbiome microorganisms, for example, bacteria, may lead to undesired effects. Thus, in some embodiments, drugs that are potentially metabolized by the microbiome Gus enzymes are disclosed for example by Table 1, specifically any one of Abacavir, Acemetacin, Acetaminophen, Ambrisentan, Artenimol, Asenapine, Atorvastatin, Axitinib, Bictegravir, Buprenorphine, Canagliflozin, Candesartan, Candesartan cilexetil, Carbamazepine, Chenodeoxycholic acid, Clozapine, Codeine, Cyproheptadine, Dabigatran etexilate, Dapagliflozin, Deferiprone, Delafloxacin, Diclofenac, Diflunisal, Dolutegravir, Eltrombopag, Enasidenib, Entacapone, Epirubicin, Erlotinib, Ertugliflozin, Estradiol, Estradiol acetate, Estradiol benzoate, Estradiol cypionate, Estradiol dienanthate, Estradiol valerate, Etodolac, Etoposide, Ezetimibe, Ezogabine, Flunitrazepam, Flurbiprofen, Fluvastatin, Glecaprevir, Haloperidol, Hydromorphone, Ibuprofen, Imidafenacin, Indacaterol, Indomethacin, Irbesartan, Irinotecan, Isavuconazole, Ketobemidone, Lamotrigine, Letermovir, Licofelone, Lorazepam, Losartan, Lovastatin, Lumiracoxib, Midazolam, Mitiglinide, Morphine; Morphine Sulfate, Muraglitazar, Mycophenolate mofetil, Mycophenolic acid, Naldemedine, Nalmefene, Naltrexone, Naproxen, Nateglinide, Oxazepam, Paricalcitol, Pibrentasvir, Pitavastatin, Propofol, Raltegravir, Regorafenib, Rifampicin, Rucaparib, Seratrodast, Silodosin, Simvastatin, Sorafenib, Sulfamethoxazole, Suprofen, Tamoxifen, Tapentadol, Testosterone propionate, Topiroxostat, Trifluoperazine, Troglitazone, Vadimezan, Valdecoxib, Valproic Acid, Zaltoprofen, Zidovudine or Zileuton. In some embodiments, any of the disclosed drugs may be targeted by the modulating component of the system of the present disclosure. In some embodiments, the systems of the present disclosure is applicable in modulating the metabolism of at least one topoisomerase I inhibitor. In yet some further embodiments, the present disclosure encompasses modulatory component applicable in modulating any topoisomerase I inhibitor, for example, the CPT-11, also denoted herein as (4S)-4,11-Diethyl-3,4,12,14-tetrahydro-4-hydroxy-3,14-dioxo-1H-pyrano[3′,4′: 6,7]indolizino[1,2-b]quinolin-9-yl [1,4′-bipiperidine]-1′-carboxylic acid ester hydrochloride. CPT-11 or Irinotecan, sold under the brand name Camptosar among others, is a medication used to treat colon cancer, and small cell lung cancer. For colon cancer it is used either alone or with fluorouracil. For small cell lung cancer it is used with cisplatin. It is given by slow injection into a vein. Its IUPAC name is S)-4,11-diethyl-3,4,12,14-tetrahydro-4-hydroxy-3,14-dioxo1H-pyrano[3′,4′: 6,7]-indolizino[1,2-b]quinolin-9-yl-[1,4′bipiperidine]-1′-carboxylate and its Unique Ingredient Identifier (UNII) is 7673326042 (CAS number 97682-44-5). Irinotecan is further disclosed by Formula I:

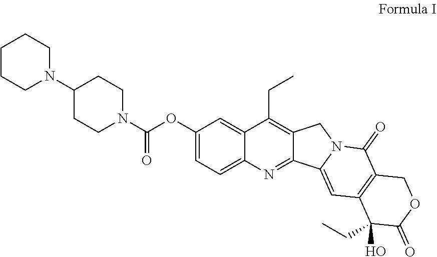 [SMILES: CCc1c2c(nc3ccc(OC(=O)N4CCC(N5CCCCC5)CC4)cc13)-c1cc3c(c(=O)n1C2)COC(=O)[C@]3(O)CC]

Still further, in some embodiments, the drug metabolism modulating component of the system disclosed herein inhibits or reduces the re-activation and/or conversion of the non-active metabolite of said CPT-11, SN38glucuronide (SN38G) to the active metabolite of the CPT-11, SN38 by bacterial GUS.

In yet some further embodiments, the system provided herein may comprise as a drug metabolism modulating component any nucleic acid sequence encoding an enzyme that modify target drugs. For example, any enzyme that activate, or alternatively inactivates a target drug or otherwise modulate the activity, bioavailability, clearance, stability, toxicity, and absorption of at least one drug.

In some non-limiting embodiments, the system may improve the pharmacokinetics of a drug by taking the prodrug approach. This approach may be utilized to convert the prodrug in the gut to the active parent drug and thus overcome ADME (absorption, distribution, metabolism and excretion) challenges, such as low oral absorption, poor stability, inadequate site specificity etc. In this system, the drug metabolism modulating component of the system promotes the activation of the prodrug to liberate the free parent drug in a timely and site-specific manner to increase the usefulness of the drug. The modulation is achieved by introducing to the targeted target cell, for example, the targeted bacteria, that in some embodiment, resides in the microbiome and is part of the microbiome population, a nucleic acid sequence that encodes at least one enzyme/s that catalyzes the conversion of such prodrug to a drug, or the conversion to an active or improved or potent metabolite. This enzyme/s may be produced and secreted by the bacteria to the gut lumen. There, in the gut, the introduced enzyme can recognize a specific linker present on the prodrug, digest it and free the active drug.

The components of the systems and methods of the present disclosure comprise nucleic acid sequence encoding, or alternatively being the modulating component or the selective component. The term ‘polynucleotide’ or a ‘nucleic acid sequence’ or ‘nucleic acid molecule’ refer herein to a polymer of nucleic acids, such as deoxyribonucleic acid (DNA) or ribonucleic acid (RNA). As used herein, ‘nucleic acid/s’ (also or nucleic acid molecule or nucleotide) refers to any DNA or RNA polynucleotides, oligonucleotides, fragments generated by the polymerase chain reaction (PCR) and fragments generated by any of ligation, scission, endonuclease action, and exonuclease action, either single- or double-stranded. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase).

As indicated herein above, in certain embodiments, the drug metabolism modulating component of the invention may comprise at least one CRISPR spacer that targets at least one nucleic acid sequence comprised within the selective component (that may be in some embodiment, a bacteriophage encoding toxin that comprise the protospacer, or essential genes of a lytic bacteriophage). In yet some further embodiments, the CRISPR array may also act as a modulating component. For example, the CRISPR array may further comprise in addition to the spacers that target the protospacer in the selective component, also at least one CRISPR spacer that targets a nucleic acid sequence comprised within said at least one gene encoding a protein involved in the metabolism of the drug (for example, the GusA protein). In such way the drug metabolism modulating component of the invention may target and/or inactivate both, the selective component (e.g., lytic phage, or other delivery vehicle or genetic element comprising a toxic element that inhibit or reduce cell viability and/or function), and the element participating in metabolism of the drug of interest. In yet some alternative embodiments, the modulating component may comprise at least one spacer suitable for one type of Cas nuclease that targets at least one protospacer comprised within the selective component, and at least one other spacer that is suitable for another type of Cas protein, that display reduced or abolished nuclease activity, fused to a further active domain that may in some embodiments recruit transcription activators that lead to increased transcription of the element that participates in metabolism of the drug, or any other nucleic acid modifying moiety that modify the target nucleic acid sequence, thereby modulating the element that participate in drug metabolism. Such modifying moiety may be any one of methyltransferase, a methylated DNA binding factor, a transcription factor, a transcription repressor, a chromatin remodeling factor, a polymerase, a demethylase, an acetylase, a deacetylase, a kinase, a phosphatase, an integrase, a recombinase, a ligase, a topoisomerase, a gyrase, a helicase, and any combinations thereof.

The modulatory component of the disclosed systems, compositions, kits and methods utilize the CRISPR-Cas system. As used herein, CRISPR arrays also known as SPIDRs (Spacer Interspersed Direct Repeats) constitute a family of DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein.

As used herein, the phrase “CRISPR array polynucleotide” refers to a DNA or RNA segment which comprises sufficient CRISPR repeats such that it is capable of down regulating (e.g. eliminating, targeting) a complementary gene.

According to one embodiment, the CRISPR array polynucleotide comprised within the modulating component of the system of the present disclosure, comprises at least 2 repeats with 1 spacer between them. In yet some further embodiments, the CRISPR array of the drug metabolism modulating component of the invention may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200 or more spacers. It should be further understood that the spacers of the drug metabolism modulating component of the invention may be either identical or different spacers.

In more embodiments, these spacers may target either an identical or different target protospacer/s and/or genes encoding a protein involved in metabolism of a specific drug. In yet some other embodiments, such spacer may target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more undesired gene/s.

In an exemplary embodiment, the CRISPR array polynucleotide comprises all of the CRISPR repeats, starting with the first nucleotide of the first CRISPR repeat and ending with the last nucleotide of the last (terminal) repeat.

As used herein, the term “spacer” refers to a non-repetitive spacer sequence that is found between multiple short direct repeats (i.e., CRISPR repeats) of CRISPR arrays. In some embodiments, CRISPR spacers are located in between two identical CRISPR repeats.

In some embodiments, CRISPR spacer is naturally present in between two identical, short direct repeats that are palindromic. It should be noted that the spacers of the invention may be located or present between two identical or not identical repeats, and moreover, these spacers encode crRNA that targets the proto-spacer within the pathogenic or undesired bacterial genes and/or proto-spacers within the selective component.

As used herein, the term “cas gene” refers to the genes that are generally coupled, associated or close to or in the vicinity of flanking CRISPR arrays that encode Cas proteins.

CRISPR arrays are typically found in the vicinity of four genes named cas1 to cas4. The most common arrangement of these genes is cas3-cas4-cas 1-cas2. The Cas3 protein appears to be a helicase, whereas Cas4 resembles the RecB family of exonucleases and contains a cysteine-rich motif, suggestive of DNA binding. The cas1 gene (NCBI COGs database code: COG1518) is especially noteworthy, as it serves as a universal marker of the CRISPR system (linked to all CRISPR systems except for that of Pyrococcus abyssii). cas2 remains to be characterized. cas1-4 are typically characterized by their close proximity to the CRISPR loci and their broad distribution across bacterial and archaeal species. Although not all cas1-4 genes associate with all CRISPR loci, they are all found in multiple subtypes.

Still further, three major types of CRISPR-Cas system are delineated: Type I, Type II and Type III. It should be appreciated that the nucleic acid of interest packaged within the modified bacteriophage of the invention may comprise CRISPR systems (e.g., gene encoding cas proteins and spacers) derived from any type of CRISPR-Cas system.

More specifically, Type I CRISPR-Cas systems contain the cas3 gene, which encodes a large protein with separate helicase and DNase activities, in addition to genes encoding proteins that probably form Cascade-like complexes with different compositions. These complexes contain numerous proteins that have been included in the repeat-associated mysterious proteins (RAMPs), which form a large superfamily of Cas proteins, and contain at least one RNA recognition motif (RRM; also known as a ferredoxin-fold domain) and a characteristic glycine-rich loop. RAMP superfamily encompasses the large Cas5 and Cas6 families on the basis of extensive sequence and structure comparisons. Furthermore, the Cas7 (COG1857) proteins represent another distinct, large family within the RAMP superfamily.

The type I CRISPR-Cas systems seem to target DNA where the target cleavage is catalyzed by the HD nuclease domains of Cas3. As the RecB nuclease domain of Cas4 is fused to Cas1 in several type I CRISPR-Cas systems, Cas4 could potentially play a part in spacer acquisition instead. It should be noted that any type I CRISPR-Cas systems may be applicable in the present invention, specifically, any one of type I-A, B, C, D, E, and F.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity, it is likely to be responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. This cleavage is catalyzed by the housekeeping, double-stranded RNA-specific RNase III in the presence of Cas9. Still further, type II system comprise at least one of cas9, cas1, cas2 csn2, and cas4 genes. It should be appreciated that any type II CRISPR-Cas systems may be applicable in the present invention, specifically, any one of type II-A or B.

The type III CRISPR-Cas systems contain polymerase and RAMP modules in which at least some of the RAMPs seem to be involved in the processing of the spacer-repeat transcripts, analogous to the Cascade complex. Type III systems can be further divided into sub-types III-A (also known as Mtube or CASS6) and III-B (also known as the polymerase-RAMP module). Subtype III-A systems can target plasmids, as has been demonstrated in vivo for S. epidermidis, and it seems plausible that the HD domain of the polymerase-like protein encoded in this subtype (COG1353) might be involved in the cleavage of target DNA. There is strong evidence that, at least in vitro, the type III-B CRISPR-Cas systems can target RNA, as shown for a subtype III-B system from furiosus. It should be appreciated that any cas gene that belongs to the type III CRISPR system may be used for the purpose of the invention, for example, any one of cas6, cas10, csm2, csm3, csm4, csm5, csm6, cmr1, cmr3, cmr4, cmr5, cmr6, cas1 and cas2. Still further, any one of typeIII-A or typeIII-B systems may be used for the systems, components and method of the invention. Of particular interest, specifically in cases where endogenous pathogenic or undesired genes are targeted by the systems and methods of the invention, the typeIII-B system may be used. In some particular embodiments, the at least one cas gene in the CRISPR-Cas system used as the nucleic acid sequence of interest in the vehicle of the invention, may be at least one cas gene of type I-E CRISPR system. The “type-IE CRISPR” system refers to native to K-type Escherichia coli. It has been shown to inhibit phage infection, cure plasmids, prevent conjugal element transfer and kill cells. This CRISPR machinery can be used to degrade specific intracellular DNA in an inducible and targeted manner, leaving the remainder DNA intact.

Still further, Cas proteins of the type V system my be also applicable in the modulating components disclosed herein. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF).

It should be noted that any CRISPR/Cas proteins may be used by the present disclosure, such Cas protein may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R. Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1. Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803. Elusimicrobium minutum Pei191, uncultured Termite group 1 bacterium phylotype Rs D17. Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657. Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K. Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1. Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345). Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

It should be appreciated that the present disclosure further encompasses in further aspects thereof, any of the components disclosed herein, and any nucleic acid sequence encoding the disclosed component and any construct, cassette or vector thereof. More specifically, in some embodiments, the present disclosure encompasses any of the drug metabolism modulating components disclosed herein, specifically, at least one component that may be in some embodiments, a nucleic acid molecule comprising at least one nucleic acid sequence encoding or modulating at least one element participating in metabolism of at least one drug, at least one cas gene and at least one CRISPR array. In yet some further embodiments, the present disclosure further comprises any construct, plasmid or delivery vehicle comprising the disclosed modulatory component. In yet some further embodiments, the present disclosure further encompasses any phage-based transducing particles, or bacteriophage-based vectors (e.g., bacteriophages that are capable of transducing the modulating component into a target cell) comprising the modulatory components disclosed herein. In yet some further embodiments, the present disclosure further encompasses any of the selective components as disclosed in the present disclosure. Specifically, in some embodiments, the selective component that comprises at least one protospacer that is recognized by the CRISPR array of the modulatory component. The selective component further comprises and/or encodes at least one toxic element that kill bacterial cells or disrupt, attenuate, and/or inhibit bacterial growth and/or function. In some embodiments, the present disclosure further provides nucleic acid molecules encoding the discussed selective component as well as any construct, plasmid, cassette, vector or vehicle comprising such selective component. In yet some further embodiments, the present disclosure further encompasses any phage-based transducing particles, or bacteriophage-based vectors (e.g., bacteriophages that are capable of transducing the modulating component into a target cell) comprising the disclosed selective component.

A further aspect of the present disclosure relates to a method for modulating drug metabolism of a bacterial cell/s and/or bacterial cell population. In some embodiments, the method comprising at least one of the following steps:

One step (a), that in some embodiment may be a first step, involves contacting the cell/s or any cell population comprising the cell/s with at least one drug metabolism modulating component comprising at least one nucleic acid sequence encoding or modulating at least one element participating in metabolism of at least one drug, at least one cas gene and at least one CRISPR array. Another step (b), that may be in some embodiments a second step, involves contacting the cells or any cell population comprising the cell/s with at least one selective component comprising at least one protospacer. It should be understood that the at least one protospacer is targeted by at least one spacer of the CRISPR array of (a) to inactivate the selective component.

Exposing and/or contacting the target cell and/or cell population to the drug metabolism modulating component and/or to the selective component, and/or to any system or composition comprising at least one of (a) and (b) components, leads to modulation of the drug metabolism of the target bacterial cell, and moreover, obtaining, selecting and/or enriching a population of bacterial cells exhibiting modified metabolism of at least one drug.

In some embodiments, “contacting” of the target cells with the systems and/or any components thereof, specifically, the modulatory component and/or the selective component as disclosed herein, or any delivery vehicle comprising these components, refers to exposing a cell or an environment in which the cell is located to one or more of the systems and/or any components thereof. Still further, “contacting” refers to the positioning of the systems and/or any components thereof, specifically, the modulatory component and/or the selective component as disclosed herein, or any delivery vehicle comprising these components (e.g., bacteriophages) such that they are in direct or indirect contact with the target cells (e.g., bacterial cells). It should be understood that in some embodiments, the term “contacting” includes the in vivo as well as the in vitro exposure of cells to the components or compositions disclosed herein. Thus, in some embodiments, “contacting” includes administering the at least one system and/or any components thereof, specifically, the modulatory component and/or the selective component as disclosed herein, or any delivery vehicle comprising these components, or any cells or population of cells (e.g., bacterial cells) or any composition thereof, to an individual.

In some embodiments, the at least one nucleic acid sequence encoding or modulating at least one element participating in metabolism of the drug is comprised within the CRISPR array.

In some embodiments, the step of contacting the target cells with the systems of the present disclosure or with any components thereof, as provided by the present disclosure, may in some embodiments involve transformation and/or transduction of the nucleic acid sequences of the components of the system of the present disclosure to the target cell, for example, the target bacterial cell. “Transformation” and “transfection” means the introduction of a nucleic acid, e.g., naked DNA or the delivery vehicle as herein defined, into a recipient cell/s by nucleic acid-mediated gene transfer.

“Transduction” refers to a process by which foreign DNA is introduced into a cell by a virus or viral vector. An example is the viral transfer of DNA from one bacterium to another and hence an example of horizontal gene transfer.

In yet some further embodiments, the modulating component used by the methods disclosed herein, is comprised within at least one delivery vehicle that specifically targets the cell/s and/or the cells in a bacterial cell population.

In yet some further embodiments, the selective component used by the methods disclosed herein, is comprised within at least one delivery vehicle that specifically targets the cell/s and/or the cells in a bacterial cell population. In some embodiments, the selective component comprises at least one agent that affects cell viability and/or activity.

In yet some further embodiments, the at least one delivery vehicle that comprises the selective component used by the methods disclosed herein, is or comprises at least one genetic element. More specifically, in some embodiments, such genetic element is at least one of: at least one transducing particle, at least one bacteriophage or any fragments or parts thereof, at least one bacteriophage-based or bacteriophage-like transducing particle and/or at least one modified bacteriophage, at least one vector, plasmid and/or construct, that comprises and/or encodes at least one of the modulating component and/or selective component and any combinations or cocktails thereof.

In some embodiments, the delivery vehicle of the selective component of the disclosed system may be a bacteriophage-based particle that comprises endogenous or exogenous toxic element that kill bacterial cells or disrupt, attenuate, and/or inhibit bacterial growth.

In some embodiments, the delivery vehicle of the selective component, may be a bacteriophage, or bacteriophage-based transducing particle that endogenously comprise a toxic element that that kill bacterial cells or disrupt, attenuate, and/or inhibit bacterial growth. In some embodiments, such bacteriophage or bacteriophage-based transducing particles may be used as the selective component of the disclosed system. In some embodiments, a bacteriophage or bacteriophage-based transducing particle used as the selective component or used to deliver the selective component, may be a lytic bacteriophage. In some embodiments such lytic bacteriophage may be either modified, recombinant, or non-modified bacteriophage, or alternatively, of lytic-phage-based particle.

In yet some further embodiments, the delivery vehicle of the selective component may be a plasmid, construct, or any other genetic element that comprises a nucleic acid sequence encoding the toxic element that kill bacterial cells or disrupt, attenuate, and/or inhibit bacterial growth and/or function.

In yet some further embodiments, the delivery vehicle that comprises at least one of the components of the systems used by the methods disclosed herein is at least one bacteriophage or any bacteriophage-like transducing particle or bacteriophage-based transducing particle that comprises and/or encodes at least one toxic element that kill bacterial cells or disrupt, attenuate, and/or inhibit bacterial growth and/or function.

In some embodiments, the bacteriophage or bacteriophage-like transducing particle is of the T7-like viruses, the T4-like viruses or the Escherichia virus Lambda.

In some embodiments, the target cell/s targeted by the disclosed system is a bacterial cell. In yet some further embodiments, the bacterial cell/s and/or cell population targeted by the methods disclosed herein is or comprised within gut microbiome cell population. Still further, in some embodiments, the cell population comprises at least one bacteria of the Proteobacteria Firmicutes, Bacteroidetes, Actinobacteria, and phyla, or any mutant, variant of isolate or any combination thereof.

In some embodiments, the drug metabolism modulating component used by the methods disclosed herein, affects at least one of the activity, bioavailability, clearance, stability, toxicity, and absorption of the drug.

In yet some further embodiments, the element that participates in the metabolism of the at least one drug targeted by the modulating component used by the methods disclosed herein is at least one glucuronide enzyme.

In more specific embodiments glucuronide enzyme is at least one β-glucuronidase (GUS) enzyme. Still further, in some embodiments, the modulating component comprises nucleic acid sequence that encodes at least one element that repress the transcription of at least one β-glucuronidase (GUS) enzyme.

In some embodiments, the element encoded by the nucleic acid sequence of the modulating component represses the transcription of said BGUS enzyme. In yet some further embodiments, the element is a Gus Repressor (GusR).

In some embodiments, the GusR display reduced or abolished affinity to at least one GUS ligand and/or substrate. In some embodiments, the GusR used in the modulating component of the system of the disclosed methods, comprise at least one substitution and/or other modification in residues that form the ligand-binding pocket of the GusR. Thus, in yet some further embodiments, any residue/s that form and/or participate in the ligand-binding pocket of the GusR can be manipulated to obtain a GusR that display reduced or abolished affinity to at least one GUS ligand and/or substrate. Specifically, any GusR that comprises substation in at least one of H126, K125, R69, E97, R73, Y164, L160, T163 and M87, is applicable in the modulating component of the present disclosure.

Still further, in some embodiments, the GusR carry at least one mutation in at least one of: lysine (K) 125 to Alanine (A), Tyrosine (Y) 164 to Phenylalanine (F), and Arginine (R) 73 to Alanine (A), and any combinations thereof. It should be understood that the disclosed GusR mutants and any combinations thereof useful in the present disclosure are also applicable in any of the methods disclosed herein.

In yet some further embodiments, at least one drug modulated by the system used by the methods disclosed herein is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, cns depressant, anticholinergic agent, parasympatholytic agent, immunosuppressant, opioid antidote, anti-asthmatic agent, anti-benign prostatic hyperplasia or anti-gout agent.

In yet some further embodiments, the drug is at least one antineoplastic agent, optionally, at least one topoisomerase I inhibitor.

It should be understood that any of the drug metabolized by Gus enzymes as disclosed herein in connection with other aspects of the present disclosure are also applicable in this aspect as well. In more specific embodiments, the drug modulated by the methods disclosed herein is at least one topoisomerase I inhibitor. In yet some further embodiments, the topoisomerase I inhibitor is Camptothecin-11 (CPT-11, Irinotecan), or any derivatives, metabolites or biosimilars thereof. In some embodiments, this drug is also named (4S)-4,11-Diethyl-3,4,12,14-tetrahydro-4-hydroxy-3,14-dioxo-1H-pyrano[3′,4′: 6,7]indolizino[1,2-b]quinolin-9-yl [1,4′-bipiperidine]-1′-carboxylic acid ester hydrochloride.

In some embodiments, the drug metabolism modulating component used by the methods disclosed herein inhibits or reduces the re-activation and/or conversion of the non-active metabolite of the CPT-11, into an active metabolite, by GUS, specifically, bacterial GUS. More specifically, in some embodiments, the modulatory component of the present disclosure reduces or inhibits the enzymatic reaction by GUS, specifically, hydrolysis of the inactive metabolite of irinotecan, SN38G, into the active metabolite SN38.

In some embodiments, step (b) of the methods disclosed herein, specifically, contacting the cells and/or cell population with the selective component of the system disclosed herein, may be repeated one time or even several times. For example, 2, 3, 4, 5, 6., 7, 8, 9, 10, 15, 20 or more times. In some embodiments, this selective step is repeated at least twice to enrich said population of modified cells displaying modulated metabolism of at least one drug.

In some embodiments, the modulatory methods disclosed herein, may be performed in a subject in need. Accordingly, the contacting step as used herein may involve in some embodiments, administration of the components of the disclosed systems or any compositions and kits thereof, to the treated subject.

In some embodiments, such administration is performed prior to, together with, or subsequently to the administration of at least one drug.

A further aspect of the present disclosure relates to at least one cell and/or a population of said cells, or any composition or product thereof. In some embodiments, the cells are bacterial cells or population of cells comprising said bacterial cells. The disclosed cells or population of cells comprising the cell/s are characterized by, and/or exhibiting modified metabolism of at least one drug. These cells were contacted with the at least one component/s (the modulatory component, the selective component or both) of the systems disclosed herein, and therefore display modified metabolism of at least one drug. Modified metabolism of the drug is meant any change in the metabolism of the drug by the same, for example, causing or alternatively, preventing any enzymatic or chemical modification/s of the drug that affect/s stability, activity, and/or bioavailability thereof. In some embodiments, the cell and/or cell population is prepared by a method comprising at least one of the following steps. In one step (a), that may be in some embodiments a first step, the method involves contacting the cell/s or any cell population comprising the target cell/s with at least one drug metabolism modulating component comprising at least one nucleic acid sequence encoding or modulating at least one element participating in metabolism of at least one drug, at least one cas gene and at least one CRISPR array. In another step (b), that may be in some embodiments the next step or the second step (b), contacting the cells or any cell population comprising the cell/s with at least one selective component comprising at least one protospacer. It should be understood that at least one protospacer is targeted by at least one spacer of the CRISPR array of (a), to inactivate said selective component.

In some embodiment, the cell and/or cell population disclosed herein is a bacterial cell and/or cell population. Still further, in some embodiments, the bacterial cell and/or a population comprising the cell provided by the present invention, is prepared by a method as disclosed by the present disclosure.

In yet some further embodiments, the modulating component used in the method for the preparation of the cell and/or a population disclosed herein (e.g., bacterial cell/s), comprises nucleic acid sequence that encodes at least one element that repress inhibits and/or reduces the expression and/or activity and/or stability of at least one β-glucuronidase (GUS) enzyme.

In yet some further embodiments, the element encoded by the nucleic acid sequence of the modulating component used in the method for the preparation of the cell and/or a cell population ((e.g., bacterial cell/s) disclosed herein, may be an element that represses the transcription of said βGUS enzyme. In yet some further optional embodiments, the element may be a Gus repressor (GusR).

Still further, in some embodiments of the disclosed cell and/or a population comprising such cell/s, the GusR display reduced or abolished affinity to at least one GUS ligand and/or substrate. In yet some further optional embodiments, such GusR carry at least one mutation in at least one of: lysine (K) 125, Tyrosine (Y) 164 and/or Arginine (R) 73. More specifically, the GusR mutant encoded by the nucleic acid sequence of the modulating component may be at least one GusR that carry at least one substitution of lysine (K) 125 to Alanine (A), Tyrosine (Y) 164 to Phenylalanine (F), and Arginine (R) 73 to Alanine (A), and any combinations thereof. In some specific and non-limiting embodiments, the GusR mutant encoded by the nucleic acid sequence of the modulating component used in the preparation of the disclosed cells or cell populations may comprise the amino acid sequence of any one of SEQ ID NO:9, 11, 13, respectively.

In some embodiments, the cell and/or cell population (e.g., bacterial cell/s) disclosed herein display modified metabolism of at least one drug, that may be at least one antineoplastic agent. In some optional embodiments, the drug is at least one topoisomerase I inhibitor.

Still further, in some embodiments, the cell and/or cell population (e.g., bacterial cell/s) disclosed herein, display modulated metabolism of Camptothecin-11 (CPT-11, Irinotecan), or any derivatives thereof.

In yet some further embodiments, the cell and/or cell population display inhibited or reduced re-activation and/or conversion of the non-active metabolite of CPT-11, the SN38G, to the active metabolite of said CPT-11, SN38, by GUS. For example, by bacterial GUS.

A further aspect of the present disclosure relates to a composition and/or kit, specifically, a pharmaceutical composition, a combined composition and/or kit comprising at least one drug and at least one of the systems of the invention that lead to modulated metabolism of the drug of the disclosed composition, or any cell or cell population that display modulated metabolism of the drug. In more specific embodiments, the composition or kit disclosed herein may comprise:
(I) at least one drug; and at least one of:
(II) at least one drug metabolism modulatory system comprising at least one of: (a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and (b) at least one selective component comprising at least one protospacer. It should be noted that wherein said at least one protospacer is targeted by at least one spacer of the CRISPR array of (a), to inactivate said selective component. In some alternative or additional embodiments, the composition may further comprise, or alternatively comprise in addition to the drug, also (III), at least one bacterial cell and/or a population of the bacterial cells, or any composition or product thereof, exhibiting modified metabolism of the at least one drug. Still further, in some embodiments the composition optionally further comprises at least one of pharmaceutically acceptable carrier/s, diluent/s, excipient/s and additive/s.

Still further, in case of a kit as disclosed herein, according to some embodiments, each of the elements of the kit, specifically, elements (I), (II), (III) may be provided in a first, second and/or third dosage forms. In yet some further embodiments, the kit may further comprise a container comprising the various elements of the kit, e.g., the various dosage forms.

In some embodiments, the system of the composition disclosed herein may be any of the systems defined by the present disclosure. Still further, the cell or cell population of the composition disclosed herein may be as defined by the present disclosure.

In yet some further embodiments, the at least one drug comprised within and targeted by the composition or kit disclosed herein, is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, CNS depressant, anticholinergic agent, parasympatholytic agent, immunosuppressant, opioid antidote, anti-asthmatic agent, anti-benign prostatic hyperplasia or anti-gout agent.

In yet some further embodiments, the drug is at least one antineoplastic agent. In some optional embodiments, the drug may be at least one topoisomerase I inhibitor.

In yet some particular and non-limiting embodiments, the drug is Camptothecin-11 (CPT-11, Irinotecan), or any derivatives thereof.

Still further, it should be understood that any of the drugs disclosed in the present disclosure in connection with other aspects of the invention, is also applicable in the present aspect as well. In yet some alternative embodiments, the composition of the present disclosure, as well as the disclosed systems and any components thereof, specifically, the modulatory component and/or the selective component as disclosed herein, or any cells or population of cells (e.g., bacterial cells) comprising the same, may be formulated as an injectable dosage form. In yet some further embodiments, the composition disclosed herein may be formulated in an injectable dosage unit form.

In some embodiments, the oral dosage form may be administered orally, for example, as a solution (e.g., syrup), or as a powder, tablet, capsule, and the like. In certain embodiments the composition of the invention may be formulated in a formulation adapted for add-on to a solid, semi-solid or liquid food, beverage, food additive, food supplement, medical food, botanical drug, drug and/or any type of pharmaceutical compound.

In some embodiments, the add-on composition according to the present disclosure that comprises the disclosed systems and/or any components thereof, specifically, the modulatory component and/or the selective component as disclosed herein, or any delivery vehicle comprising these components, or any cells or population of cells (e.g., bacterial cells) comprising the same, may be formulated as a food additive, food supplement or medical food. In other embodiment, such add-on composition of the invention may be further added or combined with drugs or any type of pharmaceutical products. The term ‘add-on’ as used herein is meant a composition or dosage unit form of the at least one systems and/or any components thereof, specifically, the modulatory component and/or the selective component as disclosed herein, or any delivery vehicle comprising these components, or any cells or population of cells (e.g., bacterial cells) comprising the same of the present disclosure that may be added to existing compound, composition or material (e.g., food or beverage), enhancing or modulating desired properties thereof or alternatively, adding specific desired property to an existing compound, composition, food or beverage.

More specifically, in certain embodiments, the at least one system and/or any components thereof, specifically, the modulatory component and/or the selective component as disclosed herein, or any delivery vehicle comprising these components, or any cells or population of cells (e.g., bacterial cells) of the present disclosure, or any dosage form or composition thereof may be an add-on to a food supplement, or alternatively, may be used as a food supplement. A food supplement, the term coined by the European Commission for Food and Feed Safety, or a dietary supplement, an analogous term adopted by the FDA, relates to any kind of substances, natural or synthetic, with a nutritional or physiological effect whose purpose is to supplement normal or restricted diet. In this sense, this term also encompasses food additives and dietary ingredients. Further, under the Dietary Supplement Health and Education Act of 1994 (DSHEA), a statute of US Federal legislation, the term dietary supplement is defined as a product intended to supplement the diet that bears or contains one or more of the following dietary ingredients: a vitamin, a mineral, an herb or other botanical, a dietary substance for use by a subject to supplement the diet by increasing the total dietary intake, or a concentrate, metabolite, constituent, extract, or combination of any of the aforementioned ingredients Food or dietary supplements are marketed a form of pills, capsules, powders, drinks, and energy bars and other dose forms. Unlike drugs, however, they are mainly unregulated, i.e., marketed without proof of effectiveness or safety. Therefore, the European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet. However, the add-on dosage form or composition that comprise the systems and/or any components thereof, specifically, the modulatory component and/or the selective component as disclosed herein, or any delivery vehicle comprising these components, or any cells or population of cells (e.g., bacterial cells) provided herein, may be added to a meal or beverage consumed by the subject.

In yet some further embodiments, the systems and/or any components thereof, specifically, the modulatory component and/or the selective component as disclosed herein, or any delivery vehicle comprising these components, or any cells or population of cells (e.g., bacterial cells) or any composition thereof, in accordance with the present disclosure may be an add-on to medical foods or may be consumed as a medical food. Further in this connection should be mentioned medical foods, which are foods that are specially formulated and intended for the dietary management of a disease that has distinctive nutritional needs that cannot be met by normal diet alone. Of particular interest are add-on formulations of the disclosed components, delivery vehicles comprising the disclosed components (the modulatory and/or the selective components) systems and/or cells, that may be formulated as add-o formulations to a drug, specifically, any of the drugs disclosed by the present disclosure (e.g., in Table 1). More specifically, as a add-on formulation to Irinotecan, or any derivatives and biosimilars thereof.

More specifically, pharmaceutical compositions according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the nucleic acid delivery vehicle/s of the present disclosure (e.g., modulatory component and/or the selective component of the disclosed system) with liquid carriers or finely divided solid carriers or both, and then, if necessary, shaping the product. The compositions may be formulated into any of many possible dosage forms such as, but not limited to, tablets, pills, capsules, liquid syrups, soft gels, sprays, matrixes, suppositories and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. The active agent may be systemic after administration or may be localized by the use of regional administration, intramural administration, or use of an implant that acts to retain the active dose at the site of implantation. The active agent (e.g., the modulatory and/or selective components and systems thereof, may be formulated for immediate activity or it may be formulated for sustained release.

It should be appreciated that the composition of the disclosed systems and/or any components thereof, specifically, the modulatory component and/or the selective component as disclosed herein, or any delivery vehicle comprising these components, or any cells or population of cells (e.g., bacterial cells), may be formulated in any appropriate formulation.

As used interchangeably herein, “dosage units”, “dosage forms”, “oral or injectable dosage units”, “dosage unit forms”, “oral or injectable dosage unit forms” and the like refer to both, solid dosage forms as known in the art, or to a liquid dosage form. The dosage forms are intended for peroral use, i.e., to be swallowed (ingested), or even injected or applicated in any other means, either by a subject in need thereof, or for administration by a medical practitioner. The terms “active substance” or “active ingredient”, used herein interchangeably, refer to a therapeutically or physiologically active substance, specifically, the systems and/or any components thereof, specifically, the modulatory component and/or the selective component as disclosed herein, or any delivery vehicle comprising these components, or any cells or population of cells (e.g., bacterial cells) disclosed herein, that provides a therapeutic/physiological effect to a patient, and can also refer to a mixture of at least two thereof.

As indicated herein, the composition or any dosage form or dosage unit form disclosed herein may be provided in an injectable formulation. The term “injection” or “injectable” as used herein refers to a bolus injection (administration of a discrete amount of the at least one systems and/or any components thereof, specifically, the modulatory component and/or the selective component as disclosed herein, or any delivery vehicle comprising these components, or any cells or population of cells (e.g., bacterial cells) disclosed herein, for raising its concentration in a bodily fluid), slow bolus injection over several minutes, or prolonged infusion, or several consecutive injections/infusions that are given at spaced apart intervals. Such spaced apart injections per a single administration are also referred to herein as “per administration injection”, or in other words, a single administration can include several injections or prolonged infusion. The injectable aqueous formulation for non-systemic administration to a subject in need thereof as herein defined may be administered using a drug-device combination, for example a mechanical or electro-mechanical device, more preferably an electro-mechanical infusion pump. The electro-mechanical pump, for example, consists of a reservoir for housing a medication, a catheter having a proximal portion coupled to the pump and having a distal portion adapted for administering a medication to the desired site.

A further aspect of the present disclosure relates to a method for modulating the metabolism of at least one drug in a subject in need thereof. More specifically, the method comprising the step of administering to the subject an effective amount of at least one of, the system disclosed herein, the modified bacterial cell/s or cell populations, any compositions or kits thereof, or any combinations thereof with at least one drug. More specifically, in some embodiments, the treated subject is administered with at least one of: (I), at least one drug metabolism modulatory system comprising at least one of: (a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and (b) at least one selective component comprising at least one protospacer. The at least one protospacer is targeted by at least one spacer of the CRISPR array of (a), to inactivate said selective component. Alternatively, or additionally, the treated subject is administered with (II), at least one bacterial cell and/or a population of the bacterial cells, or any composition or product thereof, exhibiting modified metabolism of the at least one drug. Alternatively, or additionally, the treated subject is administered with (III), at least one composition, kit or system comprising at least one of (I) and (II). Still further, in some embodiments, the subject may be administered with (IV), any combination of the at least one of (I), (II) and (III) with the at least one drug.

In some embodiments the target cell/s targeted by the systems used by the methods disclosed herein, are part of at least one microbiome of the treated subject. Thus, the disclosed methods target cells in the microbiome of the treated subject, thereby leading to modulation of the metabolism of the specific drug by the microbiome cells.

In some embodiments, the methods disclosed herein may use any of the system/s as defined by the present disclosure, the cell or cell population used by the method is as defined herein, and the composition is as defined by the present disclosure.

In some embodiments, the methods, systems and compositions disclosed herein target bacteria that are part of at least one microbiome, specifically, of the treated subject. In some embodiments, the bacteria are of the gut microbiome. However, it should be appreciated that any bacteria of any microbiome of the subject may be targeted by the systems, compositions and methods disclosed herein.

In some embodiments, where the system is introduced to the subject, the modulation of the metabolism of the disclosed drug/s is performed in vivo. In some embodiments, to obtain an enriched microbiome population of cells exhibiting modified metabolism of at least one drug, the selective step may be repeated at least twice. Specifically, the administration of the selective component to the treated subject may be performed 2, 3, 4, 5, 6, 7, 8, 9, 10, 15, 20, 30, 40, 50, 60, 70, 80, 90, 00 times or more, as required. In yet some further embodiments, the administration of both components, the modulating component (a), and the selective component (b), may be performed twice or more times to the treated subject.

In some embodiments, the modulation is performed ex vivo in cells or cell population obtained from the subject or a donor subject and an effective amount of at least one microbiome population of bacterial cells exhibiting, or characterized by, modified metabolism of the at least one drug is administered to the subject.

Still further, in a further aspect, the invention provides a method for treating, preventing, ameliorating, reducing or delaying the onset of at least one pathologic disorder in a subject in need thereof. More specifically, the method comprising the step of administering to the subject an effective amount of at least one of, at least one drug, the system disclosed herein, the modified bacterial cell/s or cell populations, any compositions or kits thereof, or any combinations thereof with at least one drug. More specifically, in some embodiments, the treated subject is administered with at least one of: (I), at least one drug metabolism modulatory system comprising at least one of (a) at least one drug metabolism modulating component comprising at least one nucleic acid sequence encoding or modulating at least one element participating in metabolism of at least one drug, at least one cas gene and at least one CRISPR array; and (b) at least one selective component comprising at least one protospacer The at least one protospacer is targeted by at least one spacer of the CRISPR array of (a), to inactivate the selective component. Alternatively, or additionally, the treated subject is administered with (II), at least one bacterial cell and/or a population of the bacterial cells, or any composition or product thereof, exhibiting modified metabolism of the at least one drug. Alternatively, or additionally, the treated subject is administered with (III), at least one composition, kit or system comprising at least one of (I) and (II); and (IV) any combination of said at least one of (I), (II) and (III) with the at least one drug.

In some embodiments, the therapeutic methods disclosed herein may use any of the system/s as defined by the present disclosure, the cell or cell population used by the method is as defined herein, and the composition is as defined by the present disclosure.

In some embodiments, the methods disclosed herein may be applicable for treating any pathologic disorder. In some embodiments, the disorder may be any disorder treated by a drug. In yet some further embodiments, such disorders may be at least one of a neoplastic disorder, a viral infection, an inflammatory disorder, an immune-cell mediated disorder, a metabolic disorder and an autoimmune disorder.

In yet some further embodiments of the therapeutic methods disclosed herein, the drug used and/or modulated by the methods is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, cns depressant, anticholinergic agent, parasympatholytic agent, immunosuppressant, opioid antidote, anti-asthmatic agent, anti-benign prostatic hyperplasia or anti-gout agent.

Still further, in some embodiments, the subject treated by the methods disclosed herein is a subject suffering of at least one neoplastic disorder.

In yet some further embodiments, the drug is at least one antineoplastic agent. In yet some optional embodiments, the drug used and modulated by the disclosed method may be at least one topoisomerase I inhibitor.

Still further, in some embodiments the drug used and/or modulated by the therapeutic methods of the invention is Camptothecin-11 (CPT-11, Irinotecan), or any derivatives thereof.

In some embodiments, the element encoded by the nucleic acid sequence of the modulating component used by the disclosed method may represses the transcription of the βGUS enzyme. In some optional embodiments, the element is a Gus Repressor (GusR).

In some embodiments, the GusR display reduced or abolished affinity to at least one GUS ligand and/or substrate.

In yet some further embodiments, the GusR carry at least one mutation in at least one of: lysine (K) 125, Tyrosine (Y) 164 and/or Arginine (R) 73. More specifically, the GusR mutant encoded by the nucleic acid sequence of the modulating component may be at least one GusR that carry at least one substitution of lysine (K) 125 to Alanine (A), Tyrosine (Y) 164 to Phenylalanine (F), and Arginine (R) 73 to Alanine (A), and any combinations thereof. In some specific and non-limiting embodiments, the GusR mutant encoded by the nucleic acid sequence of the modulating component used in the preparation of the disclosed cells or cell populations may comprise the amino acid sequence of any one of SEQ ID NO: 9, 11 and 13, respectively.

The present disclosure provides therapeutic methods for treating neoplastic disorders or cancer. The present disclosure may be applicable for any proliferative disorder that may be in some embodiments any neoplastic disease. More specifically, any abnormal mass of tissue, also referred to herein as tumor, that formed due to uncontrolled or abnormal cell growth that results in increased cell number. The methods of the present disclosure may be applicable in some embodiments for any neoplasm, either benign neoplasms, in situ neoplasms, or malignant neoplasms. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions and systems of the present invention may be applicable for a patient suffering from any one of non-solid and solid tumors. Malignancy, as contemplated in the present invention may be any one of carcinomas, melanomas, lymphomas, leukemia, myeloma and sarcomas. Therefore, in some embodiments any of the methods of the invention (specifically, therapeutic methods), systems and compositions disclosed herein, may be applicable for any of the malignancies disclosed by the present disclosure.

More specifically, carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas. Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

In some embodiments, the methods of the present disclosure may be applicable for any solid tumor. In more specific embodiments, the methods disclosed herein may be applicable for any malignancy that may affect any organ or tissue in any body cavity, for example, the peritoneal cavity (e.g., liposarcoma), the pleural cavity (e.g., mesothelioma, invading lung), any tumor in distinct organs, for example, the urinary bladder, ovary carcinomas, and tumors of the brain meninges. Particular and non-limiting embodiments of tumors applicable in the methods, compositions and system of the present disclosure may include but are not limited to at least one of colorectal carcinoma, small cell lung carcinoma, ovarian cancer, liver carcinoma, breast cancer, pancreatic cancer, brain tumors and any related conditions, as well as any metastatic condition, tissue or organ thereof. It should be understood that the methods, compositions, kits and systems of the present disclosure are applicable for any type and/or stage and/or grade of any of the malignant disorders discussed herein or any metastasis thereof. Still further, it must be appreciated that the methods, compositions and systems of the invention may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

Still further, in some embodiments, the methods, compositions, kits, and systems of the present disclosure are applicable for any type and/or stage and/or grade of any metastasis, metastatic cancer or status of any of the cancerous conditions disclosed herein.

As used herein the term “metastatic cancer” or “metastatic status” refers to a cancer that has spread from the place where it first started (primary cancer) to another place in the body. A tumor formed by metastatic cancer cells originated from primary tumors or other metastatic tumors, that spread using the blood and/or lymph systems, is referred to herein as a metastatic tumor or a metastasis. Further malignancies that may find utility in the present invention can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell ca-cinoma-see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer). Still further, in some embodiments, the methods, systems, compositions and kits of the present disclosure are applicable for colon carcinoma. In some specific embodiments, the present disclosure provides therapeutic methods for treating any of the disclosed pathologies, and specifically any pathology treated by the target drug. For example, topoisomerase I inhibitor, specifically, Irinotecan.

Colorectal cancer (CRC), also known as bowel cancer colon cancer, or rectal cancer, is the development of cancer from the colon or rectum. Signs and symptoms may include blood in the stool, a change in bowel movements, weight loss, and fatigue. Most colorectal cancers are due to old age and lifestyle factors, with only a small number of cases due to underlying genetic disorders. Risk factors include diet, obesity, smoking, and lack of physical activity. Thus, in some embodiments, the present disclosure specifically provides therapeutic methods for the treatment of Colorectal cancer.

In some embodiments, the methods, systems, compositions and kits of the present disclosure are applicable for small lung cell carcinoma. Small-cell carcinoma of the lung is also known as small-cell lung cancer (SCLC) or oat-cell cancer because the cancer cells may appear to look like oats under a microscope. Small-cell carcinoma is a type of cancer that can appear in various parts of the body, but most often occurs in the lung. It can grow very rapidly and spread to other organs. About 10-15 percent of lung cancers are small-cell carcinomas. Smoking tobacco is the most significant risk factor. There are multiple types of small-cell carcinoma. Combined small-cell carcinoma occurs alongside other types of lung cancer, such as squamous cell carcinoma or adenocarcinoma. in some embodiments, the present disclosure specifically provides therapeutic methods for the treatment of small-cell lung cancer (SCLC).

Still further, it must be appreciated that in some embodiments, the methods, systems, compositions and kits of the present disclosure are applicable for any of the disclosed disorders, as well as to any symptoms, conditions and complications thereof. Such symptoms may include for example, diarrhea (fecal staining of skin, lose watery stool) and bloody diarrhea (black sticky stool), loss or gain of body weight, and the like.

By “patient” or “subject in need” it is meant any organism who may be in need of at least one drug and to whom the system/s and methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the system/s and method/s of the invention are intended for mammals. It should be noted that specifically in cases of non-human subjects, the method of the invention may be performed using administration via injection, drinking water, feed, spraying, oral gavage and directly into the digestive tract of subjects in need thereof.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease. The term “treatment or prevention” as used herein, refers to the complete range of therapeutically positive effects of administrating to a subject including inhibition, reduction of, alleviation of, and relief from, pathologic disorder involved with at least one short term cellular stress condition/process and any associated condition, illness, symptoms, undesired side effects or related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the at least one short term cellular stress condition/process and their symptoms, slowing their progress, further exacerbation or development, so as to appear later than in the absence of the treatment according to the invention.

As indicated above, the methods and compositions provided by the present invention may be used for the treatment of a “pathological disorder”, i.e., pathologic disorder or condition involved with at least one short term cellular stress condition/process, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods, systems and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Materials

The following drugs shown to be metabolized by the human UGT enzymes and are potentially be metabolized and effected by the bacterial GUS enzymes. These drugs are used in the present disclosure (Example 6).

TABLE 1
Medications metabolized by the human UGT enzymes and
potentially affected by the microbiome GUS enzymes

| | | Route of | PubChem | Drug |
|---|---|---|---|---|
| Medication | Therapeutic Category | Administration | CID | Bank ID |
| Abacavir | Antiviral agent | Oral | 441300 | DB01048 |
| Acemetacin | Analgesic, antipyretic and anti- | Oral | 1981 | DB13783 |
| | inflammatory agent | | | |
| Acetaminophen | Analgesic, antipyretic and anti- | Oral, Rectal, | 1983 | DB00316 |
| | inflammatory agent | Intravenous | | |
| Ambrisentan | Antihypertensive agent | Oral | 6918493 | DB06403 |
| Artenimol | Antimalarial agent | Oral | 6918483 | DB11638 |
| Asenapine | Neuroleptic agent | Sublingual | 11954293 | DB06216 |
| Atorvastatin | Anticholesteremic agent | Oral | 60823 | DB01076 |
| Axitinib | Antineoplastic agent | Oral | 6450551 | DB06626 |
| Bictegravir | Antiviral agent | Oral | 90311989 | DB11799 |
| Buprenorphine | Analgesic, antipyretic and anti- | Buccal, | 644073 | DB00921 |
| | inflammatory agent | Sublingual, | | |
| | | Transdermal | | |
| Canagliflozin | Anti-diabetic agent | Oral | 24812758 | DB08907 |
| Candesartan | Antihypertensive agent | Oral | 2541 | DB13919 |
| Candesartan | Antihypertensive agent | Oral | 2540 | DB00796 |
| cilexetil | | | | |
| Carbamazepine | Antiepileptic agent | Oral | 2554 | DB00564 |
| Chenodeoxycholic | Bile acid | Oral | 10133 | DB06777 |
| acid | | | | |
| Clozapine | Neuroleptic agent | Oral | 2818 | DB00363 |
| Codeine | Analgesic, antipyretic and anti- | Oral, | 5284371 | DB00318 |
| | inflammatory agent | Intramuscular, | | |
| | | Subcutaneous | | |
| Cyproheptadine | Anti-asthmatic agent | Oral | 2913 | DB00434 |
| Dabigatran | Anticoagulant | Oral | 6445226 | DB06695 |
| etexilate | | | | |
| Dapagliflozin | Anti-diabetic agent | Oral | 9887712 | DB06292 |
| Deferiprone | Iron chelator | Oral | 2972 | DB08826 |
| Delafloxacin | Antibacterial agent | Oral, | 487101 | DB11943 |
| | | Intravenous | | |
| Diclofenac | Analgesic, antipyretic and anti- | Oral, Topical, | 3033 | DB00586 |
| | inflammatory agent | Ophthalmic | | |
| Diflunisal | Analgesic, antipyretic and anti- | Oral | 3059 | DB00861 |
| | inflammatory agent | | | |
| Dolutegravir | Antiviral agent | Oral | 54726191 | DB08930 |
| Eltrombopag | Hematopoietic growth factor | Oral | 9846180 | DB06210 |
| Enasidenib | Antineoplastic agent | Oral | 89683805 | DB13874 |
| Entacapone | Antiparkinsonism agent | Oral | 5281081 | DB00494 |
| Epirubicin | Antineoplastic agent | Intravenous | 41867 | DB00445 |
| Erlotinib | Antineoplastic agent | Oral | 176870 | DB00530 |
| Ertugliflozin | Anti-diabetic agent | Oral | 44814423 | DB11827 |
| Estradiol | Hormone | Oral, Vaginal, | 5757 | DB00783 |
| | | Transdermal | | |
| Estradiol acetate | Hormone | Vaginal | 9818306 | DB13952 |
| Estradiol | Hormone | Intramuscular | 222757 | DB13953 |
| benzoate | | | | |
| Estradiol | Hormone | Intramuscular | 9403 | DB13954 |
| cypionate | | | | |
| Estradiol | Hormone | Intramuscular | 165688 | DB13955 |
| dienanthate | | | | |
| Estradiol valerate | Hormone | Intramuscular | 13791 | DB13956 |
| Etodolac | Analgesic, antipyretic and anti- | Oral | 3308 | DB00749 |
| | inflammatory agent | | | |
| Etoposide | Antineoplastic agent | Oral, | 36462 | DB00773 |
| | | Intravenous | | |
| Ezetimibe | Anticholesteremic agent | Oral | 150311 | DB00973 |
| Ezogabine | Antiepileptic agent | Oral | 121892 | DB04953 |
| Flunitrazepam | CNS Depressant | Oral | 3380 | DB01544 |
| Flurbiprofen | Analgesic, antipyretic and anti- | Oral | 3394 | DB00712 |
| | inflammatory agent | | | |
| Fluvastatin | Anticholesteremic agent | Oral | 1548972 | DB01095 |
| Glecaprevir | Antiviral agent | Oral | 66828839 | DB13879 |
| Haloperidol | Neuroleptic agent | Oral, | 3559 | DB00502 |
| | | Intramuscular | | |
| Hydromorphone | Analgesic, antipyretic and anti- | Oral, | 5284570 | DB00327 |
| | inflammatory agent | Intramuscular, | | |
| | | Intravenous, | | |
| | | Subcutaneous | | |
| Ibuprofen | Analgesic, antipyretic and anti- | Oral, | 3672 | DB01050 |
| | inflammatory agent | Intravenous | | |
| Imidafenacin | Anticholinergic and | Oral | 6433090 | DB09262 |
| | parasympatholytic agent | | | |
| Indacaterol | Anti-asthmatic agent | Oral, | 6918554 | DB05039 |
| | | Inhalation | | |
| Indomethacin | Analgesic, antipyretic and anti- | Oral, Rectal, | 3715 | DB00328 |
| | inflammatory agent | Ophthalmic | | |
| Irbesartan | Antihypertensive agent | Oral | 3749 | DB01029 |
| Irinotecan | Antineoplastic agent | Intravenous | 60838 | DB00762 |
| Isavuconazole | Antifungal agent | Oral | 6918485 | DB11633 |
| Ketobemidone | Analgesic, antipyretic and anti- | Oral, | 10101 | DB06738 |
| | inflammatory agent | Intravenous | | |
| Lamotrigine | Antiepileptic agent | Oral | 3878 | DB00555 |
| Letermovir | Antiviral agent | Oral, | 45138674 | DB12070 |
| | | Intravenous | | |
| Licofelone | Analgesic, antipyretic and anti- | Oral | 133021 | DB04725 |
| | inflammatory agent | | | |
| Lorazepam | CNS Depressant | Oral, | 3958 | DB00186 |
| | | Sublingual, | | |
| | | Intramuscular, | | |
| | | Intravenous | | |
| Losartan | Antihypertensive agent | Oral | 3961 | DB00678 |
| Lovastatin | Anticholesteremic agent | Oral | 53232 | DB00227 |
| Lumiracoxib | Analgesic, antipyretic and anti- | Oral | 151166 | DB01283 |
| | inflammatory agent | | | |
| Midazolam | CNS Depressant | Buccal, | 4192 | DB00683 |
| | | Intramuscular; | | |
| | | Intravenous | | |
| Mitiglinide | Anti-diabetic agent | Oral | 121891 | DB01252 |
| Morphine; | Analgesic, antipyretic and anti- | Oral; Rectal, | 5288826 | DB00295 |
| Morphine Sulfate | inflammatory agent | Intramuscular, | | |
| | | Intravenous, | | |
| | | Subcutaneous, | | |
| | | Epidural | | |
| Muraglitazar | Anti-diabetic agent | Oral | 206044 | DB06510 |
| Mycophenolate | Immunosuppressant | Oral, | 5281078 | DB00688 |
| mofetil | | Intravenous | | |
| Mycophenolic | Immunosuppressant | Oral | 446541 | DB01024 |
| acid | | | | |
| Naldemedine | Analgesic, antipyretic and anti- | Oral | 54732242 | DB11691 |
| | inflammatory agent | | | |
| Nalmefene | Opioid antidote | Oral | 5284594 | DB06230 |
| Naltrexone | Opioid antidote | Oral, | 5360515 | DB00704 |
| | | Subcutaneous | | |
| Naproxen | Analgesic, antipyretic and anti- | Oral | 156391 | DB00788 |
| | inflammatory agent | | | |
| Nateglinide | Anti-diabetic agent | Oral | 5311309 | DB00731 |
| Oxazepam | CNS Depressant | Oral | 4616 | DB00842 |
| Paricalcitol | Anti-parathyroid agent | Oral, | 5281104 | DB00910 |
| | | Intravenous | | |
| Pibrentasvir | Antiviral agent | Oral | 58031952 | DB13878 |
| Pitavastatin | Anticholesteremic agent | Oral | 5282452 | DB08860 |
| Propofol | Anesthetic | Intravenous | 4943 | DB00818 |
| Raltegravir | Antiviral agent | Oral | 54671008 | DB06817 |
| Regorafenib | Antineoplastic agent | Oral | 11167602 | DB08896 |
| Rifampicin | Antibacterial agent | Oral, | 5381226 | DB01045 |
| | | Intravenous | | |
| Rucaparib | Antineoplastic agent | Oral | 9931954 | DB12332 |
| Seratrodast | Anti-asthmatic agent | Oral | 2449 | DB06739 |
| Silodosin | Anti-benign prostatic | Oral | 5312125 | DB06207 |
| | hyperplasia | | | |
| Simvastatin | Anticholesteremic agent | Oral | 54454 | DB00641 |
| Sorafenib | Antineoplastic agent | Oral | 216239 | DB00398 |
| Sulfamethoxazole | Antibacterial agent | Oral | 5329 | DB01015 |
| Suprofen | Analgesic, antipyretic and anti- | Oral, | 5359 | DB00870 |
| | inflammatory agent | Ophthalmic | | |
| Tamoxifen | Antineoplastic agent | Oral | 2733526 | DB00675 |
| Tapentadol | Analgesic, antipyretic and anti- | Oral | 9838022 | DB06204 |
| | inflammatory agent | | | |
| Testosterone | Hormone | Intramuscular | 5995 | DB01420 |
| propionate | | | | |
| Topiroxostat | Anti-gout | Oral | 5288320 | DB01685 |
| Trifluoperazine | Neuroleptic agent | Oral | 5566 | DB00831 |
| Troglitazone | Anti-diabetic agent | Oral | 5591 | DB00197 |
| Vadimezan | Antineoplastic agent | Oral | 123964 | DB06235 |
| Valdecoxib | Analgesic, antipyretic and anti- | Oral | 119607 | DB00580 |
| | inflammatory agent | | | |
| Valproic Acid | Antiepileptic agent | Oral, | 3121 | DB00313 |
| | | Intravenous | | |
| Zaltoprofen | Analgesic, antipyretic and anti- | Oral | 5720 | DB06737 |
| | inflammatory agent | | | |
| Zidovudine | Antiviral agent | Oral, | 35370 | DB00495 |
| | | Intravenous | | |
| Zileuton | Anti-asthmatic agent | Oral | 60490 | DB00744 |

### Experimental Procedures

### Construction of the GusR_K125A-Expressing Vector (DOIN Vector):

The pYF57 backbone (as denoted by SEQ ID NO: 1) is digested with the restriction enzymes XbaI and PsiI and purified from gel using commercial gel-extraction kit.

The GusR_K125A is synthesized and its sequence is verified (nucleic acid sequence is as denoted by SEQ ID NO: 2 and amino acid sequence is as denoted by SEQ ID NO: 3). The target sequenced is flanked with the XbaI and PsiI restriction sites.

The GusR_K125A fragment is digested with XbaI and PsiI and purified from gel.

The GusR_K125A is ligated into the pYF57 backbone using standard laboratory ligation procedures.

Positive clones are screened by colony-PCR and correct clones are verified by sequencing.

### Production of Phage Lysate:

Phages are designed to target enteric E. coli GUS-producing bacterial population.

The Escherichia coli K12 BW25113 strain (DSM 27469) is used as the host cell for the propagation of all types of phages.

The host cells harbor plasmid that encodes the tail genes (TC1 as denoted by SEQ ID NO: 4 or TC20 as denoted by SEQ ID NO: 5) to facilitate the propagation of intact active phages.

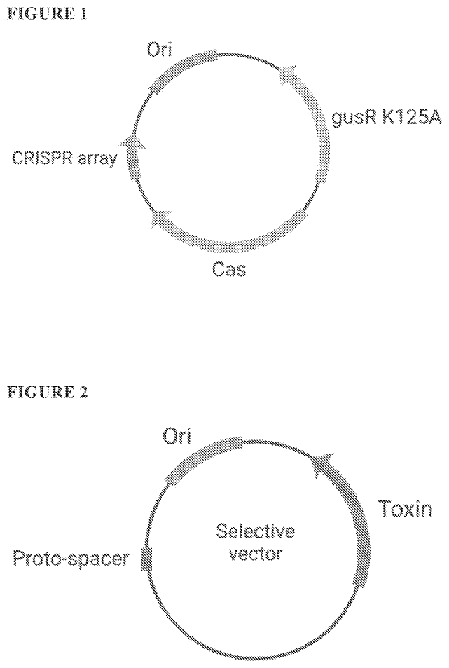

Two different types of host cell are used to produce two different types of phage-based particles:
i) For the production of phages containing the DOIN vector expressing GusR_K125A, the host cells also contain plasmid with packaging signal and the DOIN encoding sequence that is packed into the particles (FIG. 1). This plasmid is transduced into the targeted bacteria.
ii) For the production of phages containing the selective vector (nucleic acid sequence as denoted by SEQ ID NO: 6), the host cells also contain plasmid with packaging signal and bacterial toxin encoding sequence that are packed into the particles (FIG. 2). This plasmid is transduced into the targeted bacteria.

Over-night culture of the host cells grow in LB supplemented with the appropriate antibiotic/s at 37° C. and 220 RPM agitation.

On the next day, the cells are refreshed and the culture grow until reach OD₆₀₀ of 0.6.

The culture is then infected with T7 helper-phage (nucleic acid sequence as denoted by SEQ ID NO: 7) at a multiplicity of infection (MOI) of approximately 1. The used genome of the T7 helper-phage lacks the three essential structural genes encoding the phage tail module.

Three hours post infection chloroform is added to the culture followed by brief vortex.

The lysate titer is determined using the transducing forming units (TFU) assay as previously described elsewhere.

### Determination of the In-Vitro Effect of GusR K125A on GUS Activity:

Two different bacterial strains are examined: E. coli K-12 BW25113 and E. coli K12 MG1655 (ATCC 700926). These strains were shown to colonize human intestines and harbor the GUS operon.

The produced lysates are used to transduce each of the abovementioned bacterial hosts, and the transduction efficiencies for each strain is determined by the TFU assay.

The strain that demonstrates the highest transduction efficiencies is selected for the in-vitro GUS activity assay.

The GUS activity is performed as previously described elsewhere, including all required assay controls. Briefly, bacterial cultures grow to reach OD₆₀₀ of 0.6. Once reached the targeted growth phase, the culture is transduced with the GusR_K125A-expressing vector. One hour later, the bacterial cultures are added to the assay reaction, along with the substrate (PNPG) and the assay buffer. The assay reaction is incubated at 37° C. for 6 hours and the activity of the GUS operon is monitored by the presence of the hydrolysis product (PNP) by measuring the absorbance at 410 nm. The arbitrary units of the GUS operon specific activity are calculated as follows:

$$\frac{O . D 405}{O . D 600 \times Time ( min ) \times volume} \times 1000 =$$

In a series of follow-ups studies, the selective vector is used to demonstrate the enrichment in GUS-inhibited bacterial population over time.

### Demonstrate CTP-11 Alleviated Cytotoxicity of GUS-Inhibited Bacteria

Bacteria (WT and GUS-inhibitor producing bacteria) are grown and induced using PNPG as described above. Following induction, the bacteria are lysed, and extracts are incubated with SN-38G or PNPG. At the end of the incubation period, the PNPG-incubated bacterial extracts are taken to determine the activity of the GUS. In parallel, the SN-38G-incubated bacterial extracts are added to HT29 cell culture to determine cytotoxicity and CC50.

The CC50 of the different test items are determined using the MTT assay. Briefly, Cell suspensions containing 2×10⁴ viable cells/ml are seeded into 96-well microtiter plates. After a 48-h incubation, the medium is replaced with medium containing various compounds or bacterial extracts, and exposed for 48 hr. The plates are developed by adding 50 μl MTT at 2 mg/ml to each well and incubated for a further 3 h. The plates are centrifuged at 450 g for 10 min, and the supernatant is carefully aspirated. The formazan crystals are dissolved in 200 μl of DMSO, and the absorbance at 570 nm is measured, with background correction at 620 nm.

### Determination of the In-Vivo Effect of GusR K125A on Alleviating CTP-11 Toxicity:

An appropriate gut animal model is used to demonstrate in-vivo efficacy. For this aim, two preliminary steps are performed to establish the animal model: (a) chronic colonization establishment of the targeted bacterial cells and; (b), characterization of the bioavailability of the phage-based particles used for treatment. Once the animal model is established, efficacy studies are performed. The following two models are used in the following experimental steps: Sprague Dawley rats (250-290 gr) and −6-8 week old female Balb/cJ mice

In each animal model, the animals are divided into four experimental groups: group 1-vhicle control, group 2-treatment with phage-based particles containing the GusR_K125A-expressing vector and the with phage-based particles containing the selective vector at a pre-determined regimen, group 3-CPT-11 injected CPT-11 (50 mg/kg/day) and group 4-CPT-11 injected and treated with the above phage-based particles.
The study endpoints are the clinical parameters of body weight, diarrhea (fecal staining of skin, lose watery stool) and bloody diarrhea (black sticky stool). These parameters are monitored during the study (11 days).
At the end of the study, the large intestine samples are histopathologically examined, and histologic score for each sample is determined according to pre-defined parameters.

### Example 1

### Inhibition of the GUS Activity Using the GusR_K125A Mutant

As a proof of concept, the microbiome GUS enzymes were used, demonstrating the ability of the present disclosure to manipulate bacterial populations and thereby to modulate drug metabolism in a treated subjects harboring the modified bacterial populations. Modulation of the metabolism of CPT-11 is first examined. CPT-11 (Irinotecan) is one of the three commonly used chemotherapeutic agents for colon cancer. CPT-11 is a prodrug that is activated in the liver by carboxylesterases (CE) to SN-38, an antineoplastic topoisomerase I poison. Liver SN-38 is inactivated to SN-38G by UDP-glucuronosyltransferase (UGT) enzymes and sent to the intestines. Once in the intestines, SN-38G serves as a substrate for GUS enzymes that reactivate SN-38 in situ, and the active SN-38 in the intestinal lumen generates CPT-11 dose-limiting side-effects.

GUS is the product of the gusA gene, which in the GUS operon is followed by the inner-membrane GlcA-specific transporter gusB and nonspecific outer-membrane channel gusC genes. The GUS operons in the Enterobacteriaceae are under the control of the transcriptional repressor GusR. Only the Enterobacteriaceae family of human gut-associated Proteobacteria, including Escherichia, Salmonella, Klebsiella, Yersinia, and Shigella taxa maintain a GUS operon.

More specifically, the GUS operons are largely, but not universally, retained in human gut-associated Enterobacteriaceae since all sequenced strains of GusR-encoding Enterobacteriaceae taxa maintain a complete operon, with the exception of one Klebsiella and two Shigella strains, which harbor truncated GUS operon genes.

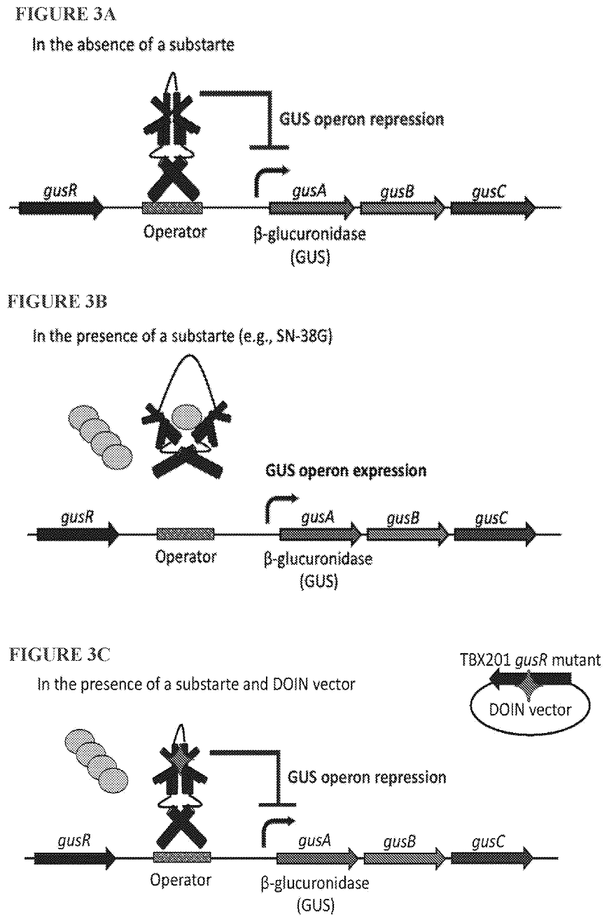

The crystal structure of the GusR has been determined and the domains important for the protein structure and function were characterized. It was found that GusR acts as a DNA-bound transcriptional repressor in the absence of ligand and releases from DNA once an appropriate glucuronide ligand is present. In vitro studies show that the release of GusR derepress the GUS operon, allowing for gusA expression and an increase of GUS enzyme (FIGS. 3A-3B).

Activity analysis of various GusR mutants reveal that replacement of lysine with alanine [K125A] (comprising the amino acid sequence as denoted by SEQ ID NO: 9, encoded by the nucleic acid sequence as denoted by SEQ ID NO: 8)] of the carboxylate-contacting residues produces GusR variants that have significantly lower affinity to the GUS ligand. Furthermore, it was shown that bacterial cells containing an expression plasmid for the K125A GusR variant exhibited no GUS activity even when induced with 1 mM PNPG (ligand-binding GUS). Since GUS was active in cells harboring empty vectors in the presence of ligand (due to endogenous GusR), the repression of GUS activity by the K125A variant is a dominant negative in cells that still retained their endogenous GusR protein (FIG. 3C).

A potent and specific GUS inhibitor, that reduces CPT-11-induced intestinal toxicity and would not hamper the treatment efficacy of CPT-11 nor disrupt the native microbiota, is of great clinical value. Moreover, the E. coli GUS shares 50% amino acid sequence identity with highly conserved active sites to human GUS, which is an important lysosomal enzyme for the degradation of glycosaminoglycan. Therefore, it is also important to specifically inhibit bacterial GUS but not the human GUS.

Using specific phage-based particles as the delivery vehicle for the two components of the system of the present disclosure, GUS-expressing gut bacterial cells with dominant negative GUS mutant variant suppressor (GUS_K125A) were performed. The expression of the dominant negative mutant that inactivates GUS gut activity, thus decrease microbial GUS-mediated SN-38 reactivation in the gut (FIG. 3C).

The following steps were therefore performed:
The transducing particles containing the GusR_K125A-expressing vector, as well as at least one CRISPR array (also referred to by the present disclosure as the modulating component) are delivered into the GI tract and there they specifically target GUS-producing host cells (e.g., E. coli cells).
The targeted host cells (e.g., E. coli) are transduced with the GusR_K125A-expressing vector, which result with the transcriptional repression of the GUS operon.
Following treatment with particles containing the GusR_K125A-expressing vector, the selective pressure to maintain repression of the GUS operon by the targeted cells is achieved by the treatment with the selective component (e.g., phage-based particles that encode a toxic element, but also comprise at least one protospacer recognized by the CRISPR array of the modulating component.
Only host cells that hold the GusR_K125A enzyme and the CRISPR array survive the lethal attack of the selective particles.
Repeated enrichment cycles using the selective pressure, favoring the GUS-repressed bacterial population, results with suppression of GUS production in the gut, alleviating SN-38 GUS-mediated toxicity.

### Inhibition of the GUS Activity Using the GusR_K125A Mutant

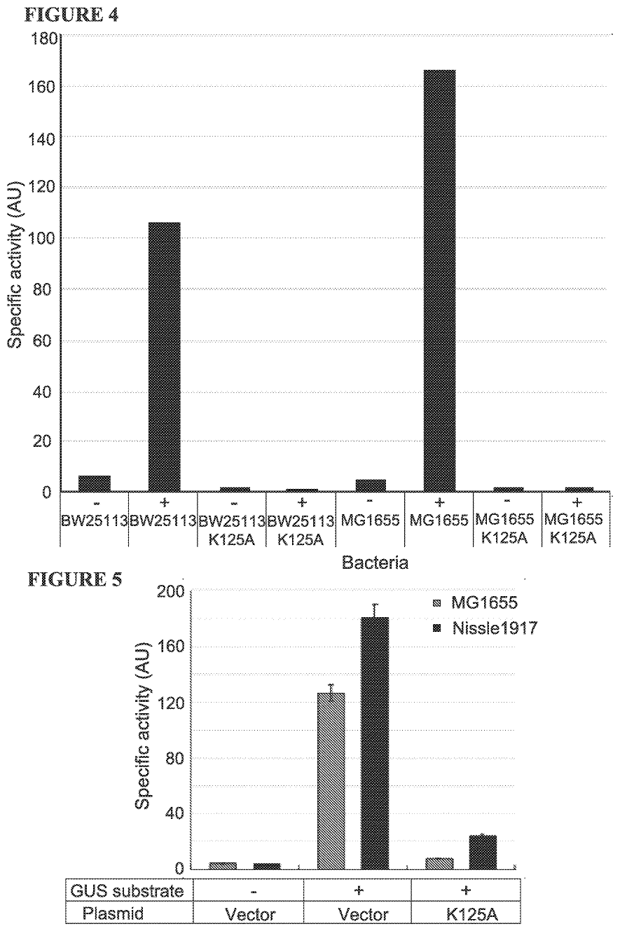

Inhibition of the GUS activity using the GusR_K125A mutant was next evaluated by the inventors. More specifically, two E. coli bacterial hosts were used: BW25113 and MG1655. A plasmid that expresses the mutant repressor K125A was introduced into each of these hosts (BW25113 K125A and MG1655 K125A). The activity of the GUS operon was determined in the absence (−) or presence (+) of the GUS operon inducer PNPG. The specific units were calculated as described above. As shown by FIG. 4, GUS activity is inhibited in the presence of the dominant negative mutant repressor K125A in two bacterial hosts.

The inventors next evaluated the ability of the GusR mutant to inhibit GUS enzymatic activity in an additional microbiome isolate. Therefore, the E. coli MG1655 and Nissle1917 strains were next examined. These strains were chosen for testing because relevant to the intendent use of the disclosed system both strains were found to be present in the human microbiome [Wallace B D, W. H. (2010), Science, November 5; 330 (6005): 831-5; Turnbaugh P J, L. R.-L. (2007) Nature., October 18; 449 (7164): 804-10]. Moreover, the Nissle1917 is considered the microbiome model organism, and it is clinically investigated as a biological therapeutic [U., S. (2016) FEMS Microbiol Lett. October; 363 (19): fnw212].

FIG. 5 shows that the K125A GusR mutant significantly inhibits the GUS activity in both bacterial strains also (8 to18-fold reduction).

These results clearly demonstrate effective and successful targeting of cells and establish the feasibility of modifying the metabolism of GUS substrate. More importantly, these results confirm that the K125A GusR mutant is applicable in various host, which are commensal bacteria in human microbiome, thereby establishing the potential therapeutic application of the disclosed system.

As indicated above, the inventors first established successful delivery of the GusR mutants to target cells and demonstrated the effective inhibition of GUS activity in bacterial cells harboring the GusR_K125A mutant. More specifically, phage-based particle lysate was prepared as described above. The transduction efficiency of the particles against the indicated bacterial host above was determined using the TFU assay, as described previously, and presented in Table 2. The tail used for transduction is TC1 (TC1 as denoted by SEQ ID NO: 4).

TABLE 2
Transduction efficacies of plasmid into bacterial hosts

| Host | TFU/ml |
|---|---|
| BW25113 | 2.3*10⁷ |
| MG1655 | 1.1*10⁷ |

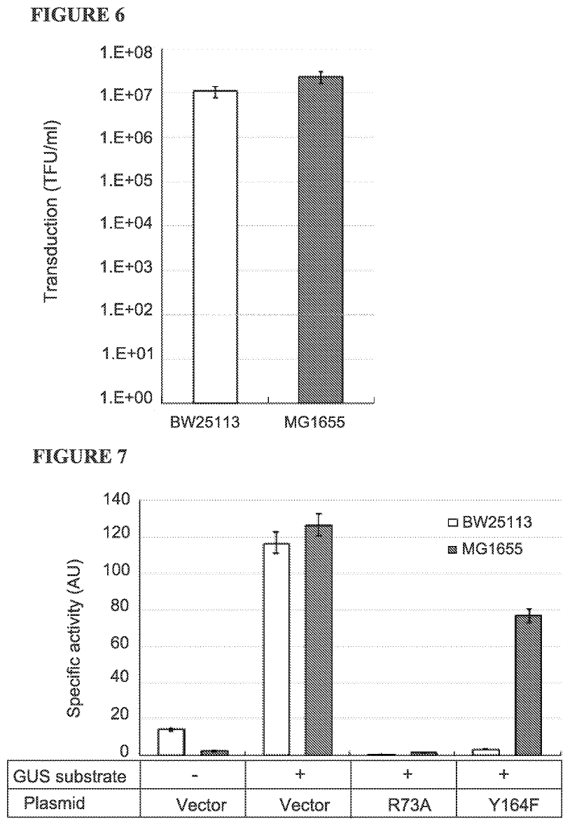

FIG. 6 demonstrates another successful transduction of the modulatory GusR mutant-CRISPR payload into microbiome isolates. The transduction efficiencies of the TBX201 payload into different microbiome host strains was determined using the TFU assay, as described above.

### Example 2

### The Bacterial GUS Enzyme is Inhibited by Two Additional GusR Mutated Variants

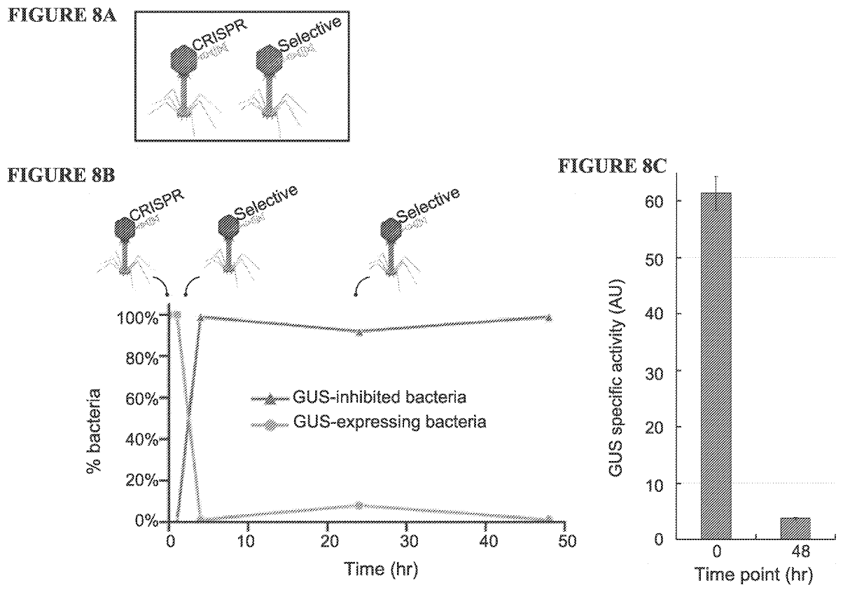

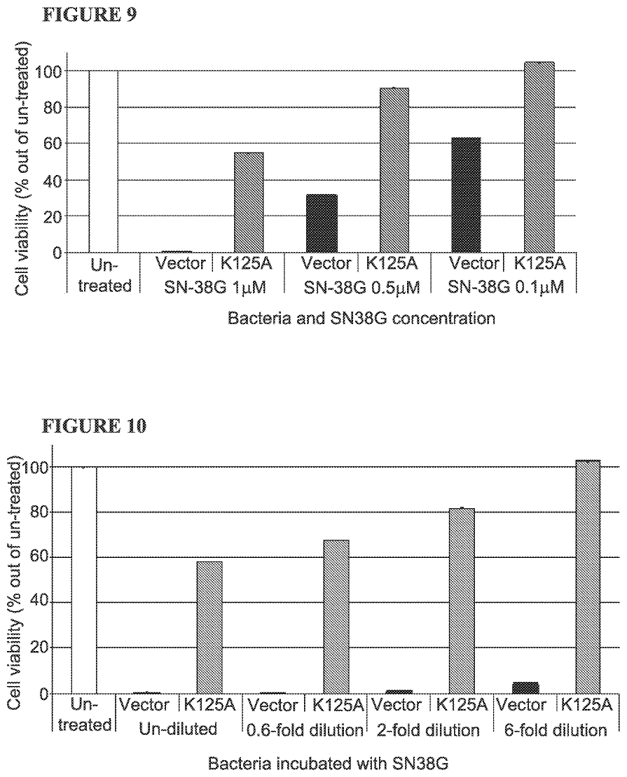

To evaluate the inhibition capacity of different variants of the GusR, two additional variants of the E. coli GusR repressor protein, the R73A (comprising the amino acid sequence as denoted by SEQ ID NO: 13, encoded by the nucleic acid sequence as denoted by SEQ ID NO: 12) and the Y164F (comprising the amino acid sequence as denoted by SEQ ID NO: 11, encoded by the nucleic acid sequence as denoted by SEQ ID NO: 10) mutants, were next constructed. These two mutants display different affinities to the GUS operon regulatory region. More specifically, since these two mutant variants are expected to have higher affinities to the GUS substrate, a lower inhibition capacity is expected. Both additional mutants were examined as means to improve the fitness of the transduced bacteria, as they display a more moderate effect on the sugar bacterial metabolism. FIG. 7 shows, that while the R73A mutant significantly inhibit the GUS enzymes in both tested bacteria (8 to 386 fold reduction), the inhibition capacity of the Y164F mutant is strain dependent. Specifically, in BW25113 bacteria the GUS activity was reduced by 34 fold but in MG1655 bacteria the activity was merely reduced by ˜1.5 fold. These findings indicate that the reduced inhibition of the Y164F mutant in the MG1655 bacteria, may result from the stronger substrate affinity of the Y164F mutant, as compared to the K125A mutant.

### Example 3

### Successful Conversion of Bacterial Population In-Vitro PoC

To demonstrate sustained conversion of above 99% of the GUS-inhibited bacterial population in-vitro, the system of the present disclosure has been utilized to inhibit the GUS in target bacteria and to enrich the bacterial culture with the desired GUS-inhibited population.

More specifically, the platform (also referred to herein as a system) of the present disclosure is composed of two components provided in two transducing particles (e.g., phages), the modulating components (also referred to herein as the “GusR mutant-CRISPR”, or “CRISPR”), and the selective component (also referred to herein as “Selective”), which are used sequentially (FIG. 8A). In a first step, the “CRISPR” phages transduce the GusR mutant payload, which inhibits bacterial GUS and express the CRISPR system. Thereafter, the “Selective” component phages are used to kill the remaining bacteria that were not transduced with the GusR mutant payload. The CRISPR system in the modulatory component together with the GusR mutant payload, renders the successfully transduced bacteria protected against the “Selective” phages. The system results with bacterial population enriched with GUS-inhibited bacteria.

The PoC of this system is demonstrated in FIGS. 8B, 8C. Mid-exponential BW25113 bacterial culture, was incubated with the modulating component encoding the GusR mutant and the CRISPR array (also referred to herein as “CRISPR” phages) at timepoint 0 hr. Two hours later, at time point 2 hr, approximately 0.1% of the bacteria in the culture contained the GusR mutant-CRISPR payload and became GUS-inhibited. At timepoint 2 hr and 24 hr, the “Selective” phages were used to selectively kill the remaining GUS-expressing bacteria that were not transduced by the “GusR mutant-CRISPR phages”. The selective killing by the “Selective” phages enriched the desired bacterial population and resulted in a sustained GUS-inhibited bacterial culture. As shown in FIG. 8B, at the end of the study (time point 48 hr) the majority (>99%) of the bacterial population was composed of GUS-inhibited bacteria.

It should be noted that in spite the fact that only 0.1% of the bacterial population initially transduced by the modulating component of the invention, the “CRISPR” phages, the disclosed platform successfully selected and enriched the desired GUS-inhibited bacterial population. Transduction of the “CRISPR” phages to the majority of the of the bacterial population, and the repeated use of the “Selective” phages, thus maximizes efficacy.

To determine how the conversion of the bacterial population affected the bacterial GUS enzyme activity, the culture was tested before and after the conversion. As shown in FIG. 8C, a significant reduction (16-fold) in the GUS activity was observed in the bacterial culture after conversion, compared to the bacterial culture prior to conversion.

These results demonstrate effective conversion of bacterial population and the successful manipulation of the enzymatic activity displayed by enzymes of the transduced bacterial strains, thereby modulating the composition and function of microbiome populations.

### Example 4

### GUS Inhibition in Bacteria Successfully Reduce Irinotecan Cytotoxicity

To demonstrate that inhibiting the bacterial GUS in the target bacterial hosts prevents the conversion of the inactivated SN38G into the active SN38 form, the GUS assay was performed as described above, using SN38G as the reaction substrate, instead of the experimental substrate PNPG. The rate of SN38G conversion to SN38 by the bacterial GUS in the samples is dependent on the reaction conditions (e.g., GUS enzyme activity, substrate concentration etc.), thus the resulting samples contained different concentrations of SN38. The samples were then incubated with HL29, human colorectal adenocarcinoma cell-line, and the viability of the cells was determined. Different concentrations of SN38G and bacteria were examined to evaluate their effect on the cytotoxicity of SN38. FIG. 9 demonstrates the effect of bacterial GUS inhibition on cells viability, using different SN38G concentrations. When the highest SN38G concentration was used (1 μM), the cells that were incubated with vector-expressing bacteria died, while in the same concentration, 60% of the cells incubated with K125A-expressing bacteria rescued. As the concentration of SN38G decreased, the viability of the cells increased, although in all cases, cells viability was higher when incubated with samples from K125A-expressing bacteria, compared to cells incubated with samples from empty vector-expressing bacteria. In the lowest SN38G concentration (0.1 μM), H29T cells incubated with samples obtained from the K125A GusR expressing bacteria. Incubation of the cells with these samples reveled that they were completely rescued from the toxic effect of SN38.

This study demonstrated that the inhibition of the bacterial GUS by the modulatory component, specifically, the GusR mutant K125A, hindered the conversion of the inactivated Irinotecan metabolite SN-38G, into the active SN-38 metabolite that display toxic effect on cells, thereby, reducing the cytotoxic effect of SN-38 on human cells. Moreover, these results show that the ability to rescue cells from SN-38 cytotoxicity depend on the initial concentration of SN-38G in the bacterial reactions, the more SN-38G is used as a substrate for the bacterial GUS, the more it is converted to SN-38 by the enzyme, therefore the more toxic the sample is to the cells.

The effect of the bacterial concentration, and consequently the GUS enzyme concentration, on the conversion rate of SN-38G and cell viability, was also examined. In this assay, before adding SN-38G (1 μM), the bacteria were diluted to test different GUS concentrations.

As shown in FIG. 10, when the highest bacterial concentration was used (Un-diluted), the same result was obtained when tested in the same conditions as presented in FIG. 9, (SN38G 1 μM). The more diluted the bacterial concentration (i.e., the more diluted GUS), the viability of cells incubated with K125A-expressing bacterial samples increased, while the cells incubated with vector-expressing bacterial samples did not survive. The maximal effect of rescuing remarkably nearly 100% of the cells from the SN38 toxicity was reached when the bacteria were 6-fold diluted.

In mice, it was found that the peak concentration of SN38 was ˜1 μg/gr tissue weight, reached 2 hours after administration of Irinotecan at the dose of 80 mg/kg (Li K, Wang S. s.l.: AAPS PharmSciTech., 2016, Vols. December; 17 (6): 1450-1456). Taking into consideration that the large intestinal of mice weights around 1 gr (Ogiolda L, Wanke R, Rottmann O, Hermanns W, Wolf E. s.l.: Anat Rec., 1998, Vols. March; 250 (3): 292-9), the total amount of intestinal SN38 is 1 μg, approximately twice the amount of its substrate SN38G that was used here. Moreover, as the concentration of target bacteria in the intestine (˜108 bacteria per gr gut content) is similar to that used in the above experiments (108 bacteria per ml). (Macfarlane G T, Macfarlane S, Gibson G R. s.l.: Microb Ecol., 1998, Vols. March 35 (2): 180-7), it is reasonable to assume that the experiment conditions tested herein in-vitro, fairly resemble to those occurring in the intestines of animals in-vivo.

To confirm that the alleviated cytotoxicity of the K125A samples indeed resulted from the inhibition of GUS in these bacteria, the GUS assay was performed as above, testing the activity of the enzyme in these samples. Indeed, the GUS specific activity in the K125A-expressing bacteria were around 10-fold lower than in the empty vector-expressing bacteria.

Taken together, as demonstrated herein, inhibition of the bacterial GUS by the modulating component of the present disclosure, reduced Irinotecan cytotoxicity in human cell-line by preventing the conversion of SN38G to SN38 (Irinotecan), the toxic metabolite of Irinotecan. Moreover, the alleviation of Irinotecan toxicity was shown to be dose-depended by the capacity of the bacterial GUS inhibition.

### Example 5

### Efficacy of the Disclosed System—In Vivo Animal Model

A well-known animal model to evaluate the effect of the system of the present disclosure on Irinotecan (CPT-11) toxicity is used [Wang H, et 1., Science, 2010, Vols. November 5; 330 (6005): 831-5; Bhatt A P, et al.]. Briefly, Sprague Dawley rats (250-290 gr) are randomly allocated to the following study groups (n=8): group 1, no CPT-11 and no modulatory system of the present disclosure; group 2, CPT-11 and no modulatory system of the present disclosure; group 3, no CPT-11 and with the modulatory system of the present disclosure; group 4, with CPT-11 and with the modulatory system of the present disclosure. Rats of all groups are allowed to acclimate.

To enable direct administration of the phages, a silastic tube (cannula) is inserted to the animal's jejunum via enterotomy. Animals are allowed to recover from surgery and then vehicle or Trobix Bio phages are directly administered into the GI tract through the jejunum cannula, once a day for three consecutive days. CPT-11 (50 mg/kg/day) or saline then are injected intraperitoneally for 10 consecutive days.

The rats are monitored daily for signs of diarrhea, body weight and food consumption. A grading index for diarrhea severity ranging from 0 to 3 is used:
0—indicating solid stool (i.e., normal stool)
1—indicating slightly wet and soft stool (slight diarrhea)
2—indicating wet and unformed stool with moderate perianal staining of the coat (moderate diarrhea)
3—indicating watery stool with severe perianal staining of the coat (severe diarrhea)

At the study end, after sacrifice, jejunum, ileum, and colon samples are harvested and fixed. The prepared slides are stained for histopathological evaluation.

### Example 6

### GUS-Metabolized Drugs, In Vitro and In Vivo Experiments

Examples of medications that are metabolized by the human UGT enzymes and can be potentially affected by the microbiome GUS enzymes, along with their therapeutic categories, are detailed in Table 1 above.

The in vitro cell cytotoxicity assays and the in vivo animal studies as described for Irinotecan in Examples 4 and 5, are performed for each of the following GUS substrate drugs: Abacavir, Acemetacin, Acetaminophen, Ambrisentan, Artenimol, Asenapine, Atorvastatin, Axitinib, Bictegravir, Buprenorphine, Canagliflozin, Candesartan, Candesartan cilexetil, Carbamazepine, Chenodeoxycholic acid, Clozapine, Codeine, Cyproheptadine, Dabigatran etexilate, Dapagliflozin, Deferiprone, Delafloxacin, Diclofenac, Diflunisal, Dolutegravir, Eltrombopag, Enasidenib, Entacapone, Epirubicin, Erlotinib, Ertugliflozin, Estradiol, Estradiol acetate, Estradiol benzoate, Estradiol cypionate, Estradiol dienanthate, Estradiol valerate, Etodolac, Etoposide, Ezetimibe, Ezogabine, Flunitrazepam, Flurbiprofen, Fluvastatin, Glecaprevir, Haloperidol, Hydromorphone, Ibuprofen, Imidafenacin, Indacaterol, Indomethacin, Irbesartan, Irinotecan, Isavuconazole, Ketobemidone, Lamotrigine, Letermovir, Licofelone, Lorazepam, Losartan, Lovastatin, Lumiracoxib, Midazolam, Mitiglinide, Morphine; Morphine Sulfate, Muraglitazar, Mycophenolate mofetil, Mycophenolic acid, Naldemedine, Nalmefene, Naltrexone, Naproxen, Nateglinide, Oxazepam, Paricalcitol, Pibrentasvir, Pitavastatin, Propofol, Raltegravir, Regorafenib, Rifampicin, Rucaparib, Seratrodast, Silodosin, Simvastatin, Sorafenib, Sulfamethoxazole, Suprofen, Tamoxifen, Tapentadol, Testosterone propionate, Topiroxostat, Trifluoperazine, Troglitazone, Vadimezan, Valdecoxib, Valproic Acid, Zaltoprofen, Zidovudine or Zileuton, as also disclosed in Table 1.

The following Table 3 lists all sequences disclosed by the present disclosure in the attached sequence listing:

TABLE 3
sequences of the present disclosure

| SEQ ID NO. | DESCRIPTION |
|---|---|
| 1 | AFTIFICIAL |
| | pYF57 |
| 2 | GusR-WT, DNA |
| 3 | GusR-WT, PROTEIN |
| 4 | Tail plasmid-TC1 |
| 5 | Tail plasmid-TC20 |
| 6 | Selective vector |
| 7 | T7 helper-phage |
| 8 | GusR_K125A (uidR AYG19327.1) |
| 9 | GusR_K125A (uidR AYG19327.1) |
| 10 | GusR-Y164F, DNA |
| 11 | GusR-Y164F, PROTEIN |
| 12 | GusR-R73A, DNA |
| 13 | GusR-R73A, PROTEIN |
| 14 | GusA, DNA |
| 15 | GusA, PROTEIN |
| 16 | GusB, DNA |
| 17 | GusB, PROTEIN |
| 18 | GusC, DNA |
| 19 | GusC, PROTEIN |
| 20 | Non-coding regulatory region (GusR |
| | operator) |
| | Located between GusR and GusA |

1-60. (canceled)

61. A system for modulating drug metabolism by modifying target cell/s and/or population of cells comprising said target cell/s, comprising:
(a) at least one drug metabolism modulating component comprising at least one nucleic acid sequence encoding and/or modulating at least one element participating in metabolism of at least one drug, at least one cas gene and at least one Clustered, Regularly Interspaced Short Palindromic Repeat (CRISPR) array; and
(b) at least one selective component comprising at least one protospacer, wherein said at least one protospacer is targeted by at least one spacer of the CRISPR array of (a), thereby inactivating said selective component.

62. The system of claim 61, wherein at least one of:
(a) said modulating component is comprised within at least one delivery vehicle that specifically targets said cell/s and/or said cells in a cell population; optionally, said at least one delivery vehicle is, or comprises at least one genetic element, said genetic element is at least one of: at least one transducing particle, at least one bacteriophage or any fragments or parts thereof, at least one bacteriophage-based or bacteriophage-like transducing particle and/or at least one modified bacteriophage, at least one vector, plasmid and/or construct, that comprises and/or encodes at least one of said modulating component and any combinations or cocktails thereof;
(b) said selective component is comprised within at least one delivery vehicle that specifically targets said cell/s and/or said cells in a cell population, said selective component comprises at least one agent that affects cell viability and/or activity; optionally, said at least one delivery vehicle is, or comprises at least one genetic element, said genetic element is at least one of: at least one transducing particle, at least one bacteriophage or any fragments or parts thereof, at least one bacteriophage-based or bacteriophage-like transducing particle and/or at least one modified bacteriophage, at least one vector, plasmid and/or construct, that comprises and/or encodes at least one of said selective component and any combinations or cocktails thereof, wherein said delivery vehicle is at least one bacteriophage or any bacteriophage-like, or bacteriophage-based transducing particle, that comprises and/or encodes at least one toxic element that kill bacterial cells or disrupt, attenuate, and/or inhibit bacterial growth and/or function;
(c) said target cell is a bacterial cell, and wherein said bacterial cell/s and/or cell population is or comprised within gut microbiome cell population, said cell population comprises at least one bacteria of the Proteobacteria, Firmicutes, Bacteroidetes, Actinobacteria, and phyla, or any mutant, variant of isolate or any combination thereof; and
(d) said drug metabolism modulating component affects at least one of the activity, bioavailability, clearance, stability, toxicity, and absorption of said drug.

63. The system of claim 61, wherein said element that participates in the metabolism of said at least one drug is at least one glucuronide enzyme, optionally, wherein at least one of:
(a) said at least one glucuronide enzyme is at least one β-glucuronidase (GUS) enzyme;
(b) said modulating component comprises nucleic acid sequence that encodes at least one element that inhibits and/or reduces the expression and/or activity and/or stability of at least one βGUS enzyme;
(c) said element encoded by the nucleic acid sequence of said modulating component represses the transcription of said βGUS enzyme, optionally, wherein said element is a Gus Repressor (GusR);
(d) said GusR display reduced or abolished affinity to at least one GUS ligand and/or substrate; and
(e) said GusR carry at least one mutation in at least one of: lysine (K) 125 to Alanine (A), Tyrosine (Y) 164 to Phenylalanine (F), and Arginine (R) 73 to Alanine (A), and any combinations thereof.

64. The system of claim 61, wherein said at least one drug is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, cns depressant, anticholinergic agent, parasympatholytic agent, immunosuppressant, opioid antidote, anti-asthmatic agent, anti-benign prostatic hyperplasia or anti-gout agent, optionally, wherein at least one of:
(a) said drug is at least one antineoplastic agent, optionally, at least one topoisomerase I inhibitor; and
(b) said drug is at least one topoisomerase I inhibitor and wherein said topoisomerase I inhibitor is Camptothecin-11 (CPT-11, Irinotecan), or any derivatives, metabolites or biosimilars thereof.

65. The system of claim 61, wherein said drug metabolism modulating component inhibits or reduces the re-activation and/or conversion of the non-active metabolite of said CPT-11, SN38glucuronide (SN38G) to the active metabolite of said CPT-11, SN38 by bacterial GUS.

66. A method for modulating drug metabolism of a target cell/s and/or population of cells comprising said target cell/s, the method comprising at least one of:
(a) contacting said cell/s or any cell population comprising said cell/s with at least one drug metabolism modulating component comprising at least one nucleic acid sequence encoding or modulating at least one element participating in metabolism of at least one drug, at least one cas gene and at least one CRISPR array; and
(b) contacting said cells or any cell population comprising said cell/s with at least one selective component comprising at least one protospacer, wherein said at least one protospacer is targeted by at least one spacer of the CRISPR array of (a) to inactivate said selective component;
thereby modulating drug metabolism of said bacterial cell and/or obtaining a population of bacterial cells exhibiting modified metabolism of at least one drug.

67. The method of claim 66, wherein at least one of:
(a) said modulating component is comprised within at least one delivery vehicle that specifically targets said cell/s and/or said cells in a cell population; optionally, said at least one delivery vehicle is, or comprises at least one genetic element, said genetic element is at least one of: at least one transducing particle, at least one bacteriophage or any fragments or parts thereof, at least one bacteriophage-based or bacteriophage-like transducing particle and/or at least one modified bacteriophage, at least one vector, plasmid and/or construct, that comprises and/or encodes at least one of said modulating component and any combinations or cocktails thereof;
(b) said selective component is comprised within at least one delivery vehicle that specifically targets said cell/s and/or said cells in a bacterial cell population, said selective component comprises at least one agent that affects cell viability and/or activity; optionally, said at least one delivery vehicle is, or comprises at least one genetic element, said genetic element is at least one of: at least one transducing particle, at least one bacteriophage or any fragments or parts thereof, at least one bacteriophage-based or bacteriophage-like transducing particle and/or at least one modified bacteriophage, at least one vector, plasmid and/or construct, that comprises and/or encodes at least one of said selective component and any combinations or cocktails thereof, said delivery vehicle is at least one bacteriophage or any bacteriophage-like or bacteriophage-based transducing particle that comprises and/or encodes at least one toxic element that kill bacterial cells or disrupt, attenuate, and/or inhibit bacterial growth and/or function;
(c) said target cell is a bacterial cell, and wherein said bacterial cell/s and/or cell population is or comprised within gut microbiome cell population, said cell population comprises at least one bacteria of the Firmicutes, Bacteroidetes, Actinobacteria, and Proteobacteria phyla, or any mutant, variant of isolate or any combination thereof; and
(d) said drug metabolism modulating component affects at least one of the activity, bioavailability, clearance, stability, toxicity, and absorption of said drug.

68. The method of claim 66, wherein said element that participates in the metabolism of said at least one drug is at least one glucuronide enzyme, optionally, at least one of:
(a) said at least one glucuronide enzyme is at least one β-glucuronidase (GUS) enzyme;
(b) said modulating component comprises nucleic acid sequence that encodes at least one element that inhibits and/or reduces the expression and/or activity and/or stability of at least one β-glucuronidase (GUS) enzyme;
(c) said element encoded by the nucleic acid sequence of said modulating component represses the transcription of said βGUS enzyme, optionally, wherein said element is a Gus Repressor (GusR); and
(d) said GusR display reduced or abolished affinity to at least one GUS ligand and/or substrate.

69. The method of claim 68, wherein said GusR carry at least one mutation in at least one of:
lysine (K) 125 to Alanine (A), Tyrosine (Y) 164 to Phenylalanine (F), and Arginine (R) 73 to Alanine (A), and any combinations thereof.

70. The method of claim 66, wherein said at least one drug is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, cns depressant, anticholinergic agent, parasympatholytic agent, immunosuppressant, opioid antidote, anti-asthmatic agent, anti-benign prostatic hyperplasia or anti-gout agent; optionally, at least one of:
(a) said drug is at least one antineoplastic agent, optionally, at least one topoisomerase I inhibitor;
(b) said drug is at least one topoisomerase I inhibitor, and wherein said topoisomerase I inhibitor is Camptothecin-11 (CPT-11, Irinotecan), or any derivatives, metabolites or biosimilars thereof; and
(c) said drug metabolism modulating component inhibits or reduces the re-activation and/or conversion of the non-active metabolite of said CPT-11 by bacterial GUS.

71. The method of claim 66, wherein step (b) is repeated at least twice to enrich said population of modified cells displaying modulated metabolism of at least one drug.

72. At least one cell and/or a population of said cells, or any composition or product thereof, exhibiting modified metabolism of at least one drug, wherein said cell and/or cell population is prepared by a method comprising at least one of:
(a) contacting said cell/s or any cell population comprising said cell/s with at least one drug metabolism modulating component comprising at least one nucleic acid sequence encoding or modulating at least one element participating in metabolism of at least one drug, at least one cas gene and at least one CRISPR array; and
(b) contacting said cells or any cell population comprising said cell/s with at least one selective component comprising at least one protospacer, wherein said at least one protospacer is targeted by at least one spacer of the CRISPR array of (a), to inactivate said selective component.

73. The cell and/or a population comprising said cell of claim 72, wherein at least one of:
(a) said modulating component comprises nucleic acid sequence that encodes at least one element that inhibits and/or reduces the expression and/or activity and/or stability of at least one β-glucuronidase (GUS) enzyme; optionally, at least one of:
(i) wherein the element encoded by the nucleic acid sequence of said modulating component represses the transcription of said βGUS enzyme, optionally, wherein said element is a GusR; and
(ii) wherein said GusR display reduced or abolished affinity to at least one GUS ligand and/or substrate, optionally, wherein said GusR carry at least one mutation in at least one of:
lysine (K) 125 to Alanine (A), Tyrosine (Y) 164 to Phenylalanine (F), and Arginine (R) 73 to Alanine (A), and any combinations thereof;
(b) said drug is at least one antineoplastic agent, optionally, at least one topoisomerase I inhibitor, optionally, at least one of:
(i) said drug is at least one topoisomerase I inhibitor, and wherein said topoisomerase I inhibitor is Camptothecin-11 (CPT-11, Irinotecan), or any derivatives, metabolite/s and biosimilar/s thereof; and
(ii) said cell display inhibited or reduced re-activation and/or conversion of the non-active metabolite of said CPT-11, SN38G, to the active metabolite of said CPT-11, SN38 by GUS.

74. A pharmaceutical composition or kit comprising:
(I) at least one drug; and at least one of:
(II) at least one drug metabolism modulatory system according to claim 61, said system comprising at least one of:
(a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and
(b) at least one selective component comprising at least one protospacer, wherein said at least one protospacer is targeted by at least one spacer of the CRISPR array of (a), to inactivate said selective component; and
(III) at least one bacterial cell and/or a population of said bacterial cells, or any composition or product thereof, exhibiting modified metabolism of said at least one drug, wherein said cells are prepared by contacting the cells with the system as defined by claim 61;
said composition optionally further comprises at least one of pharmaceutically acceptable carrier/s, diluent/s, excipient/s and additive/s, optionally, wherein at least one of:
(i) said at least one drug is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, CNS depressant, anticholinergic agent, parasympatholytic agent, immunosuppressant, opioid antidote, anti-asthmatic agent, anti-benign prostatic hyperplasia or anti-gout agent;
(ii) said drug is at least one antineoplastic agent, optionally, at least one topoisomerase I inhibitor; and
(iii) said drug is Camptothecin-11 (CPT-11, Irinotecan), or any derivatives, metabolite and biosimilar/s thereof; and

75. A method for modulating the metabolism of at least one drug in a subject in need thereof, the method comprising the step of administering to said subject an effective amount of at least one of:
(I) at least one drug metabolism modulatory system according to claim 61, said system comprising at least one of:
(a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and
(b) at least one selective component comprising at least one protospacer, wherein said at least one protospacer is targeted by at least one spacer of the CRISPR array of (a), to inactivate said selective component;
(II) at least one cell and/or a population of said cells, or any composition or product thereof, exhibiting modified metabolism of said at least one drug;
(III) at least one composition, kit or system comprising at least one of (I) and (II); and
(IV) any combination of said at least one of (I), (II) and (III) with said at least one drug.

76. A method for treating, preventing, ameliorating, reducing or delaying the onset of at least one pathologic disorder in a subject in need thereof, the method comprising the step of administering to said subject an effective amount of at least one drug and at least one of:
(I) at least one drug metabolism modulatory system according to claim 61, said system comprising at least one of:
(a) at least one drug metabolism modulating component comprising at least one nucleic acid sequence encoding or modulating at least one element participating in metabolism of at least one drug, at least one cas gene and at least one Clustered, Regularly Interspaced Short Palindromic Repeat (CRISPR) array; and
(b) at least one selective component comprising at least one protospacer, wherein said at least one protospacer is targeted by at least one spacer of the CRISPR array of (a), to inactivate said selective component;
(II) at least one bacterial cell and/or a population of said bacterial cells, or any composition or product thereof, exhibiting modified metabolism of said at least one drug;
(III) at least one composition, kit or system comprising at least one of (I) and (II); and
(IV) any combination of said at least one of (I), (II) and (III) with said at least one drug.

77. The method according to claim 76, wherein said pathologic disorder is at least one of a neoplastic disorder, a viral infection, an inflammatory disorder, an immune-cell mediated disorder, a metabolic disorder and an autoimmune disorder, optionally, wherein said subject is suffering of at least one neoplastic disorder.

78. The method according to claim 76, wherein said at least one drug is any one of an antineoplastic agent, antiviral agent, analgesic, antipyretic, anti-inflammatory agent, analgesic, antihypertensive agent, antimalarial agent, neuroleptic agent, anticholesteremic agent, anti-diabetic agent, antiepileptic agent, bile acid, anticoagulant, iron chelator, antibacterial agent, hematopoietic growth factor, antiparkinsonism agent, hormone, cns depressant, anticholinergic agent, parasympatholytic agent, immunosuppressant, opioid antidote, anti-asthmatic agent, anti-benign prostatic hyperplasia or anti-gout agent; optionally, at least one of:
(a) said drug is at least one antineoplastic agent, optionally, at least one topoisomerase I inhibitor; and
(b) said drug is at least one topoisomerase I inhibitor, and wherein said topoisomerase I inhibitor is Camptothecin-11 (CPT-11, Irinotecan), or any derivatives thereof.

79. The method according to claim 76, wherein said element encoded by the nucleic acid sequence of said modulating component represses the transcription of said βGUS enzyme, optionally, at least one of:
(a) said element is a Gus Repressor (GusR); and
(b) said GusR display reduced or abolished affinity to at least one GUS ligand and/or substrate.

80. The method of claim 79, wherein said GusR carry at least one mutation in at least one of: lysine (K) 125 to Alanine (A), Tyrosine (Y) 164 to Phenylalanine (F), and Arginine (R) 73 to Alanine (A), and any combinations thereof.
